SEQUENCE LISTING (45 sequences)

SEQ ID NO: 1 - protein, 332 aa, Shigella flexneri
MNITTLTNSISTSSFSPNNTNGSSTETVNSDIKTTTSSHPVSSLTMLNDTLHNIRTTNQALKKELSQKTLTKTSLEEIALHSSQISMDVNKSAQLLDILSRNEYPINKDARELLHSAPKEAELDGDQMISHRELWAKIANSINDINEQYLKVYEHAVSSYTQMYQDFSAVLSSLAGWISPGGNDGNSVKLQVNSLKKALEELKEKYKDKPLYPANNTVSQEQANKWLTELGGTIGKVSQKNGGYVVSINMTPIDNMLKSLDNLGGNGEVVLDNAKYQAWNAGFSAEDETMKNNLQTLVQKYSNANSIFDNLVKVLSSTISSCTDTDKLFLHF

SEQ ID NO: 2 - DNA, 999 bp, Shigella flexneri
  source 1..999: mol_type = genomic DNA
atgaatataacaactctgactaatagtatttccacctcatcattcagtccaaacaataccaacggttcatcaaccgaaacagttaattctgatataaaaacaacgaccagttctcatcctgtaagttcccttactatgctcaacgacacccttcataatatcagaacaacaaatcaggcattaaagaaagagctttcacaaaaaacgttgactaaaacatcgctagaagaaatagcattacattcatctcagattagcatggatgtaaataaatccgctcaactattggatattctttccaggaacgaatatccaattaataaagacgcaagagaattattacattcagccccgaaagaagccgagcttgatggagatcaaatgatatctcatagagaactgtgggctaaaattgcaaactccatcaatgatattaatgaacagtatctgaaagtatatgaacatgccgttagttcatatactcaaatgtatcaagattttagcgctgttctttccagtcttgccggctggatctctcccggaggtaacgacggaaactccgtgaaattacaagtcaactcgcttaaaaaggcattggaagaactcaaggaaaaatataaagataaaccgctatatccagcaaataatactgttagtcaggaacaagcaaataaatggcttacagaattaggtggaacaatcggcaaggtatctcaaaaaaacgggggatatgttgtcagtataaacatgaccccaatagacaatatgttaaaaagcttagataatctaggtggaaatggcgaggttgtgctagataatgcaaaatatcaggcatggaatgccggattctctgccgaagatgaaacaatgaaaaataatcttcaaactttagttcaaaaatacagtaatgccaatagtatttttgataatttagtaaaggttttgagtagtacaataagctcatgtacagatacagataaactttttctccatttctga

SEQ ID NO: 3 - protein, 580 aa, Shigella flexneri
MHNVSTTTTGFPLAKILTSTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSNLDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 4 - DNA, 1743 bp, Shigella flexneri
  source 1..1743: mol_type = genomic DNA
atgcataatgtaagcaccacaaccactggttttcctcttgccaaaatattgacttccactgagcttggagacaatactatccaagctgcaaatgatgcagctaacaaattattttctcttacaattgctgatcttactgctaaccaaaatattaatacaactaatgcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtcccagcaacaggcaagacagcaaaaaaacctagaattctccgataaaattaacactcttctatctgaaactgaaggactaaccagagactatgaaaaacaaattaataaactaaaaaacgcagattctaaaataaaagacctagaaaataaaattaaccaaattcaaacaagattatcgaacctcgatccagagtcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctagttgcagcagtcgttctcgtagccactgttggtaaacaggcagcagcaaaacttgcagaaaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtcgacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttga

SEQ ID NO: 5 - DNA, 868 bp, synthetic construct
  source 1..868: mol_type = other DNA
atgattgacatcatgttgcatataggttagataaaacaagtggttatctttccggattgtcttcttgtatgatatataagttttcctcgatgaaaaatataactttcattttttttattttattagcatcgccattatatgcaaatggcgacagattataccgtgctgactctagacccccagatgaaataaaacgtttccggagtcttatgcccagaggtaatgagtacttcgatagaggaactcaaatgaatattaatctttatgatcacgcgagaggaacacaaaccggctttgtcagatatgatgacggatatgtttccacttctcttagtttgagaagtgctcacttagcaggacagtatatattatcaggatattcacttactatatatatcgttatagcaaatatgtttaatgttaatgatgtaattagcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggagattcatcaggaacaatcacaggtgatacttgtaatgaggagacccagaatctgagcacaatatatgccagggaatatcaatcaaaagttaagaggcagatattttcagactatcagtcagaggttgacatatataacagaattcgggatgaattatgaataaagtaaaatgt

SEQ ID NO: 6 - DNA, 604 bp, synthetic construct
  source 1..604: mol_type = other DNA
gttgacatatataacagaattcgggatgaattatgaataaagtaaaatgttatgttttatttacggcgttactatcctctctatatgcacacggagctccccagactattacagaactatgttcggaatatcgcaacacacaaatatatacgataaatgacaagatactatcatatacggaatcgatggcaggcaaaagagaaatggttatcattacatttaagagcggcgaaacatttcaggtcgaagtcccgggcagtcaacatatagactcccagaaaaaagccattgaaaggatgaaggacacattaagaatcacatatctgaccgagaccaaaattgataaattatgtgtatggaataataaaacccccaattcaattgcggcaatcagtatgaaaaactagtttgctttaaaagcatgtctaatgctaggaacctatataacaactactgtacttatactaatgagccttatgctgcatttgaaaaggcggtagaggaggcaataccgatccttaaactgtaacactataacagcttccactacagggagctgttatagcacacagaaaaaactaagctaggctggaggggcaagctt

SEQ ID NO: 7 - DNA, 534 bp, synthetic construct
  source 1..534: mol_type = other DNA
atgggcagcagccatcaccatcatcaccacagccaggatccgatgccaaagtcagccgagcagggcggctccccggcgtcagcttcgcatgaggcgttgcgccatattctcgacgcaggcgcttcgatgggcagcttgcaggggttggacgaggtgcaacagcaggcgttgtacgcgatcgctcatggcgcctacgaacagggccgctatgccgacgcgttgaaaatgttctgcctgctggtcgcgtgcgatccgctggaagcccgttatctgctggccctgggcgccgcggcccaggagctggggctgtacgagcatgccttgcagcaatacgcggccgcggcggctttgcagttggactcccccaggcccctgttgcatggcgccgagtgcctgtatgcgttgggtcgtcgccgcgacgccctggatacgctcgacatggtgcttgagttgtgcgggtcgccggagcatgcggccctgcgcgaacgggccgagtcgctgcgcaggagctatgcacgtgccgactgaaagctt

SEQ ID NO: 8 - protein, 175 aa, synthetic construct
MGSSHHHHHHSQDPMPKSAEQGGSPASASHEALRHILDAGASMGSLQGLDEVQQQALYAIAHGAYEQGRYADALKMFCLLVACDPLEARYLLALGAAAQELGLYEHALQQYAAAAALQLDSPRPLLHGAECLYALGRRRDALDTLDMVLELCGSPEHAALRERAESLRRSYARAD

SEQ ID NO: 9 - protein, 161 aa, synthetic construct
MPKSAEQGGSPASASHEALRHILDAGASMGSLQGLDEVQQQALYAIAHGAYEQGRYADALKMFCLLVACDPLEARYLLALGAAAQELGLYEHALQQYAAAAALQLDSPRPLLHGAECLYALGRRRDALDTLDMVLELCGSPEHAALRERAESLRRSYARAD

SEQ ID NO: 10 - DNA, 544 bp, synthetic construct
  source 1..544: mol_type = other DNA
ccatgggcagcagccatcatcatcatcatcacagcagcggcctggtgccgcgcggcagccatatgctcgagatgtctttaaatatcaccgaaaatgaaagcatctctactgcagtaattgatgcaattaactctggcgctacactgaaagatattaatgcaattcctgatgatatgatggatgacatttattcatatgcttatgacttttacaacaaaggaagaatagaggaagctgaagttttcttcaggtttttatgtatatacgacttttacaatatagactacattatgggactcgcagctatttatcagataaaagaacagttccaacaagcagcagacctttatgctgtcgcttttgcattaggaaaaaatgactatacaccagtattccatactggacaatgccagcttcggttgaaagcccccttaaaagctaaagagtgcttcgaactcgtaattcaacacagcaatgatgaaaaattaaaaataaaagcacaatcatacttggacgcaattcaggatatcaaggagtaggatcc

SEQ ID NO: 11 - protein, 155 aa, synthetic construct
MSLNITENESISTAVIDAINSGATLKDINAIPDDMMDDIYSYAYDFYNKGRIEEAEVFFRFLCIYDFYNVDYIMGLAAIYQIKEQFQQAADLYAVAFALGKNDYTPVFHTGQCQLRLKAPLKAKECFELVIQHSNDEKLKIKAQSYLDAIQDIKE

SEQ ID NO: 12 - DNA, 584 bp, Escherichia coli
  source 1..584: mol_type = genomic DNA
atatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgc

SEQ ID NO: 13 - protein, 194 aa, Escherichia coli
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSR

SEQ ID NO: 14 - protein, 5 aa, synthetic construct
GSAAS

SEQ ID NO: 15 - DNA, 3351 bp, synthetic construct
  source 1..3351: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgaatataacaactctgactaatagtatttccacctcatcattcagtccaaacaataccaacggttcatcaaccgaaacagttaattctgatataaaaacaacgaccagttctcatcctgtaagttcccttactatgctcaacgacacccttcataatatcagaacaacaaatcaggcattaaagaaagagctttcacaaaaaacgttgactaaaacatcgctagaagaaatagcattacattcatctcagattagcatggatgtaaataaatccgctcaactattggatattctttccaggaacgaatatccaattaataaagacgcaagagaattattacattcagccccgaaagaagccgagcttgatggagatcaaatgatatctcatagagaactgtgggctaaaattgcaaactccatcaatgatattaatgaacagtatctgaaagtatatgaacatgccgttagttcatatactcaaatgtatcaagattttagcgctgttctttccagtcttgccggctggatctctcccggaggtaacgacggaaactccgtgaaattacaagtcaactcgcttaaaaaggcattggaagaactcaaggaaaaatataaagataaaccgctatatccagcaaataatactgttagtcaggaacaagcaaataaatggcttacagaattaggtggaacaatcggcaaggtatctcaaaaaaacgggggatatgttgtcagtataaacatgaccccaatagacaatatgttaaaaagcttagataatctaggtggaaatggcgaggttgtgctagataatgcaaaatatcaggcatggaatgccggattctctgccgaagatgaaacaatgaaaaataatcttcaaactttagttcaaaaatacagtaatgccaatagtatttttgataatttagtaaaggttttgagtagtacaataagctcatgtacagatacagataaactttttctccatttcctcgagatgcataatgtaagcaccacaaccactggttttcctcttgccaaaatattgacttccactgagcttggagacaatactatccaagctgcaaatgatgcagctaacaaattattttctcttacaattgctgatcttactgctaaccaaaatattaatacaactaatgcacactcaacttcaaatatattaatccctgaacttaaagcaccaaagtcattaaatgcaagttcccaactaacgcttttaattggaaaccttattcaaatactcggtgaaaaatctttaactgcattaacaaataaaattactgcttggaagtcccagcaacaggcaagacagcaaaaaaacctagaattctccgataaaattaacactcttctatctgaaactgaaggactaaccagagactatgaaaaacaaattaataaactaaaaaacgcagattctaaaataaaagacctagaaaataaaattaaccaaattcaaacaagattatcgaacctcgatccagagtcaccagaaaagaaaaaattaagccgggaagaaatacaactcactatcaaaaaagacgcagcagttaaagacaggacattgattgagcagaaaaccctgtcaattcatagcaaacttacagataaatcaatgcaactcgaaaaagaaatagactctttttctgcattttcaaacacagcatctgctgaacagctatcaacccagcagaaatcattaaccggacttgccagtgttactcaattgatggcaacctttattcaactagttggaaaaaataatgaagaatctttaaaaaatgatctggctctattccagtctctccaagaatcaagaaaaactgaaatggagagaaaatctgatgagtatgctgctgaagtacgtaaagcagaagaactcaacagagtaatgggttgtgttgggaaaatacttggggcacttttaactatcgttagtgttgttgcagcagctttttctggaggagcctctctagcactggcagctgttggtttagctcttatggttacggatgctatagtacaagcagcgaccggcaattccttcatggaacaagccctgaatccgatcatgaaagcagtcattgaacccttaatcaaactcctttcagatgcatttacaaaaatgctcgaaggcttgggcgtcgactcgaaaaaagccaaaatgattggctctattctgggggcaatcgcaggcgctcttgtcctagttgcagcagtcgttctcgtagccactgttggtaaacaggcagcagcaaaacttgcagaaaatattggcaaaataataggtaaaaccctcacagaccttataccaaagtttctcaagaatttttcttctcaactggacgatttaatcactaatgctgttgccagattaaataaatttcttggtgcagcgggtgatgaagtaatatccaaacaaattatttccacccatttaaaccaagcagttttattaggagaaagtgttaactctgccacacaagcgggaggaagtgtcgcttctgctgttttccagaacagcgcgtcgacaaatctagcagacctgacattatcgaaatatcaagttgaacaactgtcaaaatatatcagtgaagcaatagaaaaattcggccaattgcaggaagtaattgcagatctattagcctcaatgtccaactctcaggctaatagaactgatgttgcaaaagcaattttgcaacaaactactgcttgaggatcc

SEQ ID NO: 16 - protein, 1081 aa, synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMNITTLTNSISTSSFSPNNTNGSSTETVNSDIKTTTSSHPSSLTMLNDTLHNIRTTNQALKKELSQKTLRNEYPINKDARELLHSAPKEAELDGDQMISHRELWAKIANSINDINEQYLKVYEHAVSSYTQMYQDFSAVLSSLAGWISPGGNDGNSVKLQVNSLKKALEELKEKYKDKPLYPANNTVSQEQANKWLTELGGTIGKVSQKNGGYVVSINMTPIDNMLKSLDNLGGNGEVVLDNAKYQAWNGFSAEDETMKNNLQTLVQKYSNANSIFDNLVKVLSSTISSCTDTDKLFLHFLEMHNVSTTTTGFPLAKILTSTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSNLDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 17 - DNA, 552 bp, synthetic construct
  source 1..552: mol_type = other DNA
atgggcagcagccatcaccatcatcaccacagccaggatccgatgaaccagccgaccccttccgacaccgaccagcaacaggcgctggaggccttcctgcgcgacggcggcaccctggcgatgcttcgcggactcagcgaggacaccctggagcagctctatgcgctgggcttcaaccagtaccaggcgggcaagtgggacgacgcgcagaagatcttccaggcactgtgcatgctcgaccactacgacgcccgctactttctcggcctgggcgcctgccgccagtccctcggtctctatgaacaggccctgcagagctacagctacggcgcgctgatggacatcaacgagccgcgctttcccttccatgccgccgagtgccacctgcaactgggtgatctcgacggagccgagagtggcttctactcggcccgggccctggccgcggcacagccggcgcacgaggccctggccgcgcgtgccggcgccatgttggaagccgtaaccgcgagaaaggatcgagcctatgaatccgataacgcttgaaagctt

SEQ ID NO: 18 - protein, 181 aa, synthetic construct
MGSSHHHHHHSQDPMNQPTPSDTDQQQALEAFLRDGGTLAMLRGLSEDTLEQLYALGFNQYQAGKWDDAQKIFQALCMLDHYDARYFLGLGACRQSLGLYEQALQSYSYGALMDINEPRFPFHAAECHLQLGDLDGAESGFYSARALAAAQPAHEALAARAGAMLEAVTARKDRAYESDNA

SEQ ID NO: 19 - DNA, 546 bp, synthetic construct
  source 1..546: mol_type = other DNA
atgggcagcagccatcaccatcatcaccacagccaggatccgatggactaccagaacaacgtcagcgaagaacgtgttgcggaaatgatttgggatgccgttagtgaaggcgccacgctaaaagacgttcatggaatccctcaagatatgatggacggtttatatgctcatgcttatgagttttataaccagggacgactggatgaagctgagacgttctttcgtttcttatgcatttatgatttttacaatcccgattacaccatgggactggcggcagtatgccaactgaaaaaacaatttcagaaagcatgtgacctttatgcagtagcgtttacgttacttaaaaatgattatcgccccgttttttttaccgggcagtgtcaattattaatgcgtaaggcagcaaaagccagacagtgttttgaacttgtcaatgaacgtactgaagatgagtctctgcgggcaaaagcgttggtctatctggaggcgctaaaaacggcggagacagagcagcacagcgagcaggagaaggagtaaaagctt

SEQ ID NO: 20 - protein, 179 aa, synthetic construct
MGSSHHHHHHSQDPMDYQNNVSEERVAEMIWDAVSEGATLKDVHGIPQDMMDGLYAHAYEFYNQGRLDEAETFFRFLCIYDFYNPDYTMGLAAVCQLKKQFQKACDLYAVAFTLLKNDYRPVFFTGQCQLLMRKAAKARQCFELVNERTEDESLRAKALVYLEALKTAETEQHSEQEKE

SEQ ID NO: 21 - DNA, 1029 bp, Salmonella enterica
  source 1..1029: mol_type = genomic DNA
atgcttaatattcaaaattattccgcttctcctcatccggggatcgttgccgaacggccgcagactccctcggcgagcgagcacgtcgagactgccgtggtaccgtctaccacagaacatcgcggtacagatatcatttcattatcgcaggcggctactaaaatccaccaggcacagcagacgctgcagtcaacgccaccgatctctgaagagaataatgacgagcgcacgctggcgcgccagcagttgaccagcagcctgaatgcgctggcgaagtccggcgtgtcattatccgcagaacaaaatgagaacctgcggagcgcgttttctgcgccgacgtcggccttatttagcgcttcgcctatggcgcagccgagaacaaccatttctgatgctgagatttgggatatggtttcccaaaatatatcggcgataggtgacagctatctgggcgtttatgaaaacgttgtcgcagtctataccgatttttatcaggccttcagtgatattctttccaaaatgggaggctggttattaccaggtaaggacggtaataccgttaagctagatgttacctcactcaaaaatgatttaaacagtttagtcaataaatataatcaaataaacagtaataccgttttatttccagcgcagtcaggcagcggcgttaaagtagccactgaagcggaagcgagacagtggctcagtgaattgaatttaccgaatagctgcctgaaatcttatggatccggttatgtcgtcaccgttgatctgacgccattacaaaaaatggttcaggatattgatggtttaggcgcgccgggaaaagactcaaaactcgaaatggataacgccaaatatcaagcctggcagtcgggttttaaagcgcaggaagaaaatatgaaaaccacattacagacgctgacgcaaaaatatagcaatgccaattcattgtacgacaacctggtaaaagtgctgagcagtacgataagtagcagcctggaaaccgccaaaagcttcctgcaagga

SEQ ID NO: 22 - protein, 343 aa, Salmonella enterica
MLNIQNYSASPHPGIVAERPQTPSASEHVETAVVPSTTEHRGTDIISLSQAATKIHQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQG

SEQ ID NO: 23 - DNA, 1782 bp, Salmonella enterica
  source 1..1782: mol_type = genomic DNA
atggtaaatgacgcaagtagcattagccgtagcggatatacccaaaatccgcgcctcgctgaggcggcttttgaaggcgttcgtaagaacacggactttttaaaagcggcggataaagcttttaaagatgtggtggcaacgaaagcgggcgaccttaaagccggaacaaagtccggcgagagcgctattaatacggtgggtctaaagccgcctacggacgccgcccgggaaaaactctccagcgaagggcaattgacattactgcttggcaagttaatgaccctactgggcgatgtttcgctgtctcaactggagtctcgtctggcggtatggcaggcgatgattgagtcacaaaaagagatggggattcaggtatcgaaagaattccagacggctctgggagaggctcaggaggcgacggatctctatgaagccagtatcaaaaagacggataccgccaagagtgtttatgacgctgcgaccaaaaaactgacgcaggcgcaaaataaattgcaatcgctggacccggctgaccccggctatgcacaagctgaagccgcggtagaacaggccggaaaagaagcgacagaggcgaaagaggccttagataaggccacggatgcgacggttaaagcaggcacagacgccaaagcgaaagccgagaaagcggataacattctgaccaaattccagggaacggctaatgccgcctctcagaatcaggtttcccagggtgagcaggataatctgtcaaatgtcgcccgcctcactatgctcatggccatgtttattgagattgtgggcaaaaatacggaagaaagcctgcaaaacgatcttgcgcttttcaacgccttgcaggaagggcgtcaggcggagatggaaaagaaatcggctgaattccaggaagagacgcgcaaagccgaggaaacgaaccgcattatgggatgtatcgggaaagtcctcggcgcgctgctaaccattgtcagcgttgtggccgctgtttttaccggtggggcgagtctggcgctggctgcggtgggacttgcggtaatggtggccgatgaaattgtgaaggcggcgacgggagtgtcgtttattcagcaggcgctaaacccgattatggagcatgtgctgaagccgttaatggagctgattggcaaggcgattaccaaagcgctggaaggattaggcgtcgataagaaaacggcagagatggccggcagcattgttggtgcgattgtcgccgctattgccatggtggcggtcattgtggtggtcgcagttgtcgggaaaggcgcggcggcgaaactgggtaacgcgctgagcaaaatgatgggcgaaacgattaagaagttggtgcctaacgtgctgaaacagttggcgcaaaacggcagcaaactctttacccaggggatgcaacgtattactagcggtctgggtaatgtgggtagcaagatgggcctgcaaacgaatgccttaagtaaagagctggtaggtaataccctaaataaagtggcgttgggcatggaagtcacgaataccgcagcccagtcagccggtggtgttgccgagggcgtatttattaaaaatgccagcgaggcgcttgctgattttatgctcgcccgttttgccatggatcagattcagcagtggcttaaacaatccgtagaaatatttggtgaaaaccagaaggtaacggcggaactgcaaaaagccatgtcttctgcggtacagcaaaatgcggatgcttcgcgttttattctgcgccagagtcgcgcataa

SEQ ID NO: 24 - protein, 593 aa, Salmonella enterica
MVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 25 - DNA, 2817 bp, Salmonella enterica
  source 1..2817: mol_type = genomic DNA
atgcttaatattcaaaattattccgcttctcctcatccggggatcgttgccgaacggccgcagactccctcggcgagcgagcacgtcgagactgccgtggtaccgtctaccacagaacatcgcggtacagatatcatttcattatcgcaggcggctactaaaatccaccaggcacagcagacgctgcagtcaacgccaccgatctctgaagagaataatgacgagcgcacgctggcgcgccagcagttgaccagcagcctgaatgcgctggcgaagtccggcgtgtcattatccgcagaacaaaatgagaacctgcggagcgcgttttctgcgccgacgtcggccttatttagcgcttcgcctatggcgcagccgagaacaaccatttctgatgctgagatttgggatatggtttcccaaaatatatcggcgataggtgacagctatctgggcgtttatgaaaacgttgtcgcagtctataccgatttttatcaggccttcagtgatattctttccaaaatgggaggctggttattaccaggtaaggacggtaataccgttaagctagatgttacctcactcaaaaatgatttaaacagtttagtcaataaatataatcaaataaacagtaataccgttttatttccagcgcagtcaggcagcggcgttaaagtagccactgaagcggaagcgagacagtggctcagtgaattgaatttaccgaatagctgcctgaaatcttatggatccggttatgtcgtcaccgttgatctgacgccattacaaaaaatggttcaggatattgatggtttaggcgcgccgggaaaagactcaaaactcgaaatggataacgccaaatatcaagcctggcagtcgggttttaaagcgcaggaagaaaatatgaaaaccacattacagacgctgacgcaaaaatatagcaatgccaattcattgtacgacaacctggtaaaagtgctgagcagtacgataagtagcagcctggaaaccgccaaaagcttcctgcaaggagtcgacatggtaaatgacgcaagtagcattagccgtagcggatatacccaaaatccgcgcctcgctgaggcggcttttgaaggcgttcgtaagaacacggactttttaaaagcggcggataaagcttttaaagatgtggtggcaacgaaagcgggcgaccttaaagccggaacaaagtccggcgagagcgctattaatacggtgggtctaaagccgcctacggacgccgcccgggaaaaactctccagcgaagggcaattgacattactgcttggcaagttaatgaccctactgggcgatgtttcgctgtctcaactggagtctcgtctggcggtatggcaggcgatgattgagtcacaaaaagagatggggattcaggtatcgaaagaattccagacggctctgggagaggctcaggaggcgacggatctctatgaagccagtatcaaaaagacggataccgccaagagtgtttatgacgctgcgaccaaaaaactgacgcaggcgcaaaataaattgcaatcgctggacccggctgaccccggctatgcacaagctgaagccgcggtagaacaggccggaaaagaagcgacagaggcgaaagaggccttagataaggccacggatgcgacggttaaagcaggcacagacgccaaagcgaaagccgagaaagcggataacattctgaccaaattccagggaacggctaatgccgcctctcagaatcaggtttcccagggtgagcaggataatctgtcaaatgtcgcccgcctcactatgctcatggccatgtttattgagattgtgggcaaaaatacggaagaaagcctgcaaaacgatcttgcgcttttcaacgccttgcaggaagggcgtcaggcggagatggaaaagaaatcggctgaattccaggaagagacgcgcaaagccgaggaaacgaaccgcattatgggatgtatcgggaaagtcctcggcgcgctgctaaccattgtcagcgttgtggccgctgtttttaccggtggggcgagtctggcgctggctgcggtgggacttgcggtaatggtggccgatgaaattgtgaaggcggcgacgggagtgtcgtttattcagcaggcgctaaacccgattatggagcatgtgctgaagccgttaatggagctgattggcaaggcgattaccaaagcgctggaaggattaggcgtcgataagaaaacggcagagatggccggcagcattgttggtgcgattgtcgccgctattgccatggtggcggtcattgtggtggtcgcagttgtcgggaaaggcgcggcggcgaaactgggtaacgcgctgagcaaaatgatgggcgaaacgattaagaagttggtgcctaacgtgctgaaacagttggcgcaaaacggcagcaaactctttacccaggggatgcaacgtattactagcggtctgggtaatgtgggtagcaagatgggcctgcaaacgaatgccttaagtaaagagctggtaggtaataccctaaataaagtggcgttgggcatggaagtcacgaataccgcagcccagtcagccggtggtgttgccgagggcgtatttattaaaaatgccagcgaggcgcttgctgattttatgctcgcccgttttgccatggatcagattcagcagtggcttaaacaatccgtagaaatatttggtgaaaaccagaaggtaacggcggaactgcaaaaagccatgtcttctgcggtacagcaaaatgcggatgcttcgcgttttattctgcgccagagtcgcgcataa

SEQ ID NO: 26 - protein, 938 aa, Salmonella enterica
MLNIQNYSASPHPGIVAERPQTPSASEHVETAVVPSTTEHRGTDIISLSQAATKIHQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQGVDMVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 27 - DNA, 3423 bp, synthetic construct
  source 1..3423: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgcttaatattcaaaattattccgcttctcctcatccggggatcgttgccgaacggccgcagactccctcggcgagcgagcacgtcgagactgccgtggtaccgtctaccacagaacatcgcggtacagatatcatttcattatcgcaggcggctactaaaatccaccaggcacagcagacgctgcagtcaacgccaccgatctctgaagagaataatgacgagcgcacgctggcgcgccagcagttgaccagcagcctgaatgcgctggcgaagtccggcgtgtcattatccgcagaacaaaatgagaacctgcggagcgcgttttctgcgccgacgtcggccttatttagcgcttcgcctatggcgcagccgagaacaaccatttctgatgctgagatttgggatatggtttcccaaaatatatcggcgataggtgacagctatctgggcgtttatgaaaacgttgtcgcagtctataccgatttttatcaggccttcagtgatattctttccaaaatgggaggctggttattaccaggtaaggacggtaataccgttaagctagatgttacctcactcaaaaatgatttaaacagtttagtcaataaatataatcaaataaacagtaataccgttttatttccagcgcagtcaggcagcggcgttaaagtagccactgaagcggaagcgagacagtggctcagtgaattgaatttaccgaatagctgcctgaaatcttatggatccggttatgtcgtcaccgttgatctgacgccattacaaaaaatggttcaggatattgatggtttaggcgcgccgggaaaagactcaaaactcgaaatggataacgccaaatatcaagcctggcagtcgggttttaaagcgcaggaagaaaatatgaaaaccacattacagacgctgacgcaaaaatatagcaatgccaattcattgtacgacaacctggtaaaagtgctgagcagtacgataagtagcagcctggaaaccgccaaaagcttcctgcaaggagtcgacatggtaaatgacgcaagtagcattagccgtagcggatatacccaaaatccgcgcctcgctgaggcggcttttgaaggcgttcgtaagaacacggactttttaaaagcggcggataaagcttttaaagatgtggtggcaacgaaagcgggcgaccttaaagccggaacaaagtccggcgagagcgctattaatacggtgggtctaaagccgcctacggacgccgcccgggaaaaactctccagcgaagggcaattgacattactgcttggcaagttaatgaccctactgggcgatgtttcgctgtctcaactggagtctcgtctggcggtatggcaggcgatgattgagtcacaaaaagagatggggattcaggtatcgaaagaattccagacggctctgggagaggctcaggaggcgacggatctctatgaagccagtatcaaaaagacggataccgccaagagtgtttatgacgctgcgaccaaaaaactgacgcaggcgcaaaataaattgcaatcgctggacccggctgaccccggctatgcacaagctgaagccgcggtagaacaggccggaaaagaagcgacagaggcgaaagaggccttagataaggccacggatgcgacggttaaagcaggcacagacgccaaagcgaaagccgagaaagcggataacattctgaccaaattccagggaacggctaatgccgcctctcagaatcaggtttcccagggtgagcaggataatctgtcaaatgtcgcccgcctcactatgctcatggccatgtttattgagattgtgggcaaaaatacggaagaaagcctgcaaaacgatcttgcgcttttcaacgccttgcaggaagggcgtcaggcggagatggaaaagaaatcggctgaattccaggaagagacgcgcaaagccgaggaaacgaaccgcattatgggatgtatcgggaaagtcctcggcgcgctgctaaccattgtcagcgttgtggccgctgtttttaccggtggggcgagtctggcgctggctgcggtgggacttgcggtaatggtggccgatgaaattgtgaaggcggcgacgggagtgtcgtttattcagcaggcgctaaacccgattatggagcatgtgctgaagccgttaatggagctgattggcaaggcgattaccaaagcgctggaaggattaggcgtcgataagaaaacggcagagatggccggcagcattgttggtgcgattgtcgccgctattgccatggtggcggtcattgtggtggtcgcagttgtcgggaaaggcgcggcggcgaaactgggtaacgcgctgagcaaaatgatgggcgaaacgattaagaagttggtgcctaacgtgctgaaacagttggcgcaaaacggcagcaaactctttacccaggggatgcaacgtattactagcggtctgggtaatgtgggtagcaagatgggcctgcaaacgaatgccttaagtaaagagctggtaggtaataccctaaataaagtggcgttgggcatggaagtcacgaataccgcagcccagtcagccggtggtgttgccgagggcgtatttattaaaaatgccagcgaggcgcttgctgattttatgctcgcccgttttgccatggatcagattcagcagtggcttaaacaatccgtagaaatatttggtgaaaaccagaaggtaacggcggaactgcaaaaagccatgtcttctgcggtacagcaaaatgcggatgcttcgcgttttattctgcgccagagtcgcgcataactcgag

SEQ ID NO: 28 - protein, 1137 aa, synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMLNIQNYSASPHPGIVAERPQTPSASEHVETAVVPSTTEHRGTDIISLSQAATKIHQAQQTLQSTPPISEENNDERTLARQQLTSSLNALAKSGVSLSAEQNENLRSAFSAPTSALFSASPMAQPRTTISDAEIWDMVSQNISAIGDSYLGVYENVVAVYTDFYQAFSDILSKMGGWLLPGKDGNTVKLDVTSLKNDLNSLVNKYNQINSNTVLFPAQSGSGVKVATEAEARQWLSELNLPNSCLKSYGSGYVVTVDLTPLQKMVQDIDGLGAPGKDSKLEMDNAKYQAWQSGFKAQEENMKTTLQTLTQKYSNANSLYDNLVKVLSSTISSSLETAKSFLQGVDMVNDASSISRSGYTQNPRLAEAAFEGVRKNTDFLKAADKAFKDVVATKAGDLKAGTKSGESAINTVGLKPPTDAAREKLSSEGQLTLLLGKLMTLLGDVSLSQLESRLAVWQAMIESQKEMGIQVSKEFQTALGEAQEATDLYEASIKKTDTAKSVYDAATKKLTQAQNKLQSLDPADPGYAQAEAAVEQAGKEATEAKEALDKATDATVKAGTDAKAKAEKADNILTKFQGTANAASQNQVSQGEQDNLSNVARLTMLMAMFIEIVGKNTEESLQNDLALFNALQEGRQAEMEKKSAEFQEETRKAEETNRIMGCIGKVLGALLTIVSVVAAVFTGGASLALAAVGLAVMVADEIVKAATGVSFIQQALNPIMEHVLKPLMELIGKAITKALEGLGVDKKTAEMAGSIVGAIVAAIAMVAVIVVVAVVGKGAAAKLGNALSKMMGETIKKLVPNVLKQLAQNGSKLFTQGMQRITSGLGNVGSKMGLQTNALSKELVGNTLNKVALGMEVTNTAAQSAGGVAEGVFIKNASEALADFMLARFAMDQIQQWLKQSVEIFGENQKVTAELQKAMSSAVQQNADASRFILRQSRA

SEQ ID NO: 29 - DNA, 522 bp, synthetic construct
  source 1..522: mol_type = other DNA
atgggcagcagccatcaccatcatcaccacagccaggatccgatgaaaaaagacccgaccctacaacaggcacatgacacgatgcggtttttccggcgtggcggctcgctgcgtatgttgttggatgacgatgttacacagccgcttaatactctgtatcgctatgccacgcagcttatggaggtaaaagaattcgccggcgcagcgcgactttttcaattgctgacgatatatgatgcctggtcatttgactactggtttcggttaggggaatgctgccaggctcaaaaacattggggggaagcgatatacgcttatggacgcgcggcacaaattaagattgatgcgccgcaggcgccatgggccgcagcggaatgctatctcgcgtgtgataacgtctgttatgcaatcaaagcgttaaaggccgtggtgcgtatttgcggcgaggtcagtgaacatcaaattctccgacagcgtgcagaaaagatgttacagcaactttctgacaggagctaaaagctt

SEQ ID NO: 30 - protein, 171 aa, synthetic construct
MGSSHHHHHHSQDPMKKDPTLQQAHDTMRFFRRGGSLRMLLDDDVTQPLNTLYRYATQLMEVKEFAGAARLFQLLTIYDAWSFDYWFRLGECCQAQKHWGEAIYAYGRAAQIKIDAPQAPWAAAECYLACDNVCYAIKALKAVVRICGEVSEHQILRQRAEKMLQQLSDRS

SEQ ID NO: 31 - DNA, 588 bp, Salmonella enterica
  source 1..588: mol_type = genomic DNA
atgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactca

SEQ ID NO: 32 - protein, 196 aa, Salmonella enterica
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 33 - DNA, 1455 bp, Salmonella enterica
  source 1..1455: mol_type = genomic DNA
atgaatcgaattcacagtaatagcgacagcgccgcaggagtaaccgccttaacacatcatcacttaagcaatgtcagttgcgtttcctcgggttcgctgggaaagcgccagcatcgtgtgaattctacttttggcgatggcaacgccgcgtgtctgctatccgggaaaattagtcttcaggaggcaagcaatgcgttgaagcaactgcttgatgccgtacccggaaatcataagcgtccatcattgcctgactttttgcagaccaatcccgcggttttatcaatgatgatgacgtcattaatactcaacgtctttggtaataacgctcaatcgttatgccaacagcttgagcgggcaactgaggtgcaaaatgcattacgtaataagcaggtaaaggagtatcaggagcagatccagaaagcgatagagcaggaggataaagcgcgtaaagcgggtatttttggcgctatttttgactggattaccggcatatttgaaaccgtgattggcgccttaaaagttgtggaaggttttctgtccggaaatcccgcagaaatggctagcggcgtagcttatatggccgcaggttgtgcaggaatggttaaagccggagccgaaacggcaatgatgtgcggtgctgaccacgatacctgtcaggcaattattgacgtgacaagtaagattcaatttggttgtgaagccgtcgcgctggcactggatgttttccagattggccgtgcttttatggcgacgagaggtttatctggcgcagctgcaaaagtgcttgactccggttttggcgaggaagtggttgagcgtatggtaggtgcaggggaagcagaaatagaggagttggctgaaaagtttggcgaagaagtgagcgaaagtttttccaaacaatttgagccgcttgaacgtgaaatggctatggcgaatgagatggcagaggaggctgccgagttttctcgtaacgtagaaaataatatgacgcgaagcgcgggaaaaagctttacgaaagagggggtgaaagcaatggcaaaagaagcggcaaaagaagccctggaaaaatgtgtgcaagaaggtggaaagttcctgttaaaaaaattccgtaataaagttctcttcaatatgttcaaaaaaatcctgtatgccttactgagggattgttcatttaaaggcttacaggctatcagatgtgcaaccgagggcgccagtcagatgaatactggcatggttaacacagaaaaagcgaagatcgaaaagaaaatagagcaattaataactcagcaacggtttctggatttcataatgcaacaaacagaaaaccagaaaaagatagaacaaaaacgcttagaggagctttataaggggagcggtgccgcgcttagagatgtattagataccattgatcactatagtagcgttcaggcgagaatagctggctatcgcgcttaa

SEQ ID NO: 34 - protein, 484 aa, Salmonella enterica
MNRIHSNSDSAAGVTALTHHHLSNVSCVSSGSLGKRQHRVNSTFGDGNAACLLSGKISLQEASNALKQLLDAVPGNHKRPSLPDFLQTNPAVLSMMMTSLILNVFGNNAQSLCQQLERATEVQNALRNKQVKEYQEQIQKAIEQEDKARKAGIFGAIFDWITGIFETVIGALKVVEGFLSGNPAEMASGVAYMAAGCAGMVKAGAETAMMCGADHDTCQAIIDVTSKIQFGCEAVALALDVFQIGRAFMATRGLSGAAAKVLDSGFGEEVVERMVGAGEAEIEELAEKFGEEVSESFSKQFEPLEREMAMANEMAEEAAEFSRNVENNMTRSAGKSFTKEGVKAMAKEAAKEALEKCVQEGGKFLLKKFRNKVLFNMFKKILYALLRDCSFKGLQAIRCATEGASQMNTGMVNTEKAKIEKKIEQLITQQRFLDFIMQQTENQKKIEQKRLEELYKGSGAALRDVLDTIDHYSSVQARIAGYRA

SEQ ID NO: 35 - DNA, 2049 bp, Salmonella enterica
  source 1..2049: mol_type = genomic DNA
atgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcagagctcatgaatcgaattcacagtaatagcgacagcgccgcaggagtaaccgccttaacacatcatcacttaagcaatgtcagttgcgtttcctcgggttcgctgggaaagcgccagcatcgtgtgaattctacttttggcgatggcaacgccgcgtgtctgctatccgggaaaattagtcttcaggaggcaagcaatgcgttgaagcaactgcttgatgccgtacccggaaatcataagcgtccatcattgcctgactttttgcagaccaatcccgcggttttatcaatgatgatgacgtcattaatactcaacgtctttggtaataacgctcaatcgttatgccaacagcttgagcgggcaactgaggtgcaaaatgcattacgtaataagcaggtaaaggagtatcaggagcagatccagaaagcgatagagcaggaggataaagcgcgtaaagcgggtatttttggcgctatttttgactggattaccggcatatttgaaaccgtgattggcgccttaaaagttgtggaaggttttctgtccggaaatcccgcagaaatggctagcggcgtagcttatatggccgcaggttgtgcaggaatggttaaagccggagccgaaacggcaatgatgtgcggtgctgaccacgatacctgtcaggcaattattgacgtgacaagtaagattcaatttggttgtgaagccgtcgcgctggcactggatgttttccagattggccgtgcttttatggcgacgagaggtttatctggcgcagctgcaaaagtgcttgactccggttttggcgaggaagtggttgagcgtatggtaggtgcaggggaagcagaaatagaggagttggctgaaaagtttggcgaagaagtgagcgaaagtttttccaaacaatttgagccgcttgaacgtgaaatggctatggcgaatgagatggcagaggaggctgccgagttttctcgtaacgtagaaaataatatgacgcgaagcgcgggaaaaagctttacgaaagagggggtgaaagcaatggcaaaagaagcggcaaaagaagccctggaaaaatgtgtgcaagaaggtggaaagttcctgttaaaaaaattccgtaataaagttctcttcaatatgttcaaaaaaatcctgtatgccttactgagggattgttcatttaaaggcttacaggctatcagatgtgcaaccgagggcgccagtcagatgaatactggcatggttaacacagaaaaagcgaagatcgaaaagaaaatagagcaattaataactcagcaacggtttctggatttcataatgcaacaaacagaaaaccagaaaaagatagaacaaaaacgcttagaggagctttataaggggagcggtgccgcgcttagagatgtattagataccattgatcactatagtagcgttcaggcgagaatagctggctatcgcgcttaa

SEQ ID NO: 36 - protein, 682 aa, Salmonella enterica
MSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYSELMNRIHSNSDSAAGVTALTHHHLSNVSCVSSGSLGKRQHRVNSTFGDGNAACLLSGKISLQEASNALKQLLDAVPGNHKRPSLPDFLQTNPAVLSMMMTSLILNVFGNNAQSLCQQLERATEVQNALRNKQVKEYQEQIQKAIEQEDKARKAGIFGAIFDWITGIFETVIGALKVVEGFLSGNPAEMASGVAYMAAGCAGMVKAGAETAMMCGADHDTCQAIIDVTSKIQFGCEAVALALDVFQIGRAFMATRGLSGAAAKVLDSGFGEEVVERMVGAGEAEIEELAEKFGEEVSESFSKQFEPLEREMAMANEMAEEAAEFSRNVENNMTRSAGKSFTKEGVKAMAKEAAKEALEKCVQEGGKFLLKKFRNKVLFNMFKKILYALLRDCSFKGLQAIRCATEGASQMNTGMVNTEKAKIEKKIEQLITQQRFLDFIMQQTENQKKIEQKRLEELYKGSGAALRDVLDTIDHYSSVQARIAGYRA

SEQ ID NO: 37 - DNA, 2655 bp, synthetic construct
  source 1..2655: mol_type = other DNA
catatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcagagctcatgaatcgaattcacagtaatagcgacagcgccgcaggagtaaccgccttaacacatcatcacttaagcaatgtcagttgcgtttcctcgggttcgctgggaaagcgccagcatcgtgtgaattctacttttggcgatggcaacgccgcgtgtctgctatccgggaaaattagtcttcaggaggcaagcaatgcgttgaagcaactgcttgatgccgtacccggaaatcataagcgtccatcattgcctgactttttgcagaccaatcccgcggttttatcaatgatgatgacgtcattaatactcaacgtctttggtaataacgctcaatcgttatgccaacagcttgagcgggcaactgaggtgcaaaatgcattacgtaataagcaggtaaaggagtatcaggagcagatccagaaagcgatagagcaggaggataaagcgcgtaaagcgggtatttttggcgctatttttgactggattaccggcatatttgaaaccgtgattggcgccttaaaagttgtggaaggttttctgtccggaaatcccgcagaaatggctagcggcgtagcttatatggccgcaggttgtgcaggaatggttaaagccggagccgaaacggcaatgatgtgcggtgctgaccacgatacctgtcaggcaattattgacgtgacaagtaagattcaatttggttgtgaagccgtcgcgctggcactggatgttttccagattggccgtgcttttatggcgacgagaggtttatctggcgcagctgcaaaagtgcttgactccggttttggcgaggaagtggttgagcgtatggtaggtgcaggggaagcagaaatagaggagttggctgaaaagtttggcgaagaagtgagcgaaagtttttccaaacaatttgagccgcttgaacgtgaaatggctatggcgaatgagatggcagaggaggctgccgagttttctcgtaacgtagaaaataatatgacgcgaagcgcgggaaaaagctttacgaaagagggggtgaaagcaatggcaaaagaagcggcaaaagaagccctggaaaaatgtgtgcaagaaggtggaaagttcctgttaaaaaaattccgtaataaagttctcttcaatatgttcaaaaaaatcctgtatgccttactgagggattgttcatttaaaggcttacaggctatcagatgtgcaaccgagggcgccagtcagatgaatactggcatggttaacacagaaaaagcgaagatcgaaaagaaaatagagcaattaataactcagcaacggtttctggatttcataatgcaacaaacagaaaaccagaaaaagatagaacaaaaacgcttagaggagctttataaggggagcggtgccgcgcttagagatgtattagataccattgatcactatagtagcgttcaggcgagaatagctggctatcgcgcttaactcgag

SEQ ID NO: 38 - protein, 881 aa, synthetic construct
MDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYSELMNRIHSNSDSAAGVTALTHHHLSNVSCVSSGSLGKRQHRVNSTFGDGNAACLLSGKISLQEASNALKQLLDAVPGNHKRPSLPDFLQTNPAVLSMMMTSLILNVFGNNAQSLCQQLERATEVQNALRNKQVKEYQEQIQKAIEQEDKARKAGIFGAIFDWITGIFETVIGALKVVEGFLSGNPAEMASGVAYMAAGCAGMVKAGAETAMMCGADHDTCQAIIDVTSKIQFGCEAVALALDVFQIGRAFMATRGLSGAAAKVLDSGFGEEVVERMVGAGEAEIEELAEKFGEEVSESFSKQFEPLEREMAMANEMAEEAAEFSRNVENNMTRSAGKSFTKEGVKAMAKEAAKEALEKCVQEGGKFLLKKFRNKVLFNMFKKILYALLRDCSFKGLQAIRCATEGASQMNTGMVNTEKAKIEKKIEQLITQQRFLDFIMQQTENQKKIEQKRLEELYKGSGAALRDVLDTIDHYSSVQARIAGYRA

SEQ ID NO: 39 - DNA, 1254 bp, synthetic construct
  source 1..1254: mol_type = other DNA
atgggcagcagccatcatcatcatcatcacagcagcggcctggtgccgcgcggcagccatatggacaatggcgatcgtttataccgtgccgactcgcgtcccccagatgagattaaacgtagcggtgggttaatgccacgtgggcacaatgagtattttgaccgtggaacacagatgaacattaacctttacgatcatgcccgtgggacccagaccgggtttgtccgttatgatgacgggtatgttagtacgagtttgtccttacgctccgcacaccttgcgggacaaagtattttatcaggctacagcacatattacatttatgtgatcgccactgccccaaacatgttcaatgtgaacgatgtgttgggggtttacagcccccatccatatgaacaagaagtctcggcccttggggggatcccatatagccagatttatggttggtaccgcgtaaattttggtgtgattgatgaacgtttgcatcgtaaccgtgaataccgcgatcgctactaccgtaacttgaacattgcacctgccgaggacggctatcgtttagcgggattcccacccgatcatcaggcgtggcgtgaggaaccgtggatccatcacgcccctcaggggtgcgggaacagtagtcgcgggtccgcggcatccatgtcttcaggaaacatcttatggggaagtcaaaaccctattgtgtttaaaaatagcttcggcgtcagcaacgctgataccgggagccaggatgacttatcccagcaaaatccgtttgccgaagggtatggtgttttgcttattctccttatggttattcaggctatcgcaaataataaatttattgaagtccagaagaacgctgaacgtgccagaaatacccaggaaaagtcaaatgagatggatgaggtgattgctaaagcagccaaaggggatgctaaaaccaaagaggaggtgcctgaggatgtaattaaatacatgcgtgataatggtattctcatcgatggtatgaccattgatgattatatggctaaatatggcgatcatgggaagctggataaaggtggcctacaggcgatcaaagcggctttggataatgacgccaaccggaataccgatcttatgagtcaggggcagataacaattcaaaaaatgtctcaggagcttaacgctgtccttacccaactgacagggcttatcagtaagtggggggaaatttccagtatgatagcgcagaaaacgtactcataaggatcc

SEQ ID NO: 40 - protein, 415 aa, synthetic construct
MGSSHHHHHHSSGLVPRGSHMDNGDRLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSRGSAASMSSGNILWGSQNPIVFKNSFGVSNADTGSQDDLSQQNPFAEGYGVLLILLMVIQAIANNKFIEVQKNAERARNTQEKSNEMDEVIAKAAKGDAKTKEEVPEDVIKYMRDNGILIDGMTIDDYMAKYGDHGKLDKGGLQAIKAALDNDANRNTDLMSQGQITIQKMSQELNAVLTQLTGLISKWGEISSMIAQKTYS

SEQ ID NO: 41 - protein, 887 aa, synthetic construct
GSAASMNITTLTNSISTSSFSPNNTNGSSTETVNSDIKTTTSSHPSSLTMLNDTLHNIRTTNQALKKELSQKTLRNEYPINKDARELLHSAPKEAELDGDQMISHRELWAKIANSINDINEQYLKVYEHAVSSYTQMYQDFSAVLSSLAGWISPGGNDGNSVKLQVNSLKKALEELKEKYKDKPLYPANNTVSQEQANKWLTELGGTIGKVSQKNGGYVVSINMTPIDNMLKSLDNLGGNGEVVLDNAKYQAWNGFSAEDETMKNNLQTLVQKYSNANSIFDNLVKVLSSTISSCTDTDKLFLHFLEMHNVSTTTTGFPLAKILTSTELGDNTIQAANDAANKLFSLTIADLTANQNINTTNAHSTSNILIPELKAPKSLNASSQLTLLIGNLIQILGEKSLTALTNKITAWKSQQQARQQKNLEFSDKINTLLSETEGLTRDYEKQINKLKNADSKIKDLENKINQIQTRLSNLDPESPEKKKLSREEIQLTIKKDAAVKDRTLIEQKTLSIHSKLTDKSMQLEKEIDSFSAFSNTASAEQLSTQQKSLTGLASVTQLMATFIQLVGKNNEESLKNDLALFQSLQESRKTEMERKSDEYAAEVRKAEELNRVMGCVGKILGALLTIVSVVAAAFSGGASLALAAVGLALMVTDAIVQAATGNSFMEQALNPIMKAVIEPLIKLLSDAFTKMLEGLGVDSKKAKMIGSILGAIAGALVLVAAVVLVATVGKQAAAKLAENIGKIIGKTLTDLIPKFLKNFSSQLDDLITNAVARLNKFLGAAGDEVISKQIISTHLNQAVLLGESVNSATQAGGSVASAVFQNSASTNLADLTLSKYQVEQLSKYISEAIEKFGQLQEVIADLLASMSNSQANRTDVAKAILQQTTA

SEQ ID NO: 42 - DNA, 777 bp, synthetic construct
  source 1..777: mol_type = other DNA
atggtaaagataatatttgtgttttttattttcttatcatcattttcatatgcaaatgatgataagttatatcgggcagattctagacctcctgatgaaataaagcagtcaggtggtcttatgccaagaggacagagtgagtactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgatcagtaatacttgcgatgaaaaaacccaaagtctaggtgtaaaattccttgacgaataccaatctaaagttaaaagacaaatattttcaggctatcaatctgatattgatacacataatagaattaaggatgaattatga

SEQ ID NO: 43 - protein, 258 aa, synthetic construct
MVKIIFVFFIFLSSFSYANDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 44 - DNA, 375 bp, synthetic construct
  source 1..375: mol_type = other DNA
atgattaaattaaaatttggtgttttttttacagttttactatcttcagcatatgcacatggaacacctcaaaatattactgatttgtgtgcagaataccacaacacacaaatatatacgctaaatgataagatattttcgtatacagaatctctagctggaaaaagagagatggctatcattacttttaagaatggtgcaatttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 45 - protein, 124 aa, synthetic construct
MIKLKFGVFFTVLLSSAYAHGTPQNITDLCAEYHNTQIYTLNDKIFSYTESLAGKREMAIITFKNGAIFQVEVPGSQHIDSQKKAIERMKDTLRIAYLTEAKVEKLCVWNNKTPHAIAAISMAN

(19) United States
(12) Patent Application Publication
(10) Pub. No.: US 2024/0299522 A1
(43) Pub. Date: Sep. 12, 2024

(54) Methods and compositions related to the next generation vaccine

(71) Applicant: UNIVERSITY OF KANSAS, Lawrence, KS (US)

(72) Inventors: Wendy L. PICKING, Lawrence, KS (US); William D. PICKING, Lawrence, KS (US)

(21) Appl. No.: 18/543,534

(22) Filed: Dec. 18, 2023

Related U.S. Application Data
(60) Provisional application No. 63/433,285, filed on Dec. 16, 2022.

(51) Int. Cl. A61K 39/112; A61K 39/00; A61P 37/04; C07K 14/245; C07K 14/25; C07K 14/255

(52) U.S. Cl. CPC A61K 39/0283; A61K 39/0275; A61P 37/04; C07K 14/245; C07K 14/25; C07K 14/255; A61K 2039/55544; C07K 2319/00

(57) ABSTRACT

Disclosed are compositions comprising a Gram negative needle tip protein and a translocator protein and methods of their use.

### II. CROSS REFERENCE TO RELATED APPLICATIONS

This application claims the benefit of U.S. Provisional Application No. 63/433,285, filed on Dec. 16, 2023, which is incorporated herein by reference in its entirety.

### I. STATEMENT OF GOVERNMENT SUPPORT

This invention was made with government support under AI138970 awarded by National Institutes of Health. The government has certain rights in the invention.

### III. REFERENCE TO SEQUENCE LISTING

The sequence listing submitted on Dec. 18, 2023, as an .XML file entitled “10776-026US1.XML” created on Dec. 15, 2023, and having a file size of 65,536 bytes is hereby incorporated by reference pursuant to 37 C.F.R. § 1.52(e)(5).

### IV. BACKGROUND

The shigellae are intracellular bacteria that cause the intestinal disease shigellosis, which can result in severe diarrhea or dysentery. Shigellosis is a significant public health problem, with children especially vulnerable to increased morbidity and mortality. While most cases of shigellosis occur in developing nations, the shigellae also cause diarrhea among travelers and military personnel from developed countries.

Although there has been a reduction in the incidence of shigellosis globally due to improved sanitation, the rise of antimicrobial resistance in Shigella spp. warrants the development of a vaccine against this pathogen. At present, there is no licensed Shigella spp. vaccine, however, some killed cell and live-attenuated vaccines are currently in clinical trials. Unfortunately, the lack of cross-protection, strict storage conditions, and potential risks of contamination limit their use in developing countries. What are needed are new vaccines for Shigella.

### V. SUMMARY

Disclosed are methods and compositions related to polypeptides comprising a fusion of the needle tip protein and translocator protein of a type III secretion apparatus (T3SA) from a type III secretion system (T3SS) of a Gram negative bacteria.

Disclosed herein are fusion polypeptides comprising a fusion of a needle tip protein (such as, for example IpaD, SipD, or SseB) or an antigenic fragment thereof and a translocator protein (such as, for example, IpaB, SipB, or SseC) or an antigenic fragment thereof from a Type III secretion system (T3SS) of a Gram negative bacteria (such as, for example, Salmonella spp or Shigella spp). In one aspect, the fusion polypeptide comprises a Shigella spp fusion of the IpaD needle tip protein and the IpaB translocator protein and comprises the amino acid sequence as set forth in SEQ ID NO: 41 (i.e., DBF). In one aspect, the fusion polypeptide comprises a Salmonella spp fusion of the SipD needle tip protein and the SipB translocator protein and comprises the amino acid sequence as set forth in SEQ ID NO: 26 (i.e., S1). In one aspect, the fusion polypeptide comprises a Salmonella spp fusion of the SseB needle tip protein and the SseC translocator protein and comprises the amino acid sequence as set forth in SEQ ID NO: 36 (i.e., S2).

In one aspect, disclosed herein are fusion polypeptides, wherein the fusion polypeptide is arranged such that the needle tip protein is 5′ of the translocator protein.

Also disclosed herein are fusion polypeptides of any preceding aspect, wherein the fusion further comprises an adjuvant such as, for example, Cholera Toxin or antigenic fragment thereof (such as, for example, CTA1) or double mutant labile toxin (dmLT) or an antigenic fragment thereof labile toxin (such as, for example, LTA1) from Enterotoxigenic Escherichia coli. In some aspect, the dmLT or fragment thereof can also be fused to the needle tip protein-translocator protein fusion at the 5′ end. For example, the fusion polypeptide can comprise a DBF fusion protein comprising LTA-1 (i.e., L-DBF as set forth in SEQ ID NO: 16), a S1 fusion protein comprising LTA-1 (i.e., L-DBF as set forth in SEQ ID NO: 28), or a S2 fusion protein comprising LTA-1 (i.e., L-DBF as set forth in SEQ ID NO: 38).

In one aspect, disclosed herein are fusion polypeptides of any preceding aspect, wherein the fusion polypeptide further comprises pertussis toxoid (PTd).

Also disclosed herein are compositions (such as, for example vaccines) comprising the fusion polypeptides of any preceding aspect. For example, disclosed herein are compositions comprising a T3SA needle tip protein (such as, for example, IpaD, SipD, or SseB) or an antigenic fragment thereof from a Gram negative bacteria (such as, for example, Shigella spp, or Salmonella spp.) and a T3SA first translocator protein (such as, for example, IpaB, SipB, or SseC) or an antigenic fragment thereof from a Gram negative bacteria. In one aspect, the composition can comprise the needle tip protein or fragment thereof and the translocator protein or fragment thereof as separate components or as a fusion polypeptide (e.g., DBF (SEQ ID NO: 41), S1 (SEQ ID NO: 26), or S2 (SEQ ID NO: 36)). Also disclosed herein are compositions of any preceding aspect, wherein the composition comprises an adjuvant (such as, for example, dmLT, LTA1, cholera toxin, or CTA1) and/or bacterial toxin protein such as a pertussis toxoid (PTd). In some aspects, the composition can be emulsified using a MedImmune Emulsion (ME) or an emulsion of Squalene (8% w/v) and polysorbate 80 (2% w/v weight) (NE). The fusion polypeptide (such as, for example, DBF, S1, S2, L-DBF, L-S1, L-S2) can be at a final concentration of between 0.5 mg/mL and 1.0 mL (including, but not limited to 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0 mg/mL. In some aspects the composition can further comprise Bacterial Enzymatic Combinatorial Chemistry candidate 438 (BECC438).

In one aspect, disclosed herein are vaccines comprising the fusion polypeptides or compositions of any preceding aspect. In some embodiments, the vaccine can further comprise an acellular gram negative vaccine or active components thereof. In one aspect, the vaccine can comprise pertussis toxoid (PTd).

Also disclosed herein are methods of treating, inhibiting, or preventing an infection of a Gram negative bacteria (such as, for example, Shigella spp, or Salmonella spp) in a subject comprising administering to the subject the fusion polypeptide, composition, or vaccine of any preceding aspect.

In one aspect, disclosed herein are methods of treating, inhibiting, or preventing an infection of a Gram negative bacteria of any preceding aspect, wherein the method further inhibits or prevents colony formation of the bacteria and/or transmission of the bacteria to another subject. In one aspect, the use of the fusion polypeptide in the methods of treatment is not dependent of the serotype of any prior infection.

Also disclosed herein are methods of eliciting an immune response in a subject to a Gram negative bacteria (such as, for example, Shigella spp, or Salmonella spp) comprising administering to the subject the fusion polypeptide, composition, or vaccine of any preceding aspect. For example, disclosed herein are methods of eliciting an immune response against at least one Gram negative bacteria serovar in a subject in need thereof, comprising administering to the subject a composition comprising at least one needle tip protein or an antigenic fragment thereof and/or at least one translocator protein or an antigenic fragment thereof; wherein said composition is administered in an amount sufficient to elicit an immune response to said at least one Gram negative bacteria serovar in said subject.

In one aspect, disclosed herein are methods of eliciting an immune response in a subject to a Gram negative bacteria of any preceding aspect, wherein the immune response provides sterilizing immunity.

### VI. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows protective efficacy of L-DBF against lethal Shigella challenge in mice. Balb/C mice (n=10) were vaccinated intranasally (IN) on days 0, 14 and 28 with PBS, 20 μg DBF+2.5 μg dmLT and 25 μg L-DBF. On day 56, the mice were challenged IN with 6×10⁶ CFU/mouse of S. flexneri 2a 2457T. Significance was calculated by comparing vaccinated groups to the PBS group with Log-rank (Mantel-Cox) tests for survival (20 μg DBF+2.5 μg dmLT vs. PBS: p<0.001; 25 μg L-DBF vs. PBS: p<0.001).

FIGS. 2A and 2B show the kinetics of serum IgG and fecal IgA responses. Mice were vaccinated IN three times (Day 0, 14 and 28). Blood and fecal samples were collected and measured titers for anti-IpaB (2A) or anti-IpaD (2B) IgG (solid lines) and IgA (dotted lines) by ELISA. The individual titers are represented as EU ml−1. Each point represents the mean and error bars represent SD of each group (n=14/group).

FIG. 3 shows the frequency of IL-17A and IFN-γ secreting cells after antigen-specific stimulation. Single cell spleen (left) and lung (right) suspensions were used to assess antigen specific IL-17A and IFN-γ secreting cells. Cells were incubated with 10 μg IpaB or IpaD. IL-17A and IFN-γ secreting cells were enumerated by ELISpot and are presented here as spot forming cells/10⁶ cells. The original data were rescaled and normalized using the equation Ynormal=(Yorigin−Ymin)/(Ymax−Ymin). The data are boxed from minimum to maximum for each group after normalization. Significance was calculated by comparing groups that were unvaccinated (PBS) and mice vaccinated with antigens using Dunnett's test. *p<0.05; **p<0.01; ***p<0.001.

FIG. 4 shows the levels of T cell related cytokines secreted from lung cells after stimulation with IpaB or IpaD. The single lung cell suspensions were used to assess antigen specific IFN-γ, IL-17A, IL-6 and TNF-α secretion from lung cell suspensions. Cells were incubated with 10 μg IpaB (left) or IpaD (right). Cytokine levels were determined by Meso Scale Discovery analysis as per manufacturer's specifications and are presented here as pg/ml/10⁶ cells. The original data were rescaled and normalized for comparative purposes using the equation Ynormal=(Yorigin−Ymin)/(Ymax−Ymin). The data are boxed as minimum to maximum in each group after normalization. Significance was calculated by comparing groups that were unvaccinated (PBS) and mice vaccinated with antigens using Dunnett's test. **p<0.01; ***p<0.001.

FIG. 5 shows The LTA1 moiety of L-DBF was shown to transfer biotin-ADPr from biotin-NAD+ to itself and ARF4. The biotin-ADPr staining was visualized using western blot analysis followed by probing with Streptavidin-IR800

FIG. 6 shows Antigen-specific IgG and IgA responses for mice vaccinated that were subsequently challenged with heterologous Shigella spp. Mice were vaccinated IN with PBS or 25 μg L-DBF three times (Day 0, 14 and 28). Blood and fecal samples were collected and serum titers for serum IgG (solid symbols) and fecal IgA (open symbols) specific for IpaB or IpaD were measured by ELISA. The group titers are represented as EU ml−1. Each point represents the mean and error bars represent SD of each group (n=10/group).

FIGS. 7A, 7B, 7C, 7D, 7E, 7F, 7G, 7H, 7I, and 7J show cross-protection of L-DBF against the challenges from five different subtype Shigella strains in mice. Mice (n=10) vaccinated with PBS and 25 μg of L-DBF intranasally on days 0, 14, 28. On day 56, the mice were then challenged IN with S. flexneri 2a (6×10⁶ CFU per mouse), S. flexneri 3a (1×10⁶ CFU per mouse), S. flexneri 6 (1×10⁶ CFU per mouse), S. flexneri 1b (4×10⁶ CFU per mouse), and S. sonnei 53G (1×10⁶ CFU per mouse) separately, and monitored for weight loss every 24 h (Panels 7B, 7D, 7F, 7J and 7H). The group titers are represented as EU ml−1. Significance was calculated by comparing vaccinated groups to the PBS group with Log-rank (Mantel-Cox) tests in survival tests (all p<0.001.

FIGS. 8A, 8B, 8C, and 8D show dose escalation of L-DBF against the challenges from Shigella flexneri 2a strains in mice. Mice (n=10) vaccinated with PBS and 1 μg, 10 μg, 25 μg of L-DBF (8A) or PBS and 15 μg, 25 μg of L-DBF (8C) intranasally on days 0, 14, 28. On day 56, the mice were then challenged IN with 6×10⁶ CFU per mouse of S. flexneri 2a (8A) and 1.5×10⁶ CFU per mouse of S. flexneri 2a (8C), separately, and monitored the weight loss every 24 h (Panel 8B & 8D). The group titers are represented as EU ml−1. Significance was calculated by comparing vaccinated groups to the PBS group with Log-rank (Mantel-Cox) tests in survival tests. *p<0.001.

FIGS. 9A and 9B show the single cell spleen (left) and lung (right) suspensions were used to quantify antigen specific IL-17A and IFN-γ secreting cells. Cells were incubated with 10 μg IpaB or IpaD. IL-17A and IFN-γ secreting cells were enumerated by ELISpot and are presented here as spot forming cells/10⁶ cells. Significance was calculated by comparing groups that were unvaccinated (PBS) and mice vaccinated with antigens using Dunnett's test. *p<0.001.

FIGS. 10A and 10B show the single lung cell suspensions were used to assess antigen specific IFN-γ, IL-17A, IL-6 and TNF-α secretion from lung cell suspensions. Cells were incubated with 10 μg IpaB (left) or IpaD (right). Cytokine levels were determined by Meso Scale Discovery analysis as per manufacturer's specifications and are presented here as pg/ml/10⁶ cells. Significance was calculated by comparing groups that were unvaccinated (PBS) and mice vaccinated with antigens using Dunnett's test. *p<0.001.

FIGS. 11A and 11B show the single splenocyte suspensions were used to assess antigen specific IFN-γ, IL-17A, IL-6 and TNF-α secretion from splenocyte suspensions. Splenocytes were incubated with 10 μg IpaB (left) or IpaD (right). Cytokine levels were determined by Meso Scale Discovery analysis as per manufacturer's specifications and are presented here as pg/ml/10⁶ cells. Significance was calculated by comparing groups that were unvaccinated (PBS) and mice vaccinated with antigens using Dunnett's test. *p<0.001

FIGS. 12A, 12B, 12C, 12D, 12E, and 12F show the kinetics of serum IgG and fecal IgA titers from mice exposed twice to S. flexneri 2a. Half of the mice were infected (treated or T or untreated NT) with a sublethal dose of S. flexneri 2a on days −56 and −28. Half of each group of mice were then vaccinated IN three times (days 0, 14, and 28) (vaccinated or V or not vaccinated NV). Blood and fecal samples were collected and measured titers for anti-LPS (Panel 12A & 12D) or anti-IpaB (Panel 12B & 12E) or anti-IpaD (Panel 12C & 12F) IgG and IgA by ELISA. More than 40% of serum samples from the T-NV group did not have detectable titers of anti-IpaD during all the timepoints. More than 50% of the serum samples from the NT-V group did not have detectable titers of anti-LPS during all the timepoints. The individual titers are represented as EU ml−1. Each point represents the mean of each group (n=10/group).

FIGS. 13A, 13B, and 13C show serum bacteriocidal activity (SBA) in serum from mouse groups after two S. flexneri pre-exposures. The killing activity (%) in serially diluted serum collected on day 28 after the first pre-infection (pre-1) or on day 14 after the second pre-infection (pre-2) is shown (Panel 13A). The killing activity (%) in serially diluted serum collected on day 42 (Panel 13B) or day 55 (Panel 13C) from NT-V, T-NV, or T-V groups is shown. A dashed horizontal line indicates 50% killing. CFU counts obtained from wells incubating with serum from NT-NV groups were used as 0% killing (baseline). Killing activity (%)=(Spots in NTNV well−spots in test well)/Spots in NT-NV well]. Duplicate experiments with triplicate wells for each test point is presented. Mann-Whitney test was used for analysis. *p<0.05; **p<0.01; ***p<0.001

FIGS. 14A and 14B show SBA in serum from mouse groups after two S. flexneri pre-exposures in competition with exogenously added S. flexneri 2a LPS. The killing activity (%) for serum collected on day 42 (Panel 14A) or day 55 (Panel 14B) is shown for the NT-V group (1:64 dilution), T-NV group (1:512 dilution) or T-V group (1:512 dilution) in competition with added LPS that is serially diluted. A dashed horizontal line indicates the 50% killing threshold. The viable CFU obtained for wells incubated with serum from the NT-NV groups were used as the 0% killing baseline. Killing %=(Spots in NT-NV well−spots in test well)/Spots in NT-NV well]. Experiments were performed in duplicate with triplicate wells for each test point. The Mann-Whitney test was used for statistical comparisons. *p<0.05; **p<0.01; ***p<0.001

FIGS. 15A, 15B, 15C, and 15D show SBA for serum from mouse groups after two S. flexneri pre-exposures in competition with exogenously added IpaB or IpaD. The killing activity (%) in serum collected on day 42 (Panel 15A) or day 55 (Panel 15C) from NT-V groups (1:8), T-NV groups (1:512) or from T-V groups (1:512) in competition with serially diluted IpaB is shown. The killing activity (%) in serum collected on day 42 (Panel 15B) or day 55 (Panel 15D) from NT-V groups (1:8), T-NV groups (1:512) or from T-V groups (1:512) in competition with serially diluted IpaD is shown. The dashed horizontal line indicates the 50% killing threshold. CFU counts obtained with wells incubating with serum from the NT-NV groups were used as the 0% killing baseline. Killing %=(Spots in NT-NV well−spots in test well)/Spots in NT-NV well]. Experiments were performed in duplicate with triplicate wells for each test point. The Mann-Whitney test was used for analysis. *p<0.05; **p<0.01; ***p<0.001

FIG. 16 shows the frequency of IL-17 and IFN-γ secreting cells following antigen-specific stimulation. Lung single cell suspensions were used to assess antigen-specific IFN-γ and IL-17 secreting cells using ELISpot analysis. Cells from each animal group (using the same designations as in the previous figures) were incubated with 10 μg IpaB (left side) or IpaD (right side). IFN-γ and IL-17 secreting cells were then enumerated by ELISpot as described in Methods and are presented here as spot-forming cells/10⁶ cells. The data are plotted as means±SD for individual mice in each group. Significance was calculated by comparing groups that were unvaccinated (PBS) and mice vaccinated with antigens using two-way ANOVA. *p<0.05; **p<0.01; ***p<0.001.

FIG. 17 shows quantification of T cell-related cytokines secreted from lung cells after stimulation with IpaB or IpaD. The lung single cell suspensions described in FIG. 17 were also used to assess the amount of antigen-specific IFN-γ, IL-17 and IL-6 secreted from these cells. Cells were incubated with 10 μg IpaB (left) or IpaD (right). Cytokine levels were then measured by Meso Scale Discovery analysis as per the manufacturer's specifications and are presented here as pg/ml/10⁶ cells. The data are plotted as the mean±SD for the individual mice in each group. Significance was calculated by comparing groups that were unvaccinated (PBS) and mice vaccinated with antigens using two-way ANOVA. *p<0.05; **p<0.01; ***p<0.001.

FIGS. 18A, 18B, and 18C show the kinetics of serum IgG and fecal IgA titers from mice after single pre-exposure. Mice were vaccinated intranasally (IN) three times (Day 0, 14, and 28). Blood and fecal samples were collected. Kinetics of resulting titers were measured for anti-IpaB (panel 18A), anti-IpaD (panel 18B) or anti-LPS (panel 18C) IgG (solid) and IgA (dotted) by ELISA. The individual titers are represented as EU ml−1. Each point represents the mean of each group (n=10/group). NT-NV: no pre-exposure and PBS vaccination; NT-V: no pre-exposure and L-DBF vaccination; T-NV: pre-exposure and vaccinated with PBS; T-V: pre-exposure and L-DBF vaccinated. The log=2 (black dotted line) is the cutting level of the background.

FIG. 19 shows the protective efficacy of L-DBF against a Shigella lethal challenge in mice. Balb/C mice (n=10) were pre-exposed to a single sublethal dose of S. flexneri 2a on day-60 and then vaccinated IN on days 0, 14 and 28 with 25 μg L-DBF. On day 56, the mice were challenged IN with S. flexneri 2a, S. flexneri 1b or S. sonnei.

FIGS. 20A and 20B show the kinetics of serum IgM responses from mice with two sublethal doses of S. flexneri 2a on days −56 and −28. Mice were then vaccinated IN three times (days 0, 14, and 28). Blood and fecal samples were collected, and the titers determined for anti-IpaB (solid) or IpaD (dotted) (Panel 20A) or anti-LPS (Panel 20B) IgM by ELISA. The individual titers are represented as EU ml−1. Each point represents the mean of each group (n=10/group).

FIG. 21 shows the protective efficacy of L-DBF against the Shigella lethal challenge in mice. Balb/C mice (n=10) were pre-exposed to two sublethal doses of S. flexneri 2a on days −56 and −28 and then vaccinated intranasally (IN) on days 0, 14 and 28 with 25 μg L-DBF. On day 56, the mice were challenged IN with S. flexneri 2a, S. flexneri 1b or S. sonnei.

FIGS. 22A, 22B, 22C, and 22D show serum bacteriocidal activity (SBA) activity for serum from mice after a single S. flexneri 2a pre-exposure. The killing activity (%) in serially diluted pooled serum from NT-NV, T-NV or the NT-V group is shown (Panel 22A). The killing activity (%) in serum from the T-NV group (1:512 dilution) or from NT-V groups (1:64) in competition with serially diluted LPS is shown in Panel 22B. The killing activity (%) in serum from the T-NV group (1:512 dilution) or from the NT-V group (1:8 dilution) in competition with serially diluted IpaB (Panel 22C) or IpaD (Panel 22D) is also shown. Dashed horizontal lines indicate the 50% killing point. CFU counts obtained from wells incubated with serum from the NT-NV group was used as 0% killing (baseline control). Killing activity (%)=(Spots in NT-NV well−spots in test well)/Spots in NT-NV well]. Duplicate experiments with triplicate wells for each test point. Mann-Whitney test was used for analysis. *p<0.001.

FIGS. 23A, 23B, and 23C shows competitive SBA of the samples from mice receiving two sublethal pre-infections with exogenously added S. flexneri 2a LPS, IpaB or IpaD. The killing activity (y axis) after incubating with pre-1 (serum collected on day 28 after first pre-infection) or pre-2 (serum collected on day 14 after second pre-infection) in the presence of serially diluted LPS (Panel 23A) or IpaB (Panel 23B) or IpaD (Panels 23C) are shown. CFU counts obtained with wells incubated with serum from the NT-NV group was used as 0% killing (baseline). Killing %=(Spots in NT-NV well−spots in test well)/Spots in NT-NV well]. Duplicate experiments with triplicate wells for each test point. Mann-Whitney test was used for analysis. *p<0.001.

FIG. 24 shows the frequency of IL-17 and IFN-γ secreting cells after antigen-specific stimulation. The splenocytes were used to enumerate the antigen-specific IFN-γ and IL-17 secreting cells. Cells were incubated with 10 μg IpaB (left) or IpaD (right). IFN-γ and IL-17 secreting cells were enumerated by ELISpot analysis and are presented here as spot-forming cells/10⁶ cells. The data are plotted as means±SD for individual mice in each group. Significance was calculated by comparing groups that were unvaccinated (PBS) and mice vaccinated with antigens using two-way ANOVA. *p<0.001

FIG. 25 shows the concentrations of T cell-related cytokines secreted from splenocytes was determined after stimulation with IpaB or IpaD. Single spleen cell suspensions were used to assess antigen-specific IFN-γ, IL-17 and IL-6 secretion. Cells were incubated with 10 μg IpaB (left) or IpaD (right). Cytokine concentrations were determined by Meso Scale Discovery analysis as per the manufacturer's specifications and are presented here as pg/ml/10⁶ cells. The data are plotted as means±SD for individual mice in each group. Significance was calculated by comparing groups that were unvaccinated (PBS) and mice vaccinated with antigens using two-way ANOVA. *p<0.001.

FIGS. 26A and 26B show IL-17A and IFN-γ secretion from isolated lung cells from mice vaccinated with DBF+BECC438 with ME or NE. All samples were collected from mice three days before a parallel group was challenged. FIG. 26A shows single lung cell suspensions were prepared and used to assess antigen specific IL-17 and IFN-γ secreting cells after antigen stimulation. Cells were stimulated with 10 μg IpaB (left) or IpaD (right). IL-17 and IFN-γ secreting cells were enumerated by ELISpot and are presented here as spot forming cells/10⁶ total cells. FIG. 26B shows single lung cell suspensions were stimulated with 10 μg IpaB (left) or IpaD (right). Cytokine levels in the cell supernatants were then determined by Meso Scale Discovery analysis as per manufacturer's specifications and are presented here as pg/ml/10⁶ cells. Data were plotted as actual values from individuals±SD (n=4) in each group. Significance was calculated by comparing groups that were unvaccinated (PBS) and mice vaccinated with antigens using the Welch t-test. *p<0.05; **p<0.01; ***p<0.001

FIGS. 27A and 27B show IL-17 and IFN-γ secretion from isolated lung cells from mice vaccinated with DBF+BECC438 alone, with ME or with Chi-C48/80. All samples were collected three days prior to a parallel group being challenged. FIG. 27A shows lung cell suspensions were prepared, and the antigen-specific IL-17 and IFN-γ secreting cells were assessed. Cells were stimulated with 10 μg IpaB (left) or IpaD (right). The quantification of IL-17 and IFN-γ secreting cells was determined using ELISpot, and the results are presented as spot forming cells/10⁶ total cells. FIG. 27B shows following the preparation of single cell suspensions, cells were stimulated with 10 μg IpaB (left) or IpaD (right). The levels of cytokines in the cell supernatants were measured using Meso Scale Discovery analysis, according to the manufacturer's specifications. The results are presented as pg/ml/10⁶ cells. Data were plotted as actual values from individuals±SD (n=4) in each group. Statistical significance was determined by performing the Welch t-test, comparing the unvaccinated group (PBS) with the mice vaccinated with antigens. *p<0.05; **p<0.01; ***p<0.001 indicate the level of significance.

FIGS. 28A and 28B show IL-17 and IFN-γ secretion from isolated lung cells from mice vaccinated with a dose escalation of L-DBF in a formulation with BECC438+ME. All samples were collected three days before a parallel group was challenged. FIG. 28A shows that to assess antigen-specific IL-17 and IFN-γ secreting cells, single lung cell suspensions were prepared. Cells were stimulated with 10 μg IpaB (left) or IpaD (right). IL-17 and IFN-γ secreting cells was performed by ELISpot and are presented here as spot forming cells/10⁶ cells. FIG. 28B shows single cell suspensions were then stimulated with 10 μg IpaB (left) or IpaD (right) and the levels of cytokines in the cell supernatants were determined by Meso Scale Discovery analysis following the manufacturer's specifications, and are presented here as pg/ml/10⁶ cells. The data are displayed as the actual values obtained from individual samples, accompanied by the mean±SD (n=4) for each group. Significance was calculated by comparing groups that were unvaccinated (PBS) and mice vaccinated with antigens using the Welch t-test. *p<0.05; **p<0.01; ***p<0.001

FIGS. 29A and 29B show IL-17 and IFN-γ secretion from isolated lung cells from mice vaccinated with BECC438+L-DBF+ME using distinct booster regimens. All samples were collected three days before a parallel group of mice were challenged. FIG. 29A shows the enumeration of IL-17 and IFN-γ secreting cells specific to the antigen was conducted using ELISpot on single lung cell suspensions. Stimulation with 10 μg of IpaB (left) or IpaD (right) was employed for this purpose. The resulting spot forming cells/10⁶ total cells were recorded and analyzed. FIG. 29B shows the measurement of cytokine levels in the cell supernatants was performed using Meso Scale Discovery analysis, following the manufacturer's specifications. Single cell suspensions were stimulated with 10 μg of IpaB (left) or IpaD (right), and the cytokine levels were quantified as pg/ml/10⁶ cells. Data were plotted as actual values from individuals±SD (n=4) in each group. The significance of the results was assessed using the Welch t-test, comparing the unvaccinated group (PBS) with the group of mice vaccinated with antigens. Significance levels are denoted as *p<0.05, **p<0.01, and ***p<0.001.

FIG. 30 shows the correlation between protection and the secretion of IFN-γ and IL-17 after stimulating with IpaB. The original data (cytokine levels from FIGS. 1 to 4) were rescaled (normalized) into the [0, 1] range using Yₙₒᵣₘₐₗ=(Y_{origin}−Yₘᵢₙ)/(Yₘₐₓ−Yₘᵢₙ). The normalized data were then used to establish a model comparing IFN-γ (X) and IL-17A (Y) with protection (left; color map). A >60% vaccine efficacy was used as a targeted cut-off value (right).

FIG. 31 shows the correlation between protection and the secretion of IFN-γ and IL-17 after stimulating with IpaD. The initial cytokine data (from FIGS. 1 to 4) underwent rescaling (normalization) to fit within the [0, 1] range using the formula Yₙₒᵣₘₐₗ=(Y_{origin}−Yₘᵢₙ)/(Yₘₐₓ−Yₘᵢₙ). The normalized data was subsequently utilized to construct a model, correlating IFN-γ (X) and IL-17A (Y) with the degree of protection (left; color map). A threshold of >60% vaccine efficacy was employed as the designated cut-off value (right).

FIG. 32 shows the correlation between protection and the sum of secreted IFN-γ or IL-17 after stimulating with IpaB or IpaD. The raw cytokine data (as depicted in FIGS. 1 to 4) underwent a transformation process to fit within the normalized range of [0, 1]. The normalized data were used to establish a model that involved the sum (ranging from 0 to 2) of IFN-γ stimulating with IpaB or IpaD (X; IFN-γ sum=IFN-γ of IpaB+IFN-γ of IpaD) and the sum (ranging from 0 to 2) of IL-17A stimulating with IpaB or IpaD (Y; IL-17A sum=IL-17A of IpaB+IL-17A of IpaD) with protection (left; color map). To determine a threshold for significant vaccine efficacy, a cut-off value of >60% was selected (right).

FIGS. 33A and 33B show IL-17A and IFN-γ secretion from lung cells prepared from mice vaccinated intramuscularly (IM) with 5 μg (33A) or 50 μg (33B) BECC438b+DBF on Day 3 after challenge. All samples were collected on day 3 after the challenge. Single-cell lung suspensions were incubated with 10 μg IpaB and IpaD. Cytokine levels were determined by Meso Scale Discovery analysis as per the manufacturer's specifications and are presented here as pg/ml/10⁶ cells. Secretion of IFN-γ and IL-17A was noted as a response of either IpaB or IpaD stimulation. Data were plotted as actual values from individuals±SD (n=4) in each group. Significance was calculated by comparing groups that were unvaccinated (PBS) and mice vaccinated with antigens using a Welch t-test. *p<0.001.

FIGS. 34A and 34B show antigen-specific IgG and IgA responses. Mice were vaccinated intranasally (IN) with PBS, 20 μg DBF+2.5 μg dmLT, 50 μg, 25 μg or 5 μg BECC438b+20 μg DBF three times (Day 0, 14 and 28). Blood and fecal samples were collected and serum titers for serum IgG (filled symbols with solid lines) and fecal IgA (open symbols with dashed lines) specific for IpaB (34A) or IpaD (34B) were measured by ELISA. The individual titers are represented as EU ml−1. Each point represents the mean and error bars represent SD of each group (n=10/group).

FIGS. 35A, 35B, and 35C show The weight loss of mice following Shigella challenge. Mice vaccinated with different formulations via IM (Panel 35A & 35B) or IN (Panel 35C) routes were then challenged IN with 6×10⁶ CFU per mouse of S. flexneri 2a and their weights monitored every 24 h for 14 days.

FIGS. 36A and 36B show all samples were collected on day 14 after the challenge. The single-cell splenocyte suspensions were used to assess antigen-specific IFN-γ and IL-17A (36A), IL-6 and TNF-α (36B) secretion from spleen cell suspensions. Cells were incubated with 10 μg IpaB or IpaD. Cytokine levels were determined by Meso Scale Discovery analysis as per the manufacturer's specifications and are presented here as pg/ml/10 6 cells. Since all mice in the PBS group died after days 5 post-challenge, significance in this study was calculated by comparing groups that were vaccinated with 20 μg DBF+2.5 μg dmLT and mice vaccinated with BECC438b formulations using Welch t-test. +p<0.001.

FIGS. 37A and 37B show antigen-specific IgG and IgA responses were assessed in mice following IN administration with different formulations. The vaccination groups included PBS, 20 μg DBF+2.5 μg dmLT, 50 μg, 5 μg, or 0.5 μg BECC438b+20 μg DBF+ME or NE. Vaccinations were administered three times on Day 0, 14, and 28. Blood and fecal samples were collected, and serum titers for IgG (represented by filled symbols with solid lines) and fecal IgA (represented by open symbols with dashed lines) specific for IpaB (left) or IpaD (right) were determined using ELISA. The individual titers are expressed as EU ml−1. Each data point represents the mean, and the error bars indicate the SD of each group (n=10/group).

FIGS. 38A and 38B show antigen-specific IgG and IgA responses were evaluated in mice following IN vaccination with PBS, 25 μg L-DBF, 10 μg BECC438b+20 μg DBF with or without ME or with Chi-C48/80 IN three times (Day 0, 14 and 28). Blood and fecal samples were collected, and ELISA was performed to measure serum titers of IgG (represented by filled symbols with solid lines) and fecal IgA (represented by open symbols with dashed lines) specific for IpaB (left) or IpaD (right). The individual titers are represented as EU ml−1. Each point represents the mean and error bars represent SD of each group (n=10/group).

FIGS. 39A and 39B show antigen-specific IgG and IgA responses. The experimental groups of mice received IN vaccinations including PBS, 25 μg L-DBF via IN administration, and 10 μg BECC438b combined with 15 μg, 10 μg, or 1 μg L-DBF with ME on three separate occasions (Day 0, 14, and 28). Blood and fecal samples were collected and serum titers for serum IgG (filled symbols with solid lines) and fecal IgA (open symbols with dashed lines) specific for IpaB (left) or IpaD (right) were measured by ELISA. The individual titers are represented as EU ml−1. Each point represents the mean and error bars represent SD of each group (n=10/group).

FIGS. 40A and 40B show antigen-specific IgG and IgA responses. A separate cohort of mice received either PBS or 25 μg L-DBF intranasally on three occasions (Day 0, 14, and 28). Another cohort of mice received a single prime vaccination of 1 μg BECC438b+0.5 μg L-DBF+ME via IN route. A second group of mice received a prime vaccination followed by one booster dose (2 doses or 2D), and a third group received a prime vaccination followed by two booster doses (3 doses or 3D). Blood and fecal samples were collected, and serum titers of IgG (represented by filled symbols with solid lines) and fecal IgA (represented by open symbols with dashed lines) specific to IpaB (left) or IpaD (right) were measured using ELISA. The individual titers are expressed as EU ml−1. Each data point represents the mean value, and the error bars represent the standard deviation of each group (n=10/group).

FIG. 41 shows the protective efficacy of LTA1-DBF (also referred to herein as L-DBF) vs DBF+dmLT. Mice were vaccinated intramuscularly on days 0, 14 and 28 with the indicated μg of DBF+0.1 μg dmLT or DBF equivalent of LTA1-DBF. The positive control was DBF+dmLT delivered intranasally. On day 56 the mice were challenged with Shigella flexneri. FIG. 14 indicates the percent survival of mice post infection with Shigella flexneri.

FIGS. 42A, 42B, and 42C show the kinetics of IgG response. Mice from FIG. 14 were bled prior to vaccination and on day 42. Sera were assessed for anti-IpaD, -IpaB and -dmLT IgG, and the data is shown in (42A) IpaD, (42B) IpaB, and (42C) dmLT. Differences in the IgG levels in mice vaccinated with dmLT vs. LTA1 are attributed to the recognition of the entire dmLT on the well.

FIGS. 43A, 43B, and 43C show the stimulation of antibody secreting cells from bone marrow. Bone marrow was collected on day 56. Single cell suspensions from 5 mice per group were stimulated in vitro IpaD, IpaB or dmLT. IgG (black) and IgA (white) ASC were measured by ELISpot, and the data is shown in (43A) IpaD, (43B) IpaB, and (43C) dmLT. Bars represent mean ASC per 10⁶ cells+SD from replicate wells.

FIGS. 44A, 44B, and 44C show the stimulation of antibody secreting cells from spleen. Spleens were collected on day 56. Single cell suspensions from 5 mice per group were stimulated in vitro IpaD, IpaB or dmLT. IgG (black) and IgA (white) ASC were measured by ELISpot. Bars represent mean ASC per 10⁶ cells+SD from replicate wells.

FIGS. 45A, 45B, and 45C show the stimulation of antibody secreting cells from lungs. Lungs were collected on day 56. Single cell suspensions from 5 mice per group were stimulated in vitro IpaD, IpaB or dmLT. IgG (black) and IgA (white) ASC were measured by ELISpot, and the data is shown in (45A) IpaD, (45B) IpaB, and (45C) dmLT. Bars represent mean ASC per 10⁶ cells+SD from replicate wells.

FIG. 46 shows the ADPr activity of L-antigens. LTA1 was fused to DBF, 22BF or SseB. LTA1, however, must retain its ADP-ribosylation activity to maintain adjuvant activity. The ADPr of NAD+ was biotin conjugated and LTA1 transferred the biotin-ADPr moiety to ARF4. The biotin was then detected with Streptavidin-IR800. Lane 1: LTA1-DBF; 2: LTA1-22BF; 3: LTA1-SseB.

### VII. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

The term “inhibit” refers to a decrease in an activity, response, condition, disease, or other biological parameter. This can include, but is not limited to, the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The needle tip protein and/or translocator proteins or antigenic portions thereof disclosed herein are used to elicit an immune response in subjects to whom they are administered. By “elicit an immune response”, “induces or enhances an immune response”, or “stimulates an immune response” which are used interchangeably herein, is meant that the subject mounts one or both of an innate and/or an adaptive immune reaction against antigenic determinants of the proteins or antigenic portions thereof that are administered. Preferably a statistically measurable induction or increase in an immune response over a control sample to which the needle tip protein and/or translocator proteins or antigenic portions thereof disclosed herein has not been administered. Preferably the induction or enhancement of the immune response results in a prophylactic or therapeutic response in a subject. In particular, the adaptive immune reaction entails production of e.g. B and T cell lymphocytes and antibodies specific for binding and forming complexes with the antigenic determinants. In some embodiments, the proteins and/or antigenic fragments thereof elicit a protective immune response in the subject, i.e. administration of one or more of the proteins and/or antigenic portions thereof results in an immune response that is protective against later challenge by the disease causing organism itself, either preventing infection altogether, or lessening the impact of infection by decreasing disease symptoms that would otherwise occur, had the subject not been vaccinated as described herein.

“Vaccine” as used herein is a preparation that stimulates an immune response that produces immunity against particular antigens, e.g. Gram negative bacteria. Vaccines may be administered prophylactically (for example, to prevent or inhibit the establishment of an infection) or therapeutically to inhibit, reduce, or treat an established infection, or to ameliorate the effects or symptoms of an infection. Vaccines may contain, but are not limited to, live, attenuated infectious material such as viruses or bacteria, and dead or inactivated organisms or purified products derived therefrom. A vaccine can be administered by injection, orally, or by inhalation. Injections may be, but are not limited to, subcutaneous (sc), intramuscular (im), intraperitoneal (ip), intradermal (id) or intravenous (iv).

“Optional” or “optionally” means that the subsequently described event or circumstance may or may not occur, and that the description includes instances where said event or circumstance occurs and instances where it does not.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician or veterinarian.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a particular needle tip protein (such as, for example, IpaD, SipD, or SseB), translocator protein (such as, for example, IpaB, SipB, or SseC), or fusion polypeptide thereof (such as, for example, DBF, S1, or S2) is disclosed and discussed and a number of modifications that can be made to a number of molecules including the needle tip protein (such as, for example, IpaD, SipD, or SseB), translocator protein (such as, for example, IpaB, SipB, or SseC), or fusion polypeptide thereof (such as, for example, DBF, S1, or S2) are discussed, specifically contemplated is each and every combination and permutation of needle tip protein such as, for example, IpaD, SipD, or SseB), translocator protein (such as, for example, IpaB, SipB, or SseC), or fusion polypeptide thereof (such as, for example, DBF, S1, or S2) and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

To infect a host, Gram negative bacteria use an arsenal of well-characterized virulence factors. These factors include pertussis toxin (PT), adenylate cyclase toxin (ACT), the type III secretion system (T3SS), tracheal cytotoxin (TCT), dermonecrotic toxin (DNT), filamentous hemagglutinin (FHA), pertactin (PRN), and lipooligosaccharide (LOS). Current aP vaccines are comprised of PT, FHA, PRN, and the fimbrial proteins in varying proportions, but not necessarily all four proteins. Though the aP vaccine causes fewer adverse reactions than the wP vaccine, it is not as efficacious. This same situation exists for other pathogenic Gram negative bacteria. Accordingly, disclosed herein are fusion polypeptides from a Type III secretion system (T3SS) of a Gram negative bacteria (such as, for example, Shigella spp., Salmonella spp. (such as, for example, Salmonella enterica)) comprising a polypeptide of needle tip protein (such as, for example, IpaD, SipD, or SseB) or an antigenic fragment thereof and polypeptides of a translocator protein (such as, for example, IpaB, SipB, or SseC) or an antigenic fragment thereof. It is recognized and herein contemplated that the disclosed polypeptides can be separate components of a composition or more preferably a fusion construct. By a “fusion polypeptide” is meant a peptide, polypeptide, or protein that is translated from a single, contiguous nucleic acid molecule, and which comprises sequences from at least two different proteins or antigenic regions thereof. Typically, the individual sequences are joined via a linker or spacer sequence of e.g. from about 2 to about 20 amino acids, usually from about 2 to about 10 amino acids. The amino acids in linking sequences are typically uncharged and the linker sequence usually does not exhibit secondary or tertiary structure, but does allow the fused protein/peptide segments to adopt functional secondary, tertiary, etc. conformations. One such exemplary fusion polypeptide includes DBF2 (as set forth in SEQ ID NO: 41), S1 (as set forth in SEQ ID NO: 26 and encoded by the nucleic acid sequence as set forth in SEQ ID NO: 25), and S2 (as set forth in SEQ ID NO: 36 and encoded by the nucleic acid sequence as set forth in SEQ ID NO: 35). The chimera may be encoded by any suitable nucleic acid sequence.

Thus, in one aspect, disclosed herein are fusion polypeptides comprising a fusion of a needle tip protein (such as, for example, IpaD, SipD, or SseB) or an antigenic fragment thereof and a translocator protein (such as, for example, IpaB, SipB, or SseC) or an antigenic fragment thereof from a Type III secretion system (T3SS) of a Gram negative bacteria (such as, for example, Shigella spp., Salmonella spp.). For example, the fusion polypeptide can comprise a fusion of the Shigella spp. needle tip protein (IpaD) (as set forth in SEQ ID NO: 1 and encoded by the nucleic acid sequence as set forth in SEQ ID NO: 2) and first translocator protein (IpaB) (as set forth in SEQ ID NO: 3 and encoded by the nucleic acid sequence as set forth in SEQ ID NO: 4) or fragments thereof (the fusion referred to as DBF as set forth in SEQ ID NO: 41), Salmonella spp. (such as, for example, S. enterica) SPI-1 needle tip protein (SipD) (as set forth in SEQ ID NO: 22 and encoded by the nucleic acid sequence as set forth in SEQ ID NO: 21) and translocator protein (SipB) (as set forth in SEQ ID NO: 24 and encoded by the nucleic acid sequence as set forth in SEQ ID NO: 23) or fragments thereof (the fusion referred to as S1) (as set forth in SEQ ID NO: 26 and encoded by the nucleic acid sequence as set forth in SEQ ID NO: 25), Salmonella spp. (such as, for example, S. enterica) SPI-2 needle tip protein (SseB) (as set forth in SEQ ID NO: 32 and encoded by the nucleic acid sequence as set forth in SEQ ID NO: 31) and translocator protein (SseC) (as set forth in SEQ ID NO: 34 and encoded by the nucleic acid sequence as set forth in SEQ ID NO: 33) or fragments thereof (the fusion referred to as S2) (as set forth in SEQ ID NO: 36 and encoded by the nucleic acid sequence as set forth in SEQ ID NO: 35. Accordingly, disclosed herein are fusion polypeptides comprising a fusion of a needle tip protein (such as, for example, IpaD, SipD, or SseB) or an antigenic fragment thereof and a translocator protein (such as, for example, IpaB, SipB, or SseC) or an antigenic fragment thereof from a Type III secretion system (T3SS) of a Gram negative bacteria (such as, for example, Salmonella enterica or Shigella spp.)

It is understood and herein contemplated that the arrangement of the polypeptides in a fusion construct can have significant impact on the antigenicity of the fusion construct. Accordingly, in one aspect, disclosed herein are fusion polypeptides, wherein the fusion polypeptide is arranged such that the needle tip protein is 5′ of the translocator protein.

The present invention provides compositions for use in eliciting an immune response and/or vaccinating an individual against Gram negative bacterial infection, and/or against disease symptoms caused by Gram negative bacterial infection. The compositions include one or more substantially purified proteins, polypeptides or antigenic regions thereof as described herein, or substantially purified nucleic acid sequences (e.g. DNA cDNA, RNA, etc.) encoding such proteins, polypeptides or antigenic regions thereof, and a pharmacologically suitable/compatible carrier. By “substantially purified” is meant that the molecule is largely free of other organic molecules, cellular debris, solvents, etc. when tested using standard techniques known to those of skill in the art (e.g. gel electrophoresis, column chromatography, sequencing, mass spectroscopy, etc.). For example, the molecule is generally at least about 50, 55, 60, 65, 70, or 75% pure by wt %, and preferably is at least about 80, 85, 90, 95% or more preferably pure (e.g. 96, 97, 98, 99 or even 100% pure). The preparation of proteins, polypeptides, and peptides as described herein is well-known to those in the art, and includes, for example, recombinant preparation; isolation from a natural source; chemical synthesis; etc. The purification of proteinaceous materials is also known. However, specific exemplary methods for preparing the vaccinating agents utilized in the practice of the invention are described in detail in the Examples section below.

In addition, the composition may contain adjuvants, many of which are known in the art. For example, adjuvants suitable for use in the invention include but are not limited to: bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof. Non-toxic derivatives of LPS include monophosphoryl lipid A (MPL) and 3-O-deacylated MPL (3dMPL). 3dMPL is a mixture of three de-O-acylated monophosphoryl lipid A with 4, 5 or 6 acylated chains. A preferred non-toxic derivative of LPS is 3 De-O-acylated monophosphoryl lipid A. Other non-toxic LPS derivatives include monophosphoryl lipid A mimics, such as aminoalkyl glucosaminide phosphate derivatives, e.g. RC-529.

Lipid A derivatives include derivatives of lipid A from Escherichia coli such as OM-174. Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpG's can include nucleotide modifications/analogs such as phosphorothioate modifications and can be double-stranded or single-stranded, e.g. replacement of guanosine with 2′-deoxy-7-deazaguanosine. The CpG sequence may include, for example, the motif GTCGTT or TTCGTT. The CpG sequence may be specific for inducing a Th1 immune response, such as a CpG-A ODN, or it may be more specific for inducing a B cell response, such a CpG-B ODN, CpG-A and CpG-B ODNs. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”.

Bacterial ADP-ribosylating toxins and detoxified derivatives thereof may be used as adjuvants in the invention. Preferably, the protein is derived from E. coli (e.g. E. coli heat labile enterotoxin “LT”), cholera (“CT”)(Table 1), or pertussis (“PT”).

TABLE 1
Cholera Toxin (CTA1) subunits and sequences

| Subunit | DNA sequence | AA sequence |
|---|---|---|
| Subunit | ATGGTAAAGATAATATTT | MVKIIFVFFIFLSSFSYA |
| A | GTGTTTTTTATTTTCTTA | NDDKLYRADSRPPDEIKQ |
| | TCATCATTTTCATATGCA | SGGLMPRGQSEYFDRGTQ |
| | AATGATGATAAGTTATAT | MNINLYDHARGTQTGFVR |
| | CGGGCAGATTCTAGACCT | HDDGYVSTSISLRSAHLV |
| | CCTGATGAAATAAAGCAG | GQTILSGHSTYYIYVIAT |
| | TCAGGTGGTCTTATGCCA | APNMFNVNDVLGAYSPHP |
| | AGAGGACAGAGTGAGTAC | DEQEVSALGGIPYSQIYG |
| | TTTGACCGAGGTACTCAA | WYRVHFGVLDEQLHRNRG |
| | ATGAATATCAACCTTTAT | YRDRYYSNLDIAPAADGY |
| | GATCATGCAAGAGGAACT | GLAGFPPEHRAWREEPWI |
| | CAGACGGGATTTGTTAGG | HHAPPGCGNAPRSSMSNT |
| | CACGATGATGGATATGTT | CDEKTQSLGVKFLDEYQS |
| | TCCACCTCAATTAGTTTG | KVKRQIFSGYQSDIDTHN |
| | AGAAGTGCCCACTTAGTG | RIKDEL |
| | GGTCAAACTATATTGTCT | (SEQ ID NO: 43) |
| | GGTCATTCTACTTATTAT | |
| | ATATATGTTATAGCCACT | |
| | GCACCCAACATGTTTAAC | |
| | GTTAATGATGTATTAGGG | |
| | GCATACAGTCCTCATCCA | |
| | GATGAACAAGAAGTTTCT | |
| | GCTTTAGGTGGGATTCCA | |
| | TACTCCCAAATATATGGA | |
| | TGGTATCGAGTTCATTTT | |
| | GGGGTGCTTGATGAACAA | |
| | TTACATCGTAATAGGGGC | |
| | TACAGAGATAGATATTAC | |
| | AGTAACTTAGATATTGCT | |
| | CCAGCAGCAGATGGTTAT | |
| | GGATTGGCAGGTTTCCCT | |
| | CCGGAGCATAGAGCTTGG | |
| | AGGGAAGAGCCGTGGATT | |
| | CATCATGCACCGCCGGGT | |
| | TGTGGGAATGCTCCAAGA | |
| | TCATCGATCAGTAATACT | |
| | TGCGATGAAAAAACCCAA | |
| | AGTCTAGGTGTAAAATTC | |
| | CTTGACGAATACCAATCT | |
| | AAAGTTAAAAGACAAATA | |
| | TTTTCAGGCTATCAATCT | |
| | GATATTGATACACATAAT | |
| | AGAATTAAGGATGAATTA | |
| | TGA | |
| | (SEQ ID NO: 42) | |
| Subunit | ATGATTAAATTAAAATTT | MIKLKFGVFFTVLLSSAY |
| B | GGTGTTTTTTTTACAGTT | AHGTPQNITDLCAEYHNT |
| | TTACTATCTTCAGCATAT | QIYTLNDKIFSYTESLAG |
| | GCACATGGAACACCTCAA | KREMAIITFKNGAIFQVE |
| | AATATTACTGATTTGTGT | VPGSQHIDSQKKAIERMK |
| | GCAGAATACCACAACACA | DTLRIAYLTEAKVEKLCV |
| | CAAATATATACGCTAAAT | WNNKTPHAIAAISMAN |
| | GATAAGATATTTTCGTAT | (SEQ ID NO: 45) |
| | ACAGAATCTCTAGCTGGA | |
| | AAAAGAGAGATGGCTATC | |
| | ATTACTTTTAAGAATGGT | |
| | GCAATTTTTCAAGTAGAA | |
| | GTACCAGGTAGTCAACAT | |
| | ATAGATTCACAAAAAAAA | |
| | GCGATTGAAAGGATGAAG | |
| | GATACCCTGAGGATTGCA | |
| | TATCTTACTGAAGCTAAA | |
| | GTCGAAAAGTTATGTGTA | |
| | TGGAATAATAAAACGCCT | |
| | CATGCGATTGCCGCAATT | |
| | AGTATGGCAAATTAA | |
| | (SEQ ID NO: 44) | |

The toxin or toxoid is preferably in the form of a holotoxin, comprising both A and B subunits. Preferably, the A subunit contains a detoxifying mutation; preferably the B subunit is not mutated. More preferably, the adjuvant is a detoxified LT mutant such as LT-K63, LT-R72, and LT-G192. The use of ADP-ribosylating toxins and detoxified derivatives thereof, particularly LT-K63 and LT-R72, is known. Such adjuvants are described, for example, in issued U.S. Pat. No. 8,039,007 (the complete contents of which is hereby incorporated by reference in entirety). Various interleukins may also be used as adjuvants to increase the immune response in a subject. In preferred embodiments, the adjuvant is a mucosal adjuvant such as, for example, the double mutant heat-labile toxin (dmLT) as set forth in SEQ ID NOs: 5 and 6) from enterotoxigenic E. coli or the active moiety thereof known as LTA1 (as set forth in SEQ ID NO: 13 and encoded by the nucleic acid sequence as set forth in SEQ ID NO: 12) and encoded by nor cholera toxin or the active moiety thereof known as CTA1. Accordingly, disclosed herein are fusion polypeptides of any preceding aspect, wherein the fusion further comprises an adjuvant such as, for example, double mutant labile toxin (dmLT) or an antigenic fragment thereof (such as, for example, LTA1 or CTA1) from Enterotoxigenic Escherichia coli. In some aspect, the dmLT or fragment thereof can also be fused to the needle tip protein-translocator protein fusion at the 5′ end. For example, specifically disclosed herein are LTA1-DBF (also referred to herein as L-DBF and as set forth in SEQ ID NO: 15), LTA1-S1 (also referred to herein as L-S1 as set forth in SEQ ID NO: 27 and encoded by the nucleic acid sequence as set forth in SEQ ID NO: 28), LTA1-S2 (also referred to herein as L-S2 as set forth in SEQ ID NO: 38 and encoded by the nucleic acid sequence as set forth in SEQ ID NO: 37), LTA1-SseB (as set forth in SEQ ID NO: 40 and encoded by the nucleic acid sequence as set forth in SEQ ID NO: 39).

It is understood and herein contemplated that the disclosed polypeptides, adjuvants, and acellular vaccine components for use in eliciting an immune response or for treating, inhibiting, or preventing a Gram negative bacterial infection can be administered in compositions such as vaccines as individual polypeptides or as a fusion construct or a combination thereof. Thus, in one aspect, disclosed herein are compositions comprising a T3SA needle tip protein (such as, for example, IpaD, SipD, or SseB) or an antigenic fragment thereof from a Gram negative bacteria (such as, for example, Shigella spp., or Salmonella spp.) and a T3SA translocator protein (such as, for example, IpaB, SipB, or SseC) or an antigenic fragment thereof from a Gram negative bacteria. In one aspect, the composition can comprise the needle tip protein or fragment thereof and the translocator protein or fragment thereof as separate components or as a fusion polypeptide. Also disclosed herein are compositions of any preceding aspect, wherein the composition comprises an adjuvant (such as, for example, cholera toxin, CTA1, dmLT, or LTA1) and/or bacterial toxin protein, such as a pertussis toxoid (PTd). Thus, in one aspect, disclosed herein are vaccines comprising any of the peptides, polypeptides, proteins, fusion peptides, fusion polypeptides, fusion proteins, or compositions disclosed herein. In some embodiments, the vaccine can further comprise an acellular gram negative vaccine or active components thereof.

In some aspects, the composition can be emulsified using a MedImmune Emulsion (ME) or an emulsion of Squalene (8% w/v) and polysorbate 80 (2% w/v weight) (NE). The fusion polypeptide (such as, for example, DBF, S1, S2, L-DBF, L-S1, L-S2) can be at a final concentration of between 0.5 mg/mL and 1.0 mL (including, but not limited to 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.99, 1.0 mg/mL. In some aspects the composition can further comprise Bacterial Enzymatic Combinatorial Chemistry candidate 438 (BECC438).

### 1. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed (such as, for example, IpaD, SipD, SseB, IpaB, SipB, SseC, DBF, S1, or S2) typically have at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### 2. Nucleic Acids

There are a variety of molecules disclosed herein that are nucleic acid based, including for example the nucleic acids that encode, for example IpaD, SipD, SseB, IpaB, SipB, SseC, DBF, S1, or S2 or antigenic fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

### a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine, as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### b) Sequences

There are a variety of sequences related to the protein molecules involved in the signaling pathways disclosed herein, for example IpaD, SipD, SseB, IpaB, SipB, SseC, DBF, S1, or S2, or any of the nucleic acids disclosed herein for making IpaD, SipD, SseB, IpaB, SipB, SseC, DBF, S1, or S2, all of which are encoded by nucleic acids or are nucleic acids. The sequences for the human analogs of these genes, as well as other analogs, and alleles of these genes, and splice variants and other types of variants, are available in a variety of protein and gene databases, including GENBANK®. Those of skill in the art understand how to resolve sequence discrepancies and differences and to adjust the compositions and methods relating to a particular sequence to other related sequences. Primers and/or probes can be designed for any given sequence given the information disclosed herein and known in the art.

### 3. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to about 10⁹ plaque forming units (pfu) per injection but can be as high as about 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, PA 1995.

### 4. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as 22BF into the cell without degradation and include a promoter yielding expression of the gene in the cells into which it is delivered. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### 5. Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, CA, which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed needle tip protein-translocator protein fusion (such as, for example, DBF, S1, or S2) or vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega Biotec, Inc., Madison, WI), as well as other liposomes developed according to procedures standard in the art. In addition, the disclosed nucleic acid or vector can be delivered in vivo by electroporation, the technology for which is available from Genetronics, Inc. (San Diego, CA) as well as by means of a SONOPORATION machine (ImaRx Pharmaceutical Corp., Tucson, AZ).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### 6. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puromycin.

### 7. Peptides

### a) Protein Variants

As discussed herein there are numerous variants of the needle tip protein-translocator protein fusion (such as, for example, IpaD, SipD, SseB, IpaB, SipB, SseC, DBF, S1, or S2) that are known and herein contemplated. In addition, to the known functional strain variants there are derivatives of the needle tip protein and translocator protein which also function in the disclosed methods and compositions. Protein variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than from about 2 to about 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of from about 1 to about 10 amino acid residues; and deletions will range from about 1 to about 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 2 and 3 and are referred to as conservative substitutions.

TABLE 2
Amino Acid Abbreviations

| | |
|---|---|
| Amino Acid | Abbreviations |

| | | |
|---|---|---|
| Alanine | Ala | A |
| Allosoleucine | AIle | |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| Phenylalanine | Phe | F |
| Proline | Pro | P |
| Pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 3
Amino Acid Substitutions

| | Exemplary Conservative |
|---|---|
| Original | Substitutions, others |
| Residue | are known in the art. |
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 3, i.e., selecting residues that differ more significantly in their effect on maintaining (a) the structure of the polypeptide backbone in the area of the substitution, for example as a sheet or helical conformation, (b) the charge or hydrophobicity of the molecule at the target site, or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; (d) a residue having a bulky side chain, e.g., phenylalanine, is substituted for (or by) one not having a side chain, e.g., glycine, in this case, or (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 26 is set forth in SEQ ID NO: 25. It is understood that for this mutation all of the nucleic acid sequences that encode this particular derivative of the DBF are also disclosed. It is also understood that while no amino acid sequence indicates what particular DNA sequence encodes that protein within an organism, where particular variants of a disclosed protein are disclosed herein, the known nucleic acid sequence that encodes that protein in the particular needle tip protein-translocator protein fusion (such as, for example, DBF, S1, or S2) from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 2 and Table 3. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include —CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, —CH₂CH₂—); Spatola et al. Life Sci 38:1243-1249 (1986) (—CH₂—S); Hann J. Chem. Soc Perkin Trans. I 307-314 (1982) (—CH═CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as β-alanine, γ-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 8. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, more preferably from about 7 to about 7.6, and most preferably about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable . . .

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, tri-alkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

In a preferred embodiment, the amount of protein that is administered per dose of vaccine is in the range of from about 0.0001 to about 1000 μg/kg. In one embodiment, the amount is in the range of from about 0.001 to about 1000 μg/kg of body weight of the recipient. In one embodiment, the amount is in the range of from about 0.01 to about 1000 μg/kg of body weight of the recipient. In one embodiment, the amount is in the range of from about 0.01 to about 100 μg/kg of body weight of the recipient. Those of skill in the art will recognize that the precise dosage may vary from situation to situation and from patient to patient, depending on e.g. age, gender, overall health, various genetic factors, and other variables known to those of skill in the art. Dosages are typically determined e.g. in the course of animal and/or human clinical trials as conducted by skilled medical personnel, e.g. physicians or veterinarians.

### C. METHODS OF USING THE COMPOSITIONS

Herein, the protective efficacy of the Shigella spp. tip/translocator fusion, DBF and the Salmonella Spp. tip/translocator fusions S1 and S2, are examined against lethal lung challenge and with complete (sterilizing) clearance of colonizing bacteria. I Our work provides evidence that L-DBF is a self-adjuvanting vaccine that leads to the development of homologous and heterologous cross-protection against Shigella infection. It also appears to elicit an immune response to a highly conserved pair of T3SS proteins with a cytokine profile that is tailored toward clearance of Shigella from mucosal sites. Such cross-protective immunogenicity is not disrupted by prior exposure to the target pathogen, thereby indicating that L-DBF can be used in those areas where shigellosis cases are common. t has been reported that the DBF fusion vaccine does not elicit a serum antibody response in humans during the course of natural infection and is not a protective antigen in mice. Nevertheless, as shown herein, protective and sterilizing immunity can be obtained with the compositions disclosed herein.

Thus, in one aspect, disclosed herein are methods of eliciting an immune response in a subject to a Gram negative bacteria (such as, for example, Shigella spp. or Salmonella spp) comprising administering to the subject the fusion polypeptides, compositions, or vaccines disclosed herein. Accordingly, in one aspect, disclosed herein are methods of eliciting an immune response against at least one Gram negative bacteria serovar in a subject in need thereof, comprising administering to the subject a composition comprising at least one needle tip protein or a fragment thereof and/or at least one translocator protein or a fragment thereof; wherein said composition is administered in an amount sufficient to elicit an immune response to said at least one Gram negative bacteria serovar in said subject. In one aspect, the immune response elicited provides sterilizing immunity to the infectious bacterium.

As shown herein, unlike other vaccines, the efficacy and protection conferred by the fusion polypeptides disclosed herein (i.e., DBF, S1, and S2) including fusions comprising LTA-1 (such as, for example, SEQ ID NO: 16 (L-DBF), SEQ ID NO: 28 (L-S1), and SEQ ID NO: 38 (L-S2) or any composition comprising said fusion polypeptides, has a multimeric presentation and, thus, is not dependent of the serotype of any prior infection. That is, immunological protection is conferred regardless of the serotype from any prior exposure. Therefore, disclosed herein are methods of eliciting an immune response in a subject to a Gram negative bacteria (such as, for example, Shigella spp. or Salmonella spp) comprising administering to the subject the fusion polypeptides, compositions, or vaccines disclosed herein wherein the subject has a prior exposure to the Gram negative bacteria that is the same or different serotype than the immunizing polypeptide and/or the same or different from the serotype of the Gram negative bacteria for which protection is sought.

As can be appreciated by the skilled artisan, the methods of eliciting an immune response can be used for the purpose of treating, inhibiting, or preventing an infection of a Gram negative bacteria (such as, for example, Shigella spp. or Salmonella spp). Thus, in one aspect, disclosed herein are methods of treating, inhibiting, or preventing an infection of a Gram negative bacteria in a subject comprising administering to the subject a therapeutic amount of any of the fusion polypeptides, compositions, or vaccines disclosed herein. As one goal of any vaccine is not only to prevent infection or reducing the severity of disease in the individual receiving the vaccine, but also to prevent further transmission of the infectious agent (sterilizing immunity), it is understood and herein contemplated that the disclosed methods of treatment, inhibition, or preventing an infection can further comprise inhibiting and/or preventing colony formation of the bacteria and/or transmission of the bacteria to another subject.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

a) Use of a T3SS Needle Tip/Translocator Protein Fusion as a Protective Antigen Against B. pertussis:

The dominant antigen eliciting protection against Gram-negative pathogens is LPS, which confers O-antigen serotype specificity. The initial project focused on Shigella, however, there are at least 58 distinct Shigella serotypes. This reduces broad-spectrum efficacy for live, attenuated and whole, killed vaccines, which tend to be somatic antigen driven. IpaD and IpaB are the surface-localized needle tip and first translocator proteins of the T3SA, respectively. They are essential for virulence in all Shigella, are >98% conserved across all Shigella species, and provide serotype-independent protection. When IpaD+IpaB+dmLT was given IN to mice, the formulation was about 80-90% protective against lethal challenge by homologous and heterologous Shigella spp. (Table 4). To reduce the production cost, IpaD and IpaB were genetically fused to make DBF. Not only did the DBF provide protection against lethal challenge, it also unexpectedly increased the cell-mediated immunity, most notably the IL-17 and IFN-γ responses. When the Salmonella enterica tip and first translocator proteins of the T3SSs of SPI-1 and SPI-2 (Salmonella Pathogenicity Islands 1 and 2) were fused, about 70% protection against lethal challenge by two S. enterica serovars was observed when both fusion proteins were administered simultaneously (Table 4).

TABLE 4
Protective efficacy of tip/translocator fusions
against challenge by appropriate pathogen.

| Vaccine | Protection (%) | Pathogen |
|---|---|---|
| DBF + dmLT | 80-90 | S. flexneri, S. sonnei |
| S1S2 + MPL (IM) | 70 | S. Typhimurium, S. Enteritidis |
| 22BF + dmLT | 100 | B. bronchiseptica |

Mice (n = 10) were vaccinated 3 times biweekly with the vaccine and then challenged with indicated pathogen at day 56. Protection is indicated as percent survival after 21 days.

It is shown herein that the use of a broad, serotype-independent subunit vaccine against Shigella spp. and S. enterica serotypes. These vaccines are based on the fusion of the T3SA tip and first translocator proteins, which are highly conserved within a given bacterial genus. The DBF has also been shown to protect monkeys from severe diarrhea and S. enterica S1 or S2 fusions are protective in a bovine calf model. A genetic fusion of the Shigella T3SA tip/translocator system, DBF, was generated which protects 80-90% of the mice challenged with a lethal dose of S. flexneri and S. sonnei. Similarly, a like fusion from Salmonells (S1 and S2 fusions) offered 70% protection of mice challenged with a lethal dose of S. typhimurium and S. enteritidis.

### Example 2: LTA-1 Fusion

LTA1 is the active moiety of lethal toxin from Enterotoxigenic E. coli (ETEC). The activity of the LTA1 is required for the adjuvant activity of dmLT. The double mutants are in the region usually targeted by a protease to allow A1 to traffic to the cytoplasm of intestinal cells to cause the secretory diarrhea. Without the protease the LT still has some activation of cAMP. Likewise, LTA1 remains active.

### a) LTA1-Fusions:

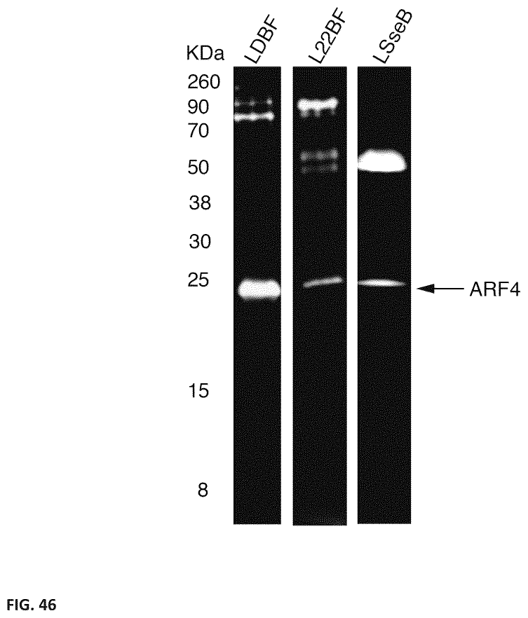

The LTA1-fusions were expressed in a manner similar to the fusion alone. The LTA1 sequence was inserted 5′ to the start of the each fusion. Some of the LTA1-fusions required a small linker between the LTA1 and fusion in order for protein production to occur. LTA1-DBF (also referred to herein as L-DBF), LTA1-S1 (also referred to herein as L-S1), and LTA1-S2 (also referred to herein as L-S2) were produced. One of the assays that appear to be required for adjuvant activity is the ability to ADP ribosylate ARF4. The ADP ribosylation assay was performed with the LTA1-fusions. In the assay, ADPr was biotin conjugated and when mixed with LTA1 and rARF4, the LTA1 transferred the biot-ADPr to rARF4. The biotin was then detected with Streptavidin-IR800 (FIG. 46).

### b) LTA1-DBF Immune Response:

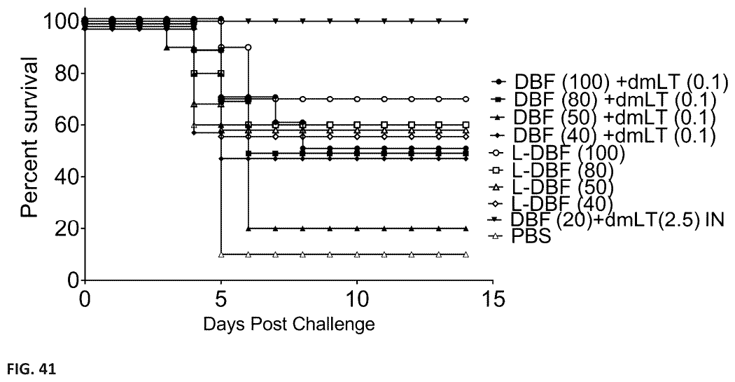

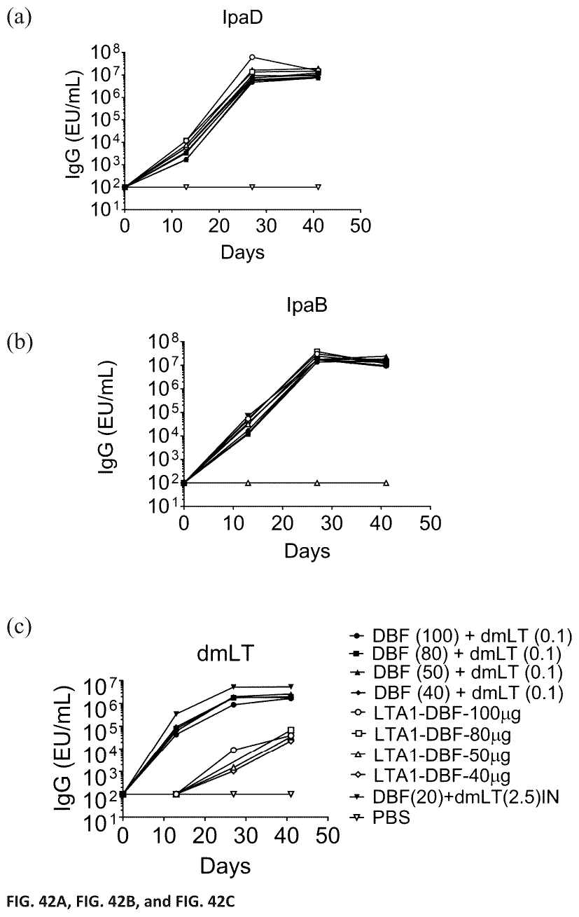

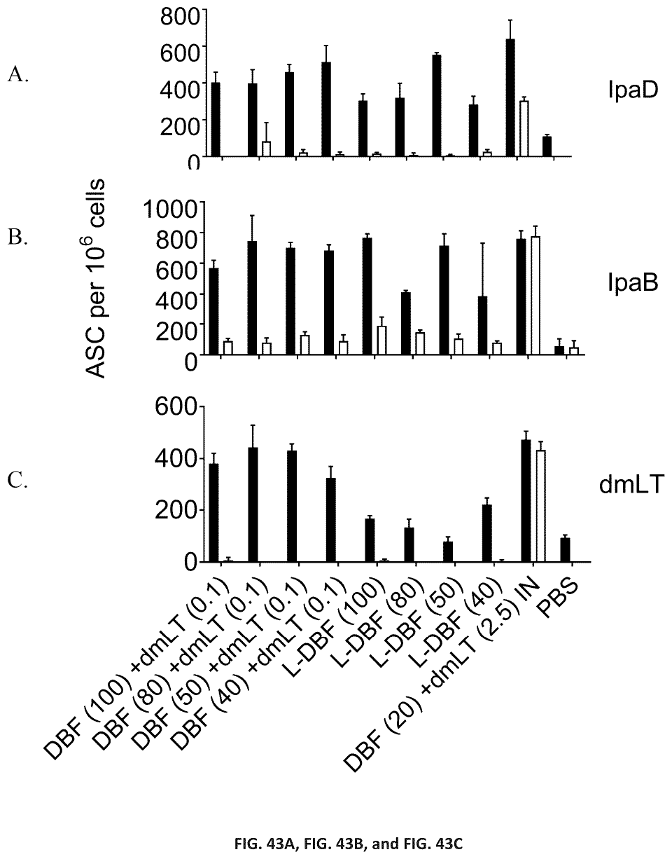

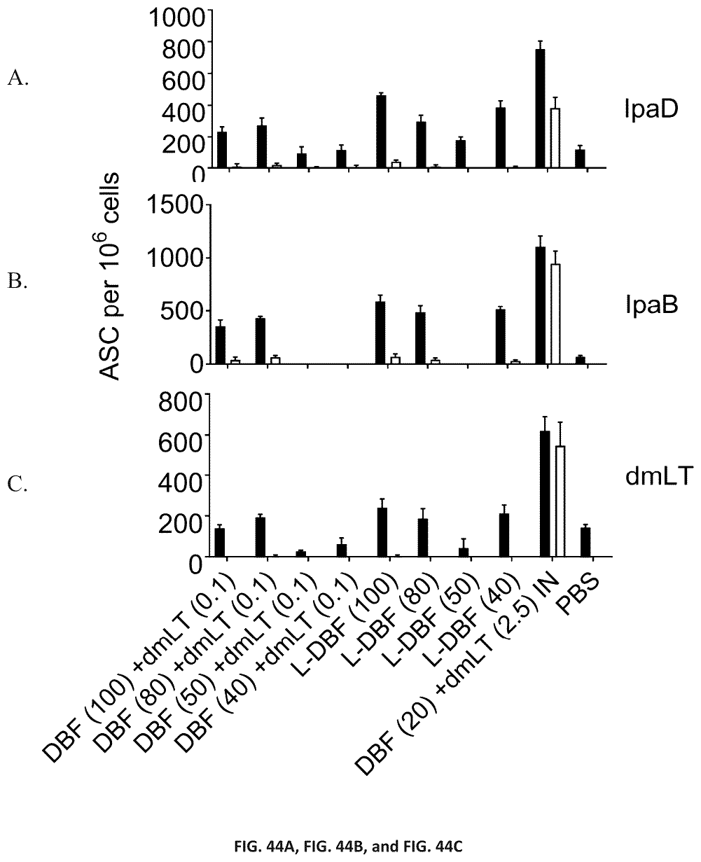

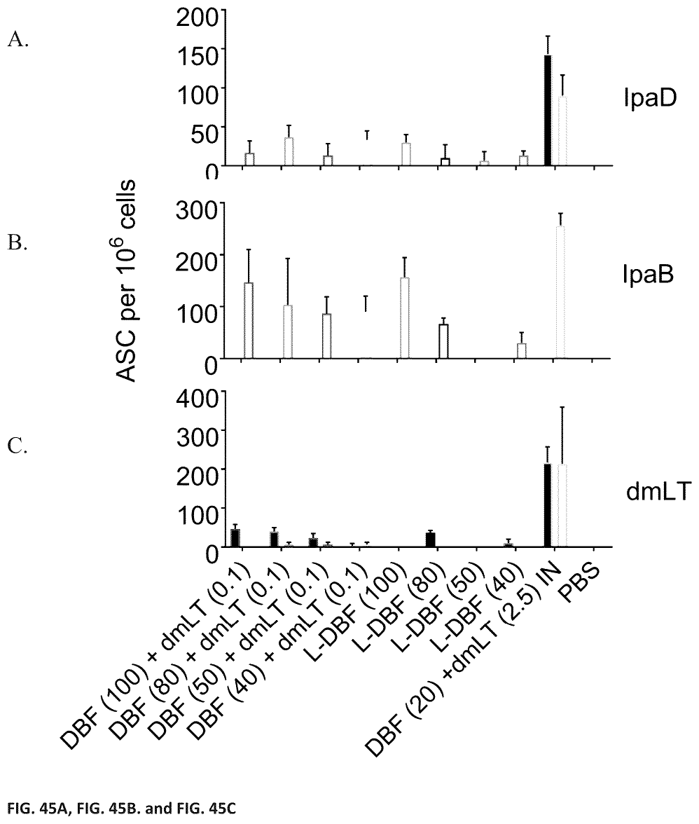

When the kinetics of the IgG titer was examined, responses against IpaD and IpaB were essentially the same. Mice from FIG. 41 were bled prior to vaccination and on day 42. Sera were assessed for anti-IpaD, -IpaB and -dmLT IgG. The lower IgG titers of the LTA1 samples can be attributed to the lower recognition of the dmLT on the well for the LTA1 samples vs the samples from mice vaccinated with dmLT (FIG. 42). No IgA was detected in the fecal samples of the mice vaccinated IM, but was detectable in mice vaccinated IN with DBF+dmLT. Antibody-secreting cells (ASCs) present in the bone marrow (FIG. 43), spleen (FIG. 44), and lungs (FIG. 45) were also stimulated with IpaD, IpaB and dmLT. In each compartment, anti-IpaD, anti-IpaB, and anti-dmLT IgG ASCs could be detected. Interestingly, IgA ASCs could also be detected in the bone marrow against all dilutions of LTA1-DBF and resemble a curve similar to the dose escalation. A similar phenomenon was seen in the IgA ASCs from the lungs, but less pronounced.

### c) LTA1-DBF Purification.

The yield of LTA1-DBF was very low. Therefore, a linker was inserted in the DNA sequence between LTA1 and DBF to encode GSAAS (Seq. ID No. 14). The mother plasmid was Novagen's pACYCDuet-1. The translocator for each fusion cannot be made without its cognate chaperone. Therefore, the complex of LTA1-DBF/Histag-IpgC (IPG chaperone comprises the nucleic acid sequence as set forth in SEQ ID NO: 10 which encodes the amino acid sequence as set forth in SEQ ID NO: 11) was produced from the plasmid pACYC-His-IpgC-LTA1-GSAAS-DBF where the ipgC gene was inserted into the BamHI/HindIII sites allowing for expression of His-tag IpgC and LTA1-GSAAS-DBF (nucleic acid sequence as set forth in SEQ ID NO: 15 and amino acid sequence as set forth in SEQ ID NO: 16)) was inserted at the NdeI-XhoI site. The DBF sequence had a 3′ stop codon prior to the XhoI restriction site.

pACYC-His-IpgC-LTA1-GSAAS-DBF was transformed into Tuner cells. A small overnight culture of LB+Chloramphenicol (Cm) that had been inoculated with the freezer stock of the cells was transferred to 8 L TB, and grown at 37° C. until OD=1-1.5, add 0.5 mM IPTG with 20 μg/liter AEBSF, 16C overnight, harvested at 4000 rpm for 15 min at 4° C., and resuspended in IMAC binding buffer. The cells were frozen at −80° C. until ready for purification. After thawing the suspension was sonicated at 70% amplitude for 3-4 min, 15 s on, 30 s off, clarified by centrifugation at 13000 rpm for 30 min at 4° C. and decanted to obtain supernatant.

IMAC purification with 5 ml NiNTA FF crude column on AKTA was as follows: (1) equilibrate column with 5CV binding buffer (20 mM Tris, 500 mM NaCl, 5 mM Imidazole pH 7.9), (2) load supernatant on column, collect FT in outlet1, (3) wash with binding buffer for 30CV, (4) elute with linear 0-60% elution buffer (20 mM Tris, 500 mM NaCl, 500 mM Imidazole pH 7.9) for 10CV, (5) elute with 60% elution buffer for 2CV, (6) wash column with 100% elution buffer for 3CV, (7) re-equilibrate column with 5CV binding buffer for 5CV.

HIC purification of the protein was as follows: Dilute pooled fraction into equal volume of 2×HIC binding buffer (50 mM Sodium Phosphate (dibasic), 1M Ammonium Sulfate, pH 7.0). Purify with 5 ml HIC Phenyl HP column: (1) equilibrate column with 5CV binding buffer, (2) load diluted sample on column, collect FT in outlet1, (3) wash with binding buffer for 5CV, (4) elute with linear 0-100% elution buffer (5 mM Sodium Phosphate (dibasic), pH 7.0) for 40CV, (6) elute with 100% elution buffer for 6CV, (7) wash column with 100% elution buffer for 3CV, (8) reequilibrate column with binding buffer for 5CV.

Pooled fractions were dialyzed in 4 L Q binding buffer for 2 hrs, exchanged buffer, and then dialyzed overnight.

Purification using a 5 mL Q FF columns on AKTA was as follows: (1) equilibrate column with 5CV binding buffer (50 mM Tris, pH 8.0), (2) load dialyzed sample on column, collect FT in outlet1, (3) wash with binding buffer for 5CV, (4) elute with linear 0-30% elution buffer (50 mM Tris, 1M NaCl, pH 8.0) for 20CV, (6) elute with 100% elution buffer for 5CV, (7) wash column with 100% elution buffer for 3CV, (8) re-equilibrate column with binding buffer for 5CV.

To facilitate final IMAC purification 8×IMAC binding buffer (NO Imidazole) was added to pooled fractions to obtain 1× and then LDAO to 0.05% was added

Purification by LDAO IMAC using 5 ml NiNTA FF was as follows: (1) equilibrate column with 5CV LDAO (20 mM Tris, 500 mM NaCl, 0.05% LDAO pH 7.9) binding buffer, (2) load supernatant on column, fractionate FT, (3) wash with binding buffer for 5CV, fractionate, (4) wash with 3% LDAO elution buffer (20 mM Tris, 500 mM NaCl, 500 mM Imidazole, 0.005% LDAO pH 7.9) for 5CV, fractionate (5) elute with 6% LDAO elution buffer for 6.65CV, fractionate (6) elute with 100% LDAO elution buffer for 5CV, (8) re-equilibrate column with 5CV binding buffer for 5CV.

Pooled samples were dialyzed in 4 L PBS+0.005% LDAO, exchanged buffer after 2 hrs, and then dialyzed overnight.

### Example 3: L-DBF Elicits Cross Protection Against Different Serotypes of Shigella Spp

Shigella spp. include S. dysenteriae (Group A), S. flexneri (Group B), S. boydii (Group C), and S. sonnei (Group D), which are further divided into more than 50 serotypes based on O-antigen composition. S. flexneri is the primary cause of endemic diarrhea in developing countries where there is limited access to hygienic resources, whereas S. sonnei is the leading cause of illness in developed countries. In addition, S. flexneri is responsible for a greater number of total deaths from shigellosis than the other species, with serotypes 1b, 2a, 3a, 4a, and 6 commonly found in less developed countries and serotype 2a dominant in moderately developed countries. Recent studies have shown that S. sonnei infections are increasing and replacing S. flexneri as a cause of shigellosis in areas as they undergo modernization, evincing the need for a serotype-independent Shigella vaccine.

Although there has been a reduction in the incidence of shigellosis globally due to improved sanitation, the rise of antimicrobial resistance in Shigella spp. warrants the development of a vaccine against this pathogen. At present, there is no licensed Shigella spp. vaccine, however, some killed cell and live-attenuated vaccines are currently in clinical trials. Unfortunately, the lack of cross-protection, strict storage conditions, and potential risks of contamination limit their use in developing countries. To solve these issues, subunit vaccines, especially those utilizing proteins from the type three secretion system (T3SS), have been widely researched.

The T3SS is a required virulence factor for the shigellae. The T3SS apparatus (T3SA) tip protein, IpaD, and translocator, IpaB, are highly conserved among Shigella spp., making them excellent targets for the development of a serotype-independent subunit vaccine. Research in our lab has established that these two proteins, with the adjuvant dmLT (double-mutant heat-labile enterotoxin from enterotoxigenic E. coli (ETEC)), can elicit cross protection against S. flexneri and S. sonnei when delivered intranasally (IN). To reduce production costs, we made DBF, a genetic fusion of IpaD and IpaB. DBF adjuvanted with dmLT induced comparable immune responses in both B and T cells to those stimulated by the mixture of IpaD and IpaB. Most importantly, DBF admixed with dmLT and administered IN protected mice in lethal challenges with the homologous S. flexneri, from which the IpaD and IpaB sequences are derived, and in challenges with the heterologous S. sonnei and S. dysenteriae.

The dmLT, an AB5 toxoid which induce anti-LT response, retains the native ADP-ribosyltransferase activity that induces a strong Th17 response. Studies have shown that pre-existing antibodies to dmLT did not disturb its adjuvanticity to a new antigen. This suggested that anti-LT antibodies from pre-exposures of ETEC, which commonly happen in developing countries, would not affect the adjuvant effects of LTA1. Th17 responses are known to be especially important for protection against mucosal pathogens, including Shigella. Unfortunately, recent studies showed that dmLT, when delivered IN, can cause Bell's palsy, however, the development of Bell's palsy from dmLT is related to the ability of the B subunit to bind to the gangliosides of neuron cells. It is the LTA1 (heat-labile enterotoxin A1) portion of the A subunit that is responsible for generating the Th17 response. To simplify and lower the costs of producing a Shigella spp. vaccine for use in developing nations, we genetically fused LTA1 to DBF to create a monomeric adjuvant-antigen conjugate called L-DBF.

In this study, we demonstrate that IN administration of L-DBF protects mice against a lethal pulmonary challenge with S. flexneri 2a, and this protection is associated with significant Th1 and Th17 responses. Additionally, we show that IN immunization with L-DBF protects mice against lethal challenges with heterologous S. flexneri 1b, S. flexneri 3a, S. flexneri 6, and S. sonnei. These results show that L-DBF elicits broad protective efficacy against multiple Shigella serotypes and is thus a viable vaccine candidate against shigellosis.

### a) Materials and Methods

### (1) Materials

pACYCDuet-1 plasmid, ligation mix and competent E. coli were from EMD Millipore (Billerica, MA). Restriction endonucleases were from New England Biolabs (Ipswich, MA). Chromatography columns were from GE Healthcare (Piscataway, NJ). All other reagents were from Sigma or Fisher Scientific. dmLT was from J. Clements and E. Norton (Tulane School of Medicine, New Orleans, LA). S. flexneri 2a 2457T was from A. T. Maurelli (University of Florida, Gainesville, FL). S. flexneri 1b, S. flexneri 3a, S. flexneri 6, and S. sonnei were from Eileen Barry (University of Maryland School of Medicine, Baltimore, MD).

### (2) Protein Production

Production of IpaD, IpaB and DBF have been described previously. To produce LTA1-GSAAS-DBF (L-DBF), eltA1 (the coding sequence for LTA1) was cloned in-frame with the linker gggtccgcggcatcc 5′ to ipaD in the IpaD-IpaB+IpgC/pACYCDuet-1 plasmid. The resulting plasmid (eltA1-ipaD-ipaB/ipgC//pACYCDuet-1) was used to transform E. coli Tuner (DE3) for co-expression of L-DBF and the IpgC chaperone with the latter possessing a His affinity tagee (HT-IpgC). The transformed bacteria were grown in a fed-batch mode using a 10 L bioreactor (Labfors 5, Infors USA Inc., MD) equipped with polarographic dissolved oxygen probe (pO2), pH probe (Hamilton Company) and advanced fermentation software. Materials were prepared as per the manufacturer's specifications. Briefly, pre-culture was prepared by inoculating a 25-μl aliquot of frozen glycerol stock into 50 mL of Terrific broth (TB) supplemented with chloramphenicol (34 μg/ml) and allowed to grow overnight at 30° C. with shaking at 200 rpm. The inoculum was made by transferring cells from the pre-culture to 1 L of TB with the same antibiotic and grown at 30° C. until reaching an A600 of ˜2.0. Then, ˜800 mL of inoculum was transferred to the sterilized bioreactor containing 9 L of TB containing chloramphenicol. The culture was maintained at 30° C. and pH 7, and stirrer speed, gas mix, and gas flow were adjusted to maintain pO2 (30%). Protein expression was induced by addition of IPTG to 1 mM when the culture reached an A600 of ˜25. After 3 h, bacteria were collected by centrifugation, washed and resuspended in IMAC binding buffer (20 mM Tris-HCl PH 7.9, 500 mM NaCl, 10 mM imidazole) with 0.1 mM AEBSF Protease Inhibitor and lysed using a microfluidizer at 18,000 psi with three passes. The cellular debris was removed by centrifugation at 10,000×g for 30 min and loaded onto a 5 ml HisTrap FF column. The L-DBF/HT-IpgC was eluted with IMAC elution buffer (20 mM Tris-HCl pH 8.0, 500 mM NaCl, 500 mM imidazole), dialyzed into 50 mM Tris-HCl pH 8.0, and loaded onto a HiTrap Q FF column. The complex was eluted using a gradient of 50 mM Tris-HCl pH 8 containing IM NaCl. Lauryldimethylamine oxide (LDAO) was then added to a final concentration of 0.1% to release the HT-IpgC. When the LDAO-treated L-DBF/HT-IpgC complex was passed over an IMAC column, the L-DBF was collected in the flow-through with the HT-IpgC being retained on the IMAC column. Finally, L-DBF was dialyzed into 20 mM phosphate, pH 7.2, with 150 mM NaCl (PBS) with 0.05% LDAO and stored at −80° C. LPS levels were determined using a NexGen PTS with EndoSafe cartridges (Charles River Laboratories, Wilmington, MA). All proteins had LPS levels <5 Endotoxin units/mg.

### (3) ADP-Ribosylation Assay

LTA1 from ETEC labile-toxin possesses ADP-ribosyltransferase (ADPr) activity, which is required for adjuvanticity. To assay for L-DBF ADPr activity, L-DBF was added to ADPr buffer (50 mM Tris-HCl pH 7.5, 1 mM EDTA, 1 mM DTT) to a concentration of 1.7 μM, with or without 1.7 UM ARF4, the LTA1 allosteric activator protein. Biotinylated-NAD+ was then added to a concentration of 8.3 UM and the mixture incubated at 37° C. for one hour. The reaction mixtures were then subjected to SDS-PAGE and then electroblotted onto a nitrocellulose membrane. The membrane was then incubated in TBS buffer containing IRDye 800CW streptavidin (LI-COR, Lincoln, NE), washed, and imaged on a LI-COR Odyssey CLx gel scanner.

### (4) Immunization of Mice

The mouse animal protocols were reviewed and approved by the University of Kansas Institutional Animal Care and Use Committee Practices (protocol AUS 222-01). Female 6-8 weeks BALB/c mice were used in this study (n=10/group). For the initial intranasal (IN) vaccination trial, 20 μg DBF+2.5 μg dmLT and 25 μg L-DBF were prepared in 30 μl per mouse for each IN vaccination. For the intradermal (ID) trial, 100, 250 and 500 ng L-DBF were diluted to 50 μl per mouse. For the intramuscular (IM) trial, 80 μg L-DBF+2.5 μg dmLT and 80 μg L-DBF were prepared in 30 μl volume for each mouse. For the IN dose escalation (n=14/group; 10 for challenge and 4 for pre-challenge immune response assessment), 1, 10, and 25 μg L-DBF were prepared in 30 μl volume for each mouse. In a separate experiment, (n=14/group; 10 for challenge and 4 for pre-challenge immune response assessment), 15, and 25 μg L-DBF were prepared in 30 μl volume for each mouse. To test for cross-protection, 25 μg L-DBF and PBS alone were prepared in 30 μl. All mice, regardless of route, were immunized on days 0, 14 and 28.

### (5) Shigella Challenge Studies

Shigella challenge strains were streaked onto tryptic soy agar containing 0.025% Congo red and incubated at 37° C. overnight and subcultured in tryptic soy broth (TSB) at 37° C. until A600 reached 1.0. Bacteria were harvested by centrifugation, resuspended in PBS and diluted to the desired concentration in a 30 μl volume for IN challenge. In the cross-protection study, L-DBF 25 μg and PBS vaccinated mice (n=10 of each group/serotype) were challenged on day 56 with S. flexneri 2a (6×10⁶ CFU/30 μl), S. flexneri 3a (1×10⁶ CFU/30 μl), S. flexneri 6 (1×10⁶ CFU/30 μl), S. flexneri 1b (4×10⁶ CFU/30 μl), or S. sonnei (1×10⁶ CFU/30 μl). Mice were monitored twice a day for weight loss and health score for two weeks. Mice were euthanized if their weight loss exceeded 25% of their original weight for more than 72 h or their blood glucose reached ≤100 mg/dL with poor health scores.

### (6) IgG and IgA ELISAs

Blood and feces were collected on days 0, 13, 27, 41, and 55 for antibody detection during the immunization, as described previously with minor modifications. Microtiter wells were coated with 100 ng IpaB or IpaD in 100 μl PBS, incubated at 37° C. for 3 h and then blocked overnight with 10% nonfat dry milk in PBS. Sera were added to the wells in duplicates as the primary antibody for 2 h incubation at 37° C. After washing with PBS-0.05% Tween, HRP-conjugated secondary antibody (IgG(H+L), 1:1000; IgA, 1:500) was added and incubated for 1 h at 37° C. After an additional wash, OPD substrate (o-phenylenediamine dihydrochloride) was added and the resulting signal detected at 490 nm. Endpoint titers were determined by fitting antibody titrations to a five-parameter logistic model.

### (7) IFN-γ or IL-17A ELISpots Analysis

Single cell suspensions from mouse spleens and lungs were isolated using Spleen or Lung Dissociation Kit (Miltenyi Biotec, Inc), and were incubated for 24 h at 37° C. in the presence of 5 μg/ml IpaB or IpaD in plates coated with antibodies against IFN-γ or IL-17A using a FluoroSpot assay as per manufacture's specifications (Cellular Technology Limited). The cytokine secreting cells were quantified using a CTL immunospot reader.

### (8) Cytokine Determinations

Splenocytes and lung cells were incubated with 10 μg/ml IpaB, IpaD or PBS for 48 h at 37° C. Supernatants were collected and analyzed with U-PLEX kits for cytokines: IFN-γ, IL-17A, IL-6, and TNF-α. Cytokine concentrations were determined using an MSD plate reader with associated analytical software (Meso Scale Discovery, Rockville, MD).

### (9) Statistics

Graphs were generated using GraphPad Prism 9.0.1. ELISpot and cytokine secretion were rescaled to the range between zero and one using the minimum-maximum (min-max) normalization equation [Ynormal=(Yorigin−Ymin)/(Ymax−Ymin)] for the purpose of plotting comparative data. The significance of differences among treatment groups was determined using ANOVA. Post-hoc comparisons of unvaccinated (PBS) mice with antigen vaccinated mice were made with Dunnett's test in R. A p-value of less than 0.05 was considered significant for all comparisons (*p<0.05; **p<0.01; ***p<0.001). For bacterial challenges, vaccinated groups were compared to PBS using Log-rank (Mantel-Cox) tests in GraphPad Prism.

### b) Results

### (1) The LTA1 Domain of L-DBF Retains Its ADP-Ribosyltransferase Activity

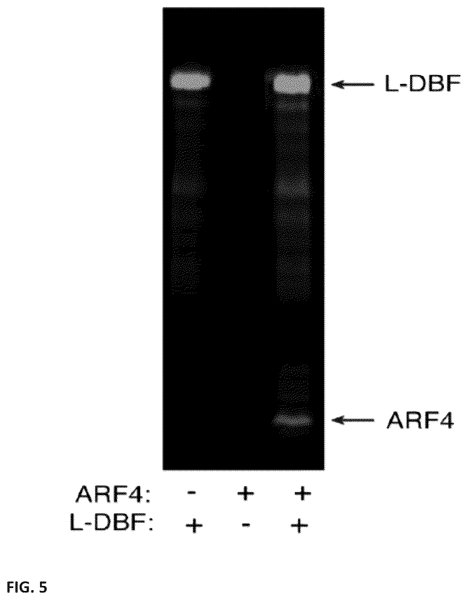

When incubated with biotin-labeled NAD+, L-DBF was found to conjugate biotinylated ADP-ribose to itself and to the LTA1 allosteric activator protein ARF4 (FIG. 5). This result does not in itself guarantee that the LTA1 domain retains adjuvanticity, however, the absence of enzymatic activity would have precluded the use of L-DBF as a self-adjuvanting vaccine candidate. It should be noted that recombinant LTA1 sequesters to E. coli inclusion bodies and must be refolded after solubilization in a denaturing agent such as urea. Because this would increase the difficulties encountered in formulation, the use of purified LTA1 with DBF was not included as a vaccine comparator in the studies that follow.

(2) Mice Immunized Intranasally With L-DBF Demonstrated Similar Protection Against a Lethal Shigella Challenge as Those Treated With DBF+dmLT

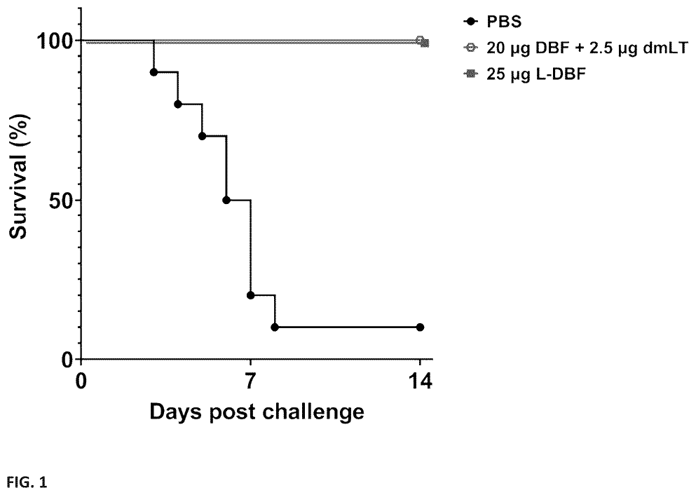

To demonstrate that L-DBF could protect mice against an S. flexneri challenge as well as DBF+dmLT does, we vaccinated mice intranasally (IN) with 20 μg DBF+2.5 μg dmLT, 25 μg L-DBF or PBS. After three vaccinations, the mice were challenged with of 6×10⁶ CFU S. flexneri 2a 2457T (FIG. 1). While 90% of the mice vaccinated with PBS died, all mice vaccinated with either DBF+dmLT or L-DBF survived (FIG. 1). These results show that L-DBF has protective efficacy equivalent to that of DBF with dmLT and is a viable self-adjuvanting vaccine candidate.

(3) Mice Immunized Intramuscularly or Intradermally with L-DBF are not Protected Against a Lethal Shigella Challenge

To determine whether other vaccination routes would also be protective, we immunized mice with L-DBF intramuscularly (IM) and intradermally (ID). Mice were vaccinated IM with 80 μg L-DBF or 80 μg L-DBF+2.5 μg dmLT, or ID with 100, 250 and 500 ng L-DBF. An additional 2.5 μg dmLT was added to one IM group to boost the production of IL-17A. As a positive control, mice were vaccinated IN with DBF+dmLT or L-DBF. In contrast to IN vaccinated mice, only 56% and <30% of the mice vaccinated IM and ID, respectively, survived (Table 5). The inclusion of additional adjuvant (dmLT) did not improve the protective capacity of the L-DBF administered IM (Table 5). Because the WHO has established >60% vaccine efficacy as a targeted cut-off value for a preferred Shigella spp. vaccine candidate, the IM and ID routes are thus considered not viable for L-DBF.

TABLE 5
Vaccine efficacy of L-DBF administered via different routes

| Vaccination regimen | VE |
|---|---|
| 20 μg DBF + 2.5 μg dmLT IN | 100% |
| 25 μg L-DBF IN | 100% |
| 80 μg L-DBF IM | 56% |
| 80 μg L-DBF + 2.5 μg dmLT IM | 56% |
| 500 ng L-DBF ID | 30% |
| 250 ng L-DBF ID | 0% |
| 100 ng L-DBF ID | 20% |

*VE (vaccine efficacy) = 1 − ARV/ARU.
Mice (n = 10) were vaccinated with the indicated formulation and via the indicated route on days 0, 14 and 28. They were then challenged with 6 × 10⁶ CFU/30 μl S. flexneri 2a 2457T on day 56. Vaccine Efficacy (VE) is shown where VE = 1 − Attack Rate Vaccinated/Attack Rate Unvaccinated.

(4) Intranasal Immunization with L-DBF Protects Mice from Five Different Shigella Serotypes

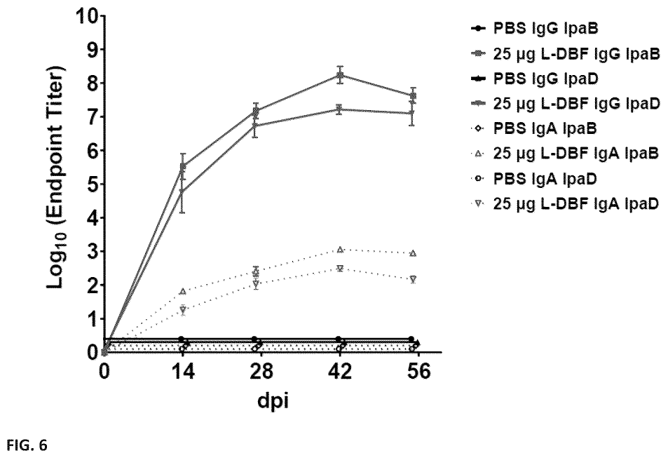

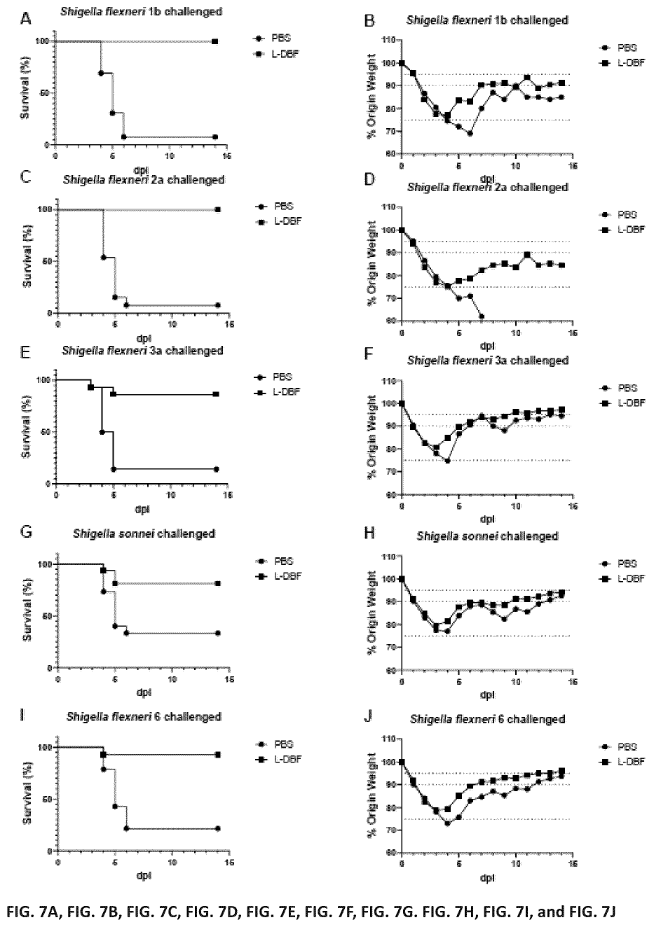

Five groups of ten mice were vaccinated three times IN with 25 μg L-DBF. An additional five groups were vaccinated with PBS as negative controls. Serum IgG and fecal IgA titers against IpaB and IpaD were then assessed (FIG. 6). All vaccinated mice had significantly higher serum anti-IpaB and anti-IpaD IgG and fecal IgA titers when compared to the negative control groups. One group of each of the L-DBF or PBS vaccinated mice were then challenged on day 56 with S. flexneri 2a (6×10⁶ CFU), S. flexneri 3a (1×10⁶ CFU), S. flexneri 6 (1×10⁶ CFU), S. flexneri 1b (4×10⁶ CFU), or S. sonnei 53G (1×10⁶ CFU), based on the LD50 of each strain. All vaccinated groups showed >83% survival when challenged with an S. flexneri serotype, while protection against S. sonnei was 68% (Table 6 and FIG. 7). PBS vaccinated mice also showed greater weight loss and slower recovery than the L-DBF vaccinated mice (FIG. 7). The somewhat rapid weight gain seen for some of the PBS groups was a result of a small number of surviving mice, which skewed the average weight upward at later time points in the experiment. As with IpaB+IpaD or DBF, the protection seen here demonstrates the broad serotype-independent efficacy of L-DBF. Notably, there are no reports of any LPS-based vaccine formulations that provides protection against five Shigella serotypes simultaneously. Moreover, anti-Shigella immunity is serotype specific.

TABLE 6
Vaccine efficacy of L-DBF against Shigella spp. serotypes

| Challenge strains | ARV | ARU | VE |
|---|---|---|---|
| Shigella flexneri 2a | 0% | 92% | 100% |
| (6 × 10⁶ CFU) | | | |
| Shigella flexneri 3a | 14% | 86% | 83% |
| (1 × 10⁶ CFU) | | | |
| Shigella flexneri 6 | 8% | 85% | 91% |
| (1 × 10⁶ CFU) | | | |
| Shigella flexneri 1b | 0% | 92% | 100% |
| (4 × 10⁶ CFU) | | | |
| Shigella sonnei 53G | 21% | 67% | 68% |
| (1 × 10⁶ CFU) | | | |

Mice (n = 10/group) were vaccinated with 25 μg L-DBF (ARV) or PBS (ARU) and challenged with the indicated serotype at the indicated dose. Vaccine Efficacy (VE) is shown where VE = 1 − Attack Rate Vaccinated (ARV)/Attack Rate Unvaccinated (ARU).

### (5) Intranasal L-DBF Stimulates Dose Dependent Immune Responses and Protection in Mice

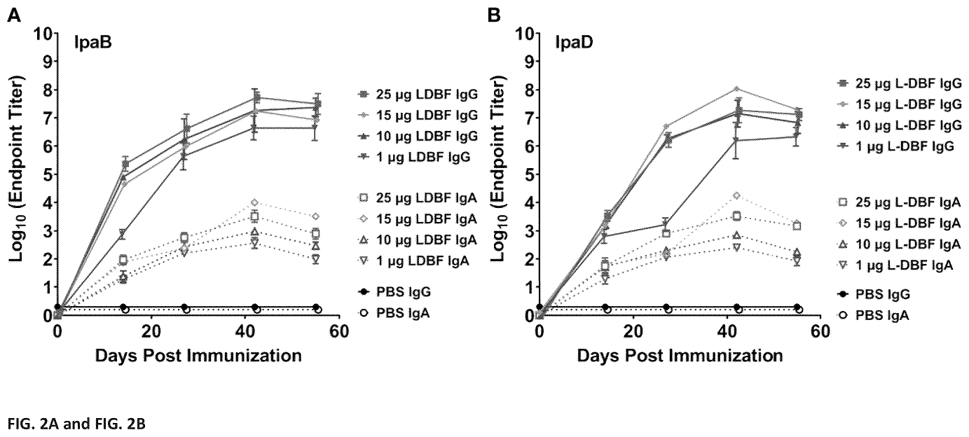

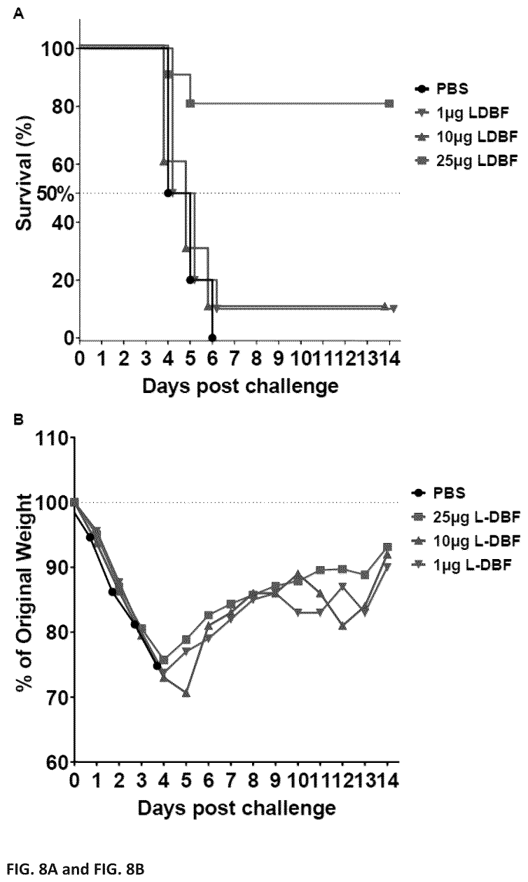

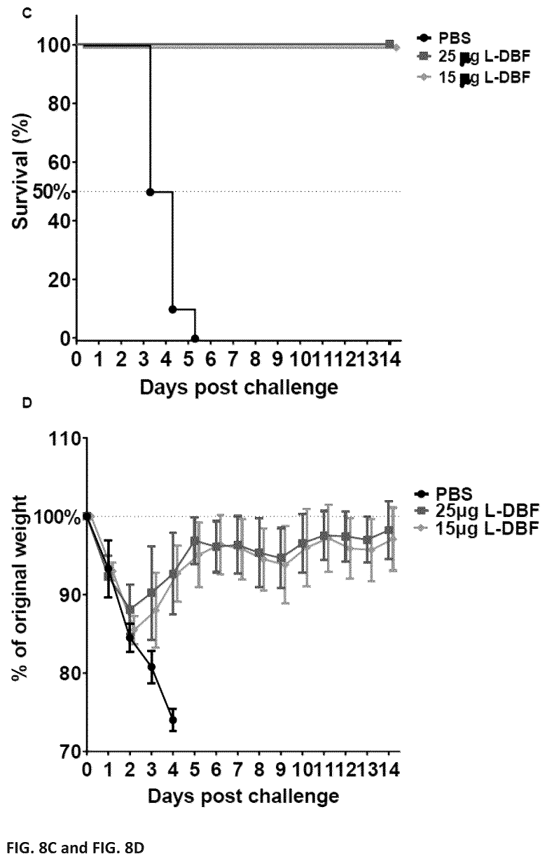

To characterize the dose response of L-DBF, we performed a dose escalation study by vaccinating mice IN with 1, 10, 15, or 25 μg of L-DBF. The resulting anti-IpaB and anti-IpaD serum IgG titers from the 10, 15 and 25 μg L-DBF doses were similar (FIGS. 2A, B—solid blue, yellow and red lines), while the titers for the 1 μg L-DBF dose were about one log unit lower (FIGS. 2A, B, solid green line). The anti-IpaB and anti-IpaD fecal IgA titers of 15 and 25 μg L-DBF doses groups were higher than 1 and 10 μg L-DBF doses groups on Day 42 and Day 55 (FIGS. 2A, B, dashed lines). Two challenge experiments were then performed. In the first, mice vaccinated IN with 1, 10, or 25 μg L-DBF were challenged with 1×10⁷ CFU/mouse of S. flexneri 2a. The 25 μg L-DBF dose elicited 80% protection, while the 1 and 10 μg doses were not protective (1/10 mice was protected) (FIG. 8A). Because the high challenge dose resulted in some death within the mice receiving the highest vaccine dose, we performed a second challenge using a slightly lower challenge dose with mice vaccinated using 15 μg or 25 μg of L-DBF. In this case the mice were 100% protected from a challenge dose of 1.5×10⁶ CFU/mouse (FIG. 8C). It is noted that while some mice failed to be protected at the higher challenge dose, the overall protection was not statistically different that the 100% protection at the lower challenge dose (p=0.15). Likewise, while the 15 and 25 μg vaccine doses are somewhat high, it should be noted that the L-DBF is not yet formulated to maximize the host response to the vaccine. Formulation will be required prior to use in humans since monomeric subunit proteins are often not protective in humans. We have determined that formulation of L-PaF, the Pseudomonas aeruginosa homologue of L-DBF, reduces the required antigen by 10-fold or more.

(6) Lungs from Mice Vaccinated Intranasally with 15 and 25 μg L-DBF have Higher Levels of Cytokines Related to a Th17 Response

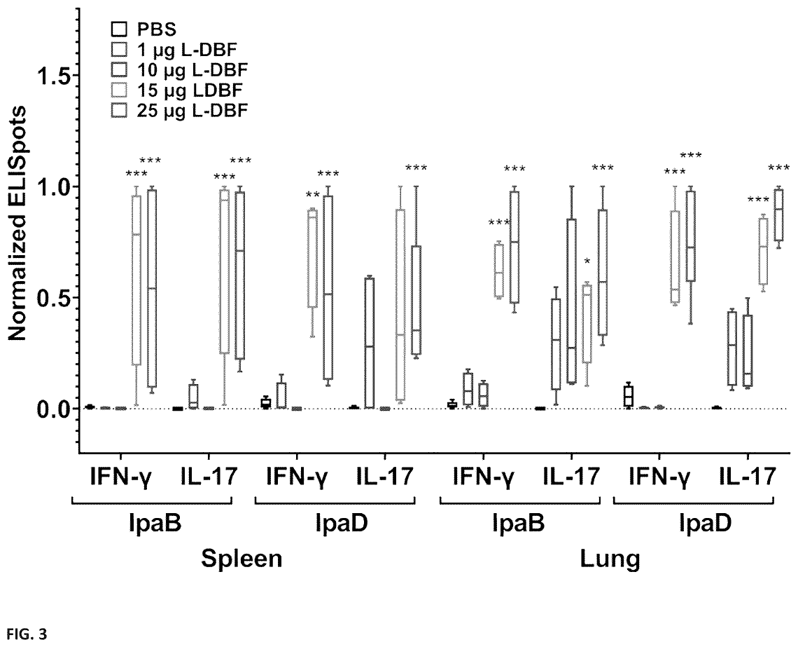

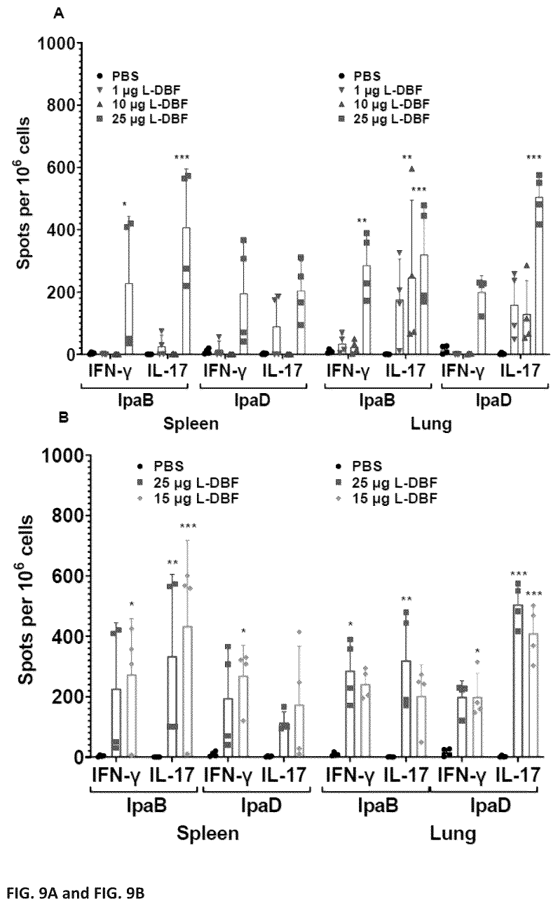

Having demonstrated the successful protective efficacy of 15 and 25 μg L-DBF doses and the failure of 1 and 10 μg doses, the lungs and spleens from the mice were examined for cellular immune responses. Cell suspensions from each organ were stimulated with IpaB or IpaD and the frequency of cells secreting IL-17A and IFN-γ cells enumerated by ELISpot analysis (FIG. 3 and FIG. 9). In both organs, mice vaccinated with 15 and 25 μg L-DBF generally have higher frequencies of IL-17a and IFN-γ secreting cells when stimulated with either IpaB or IpaD. Meanwhile the 1 and 10 μg doses do not generate secreting cell frequencies significantly higher than PBS.

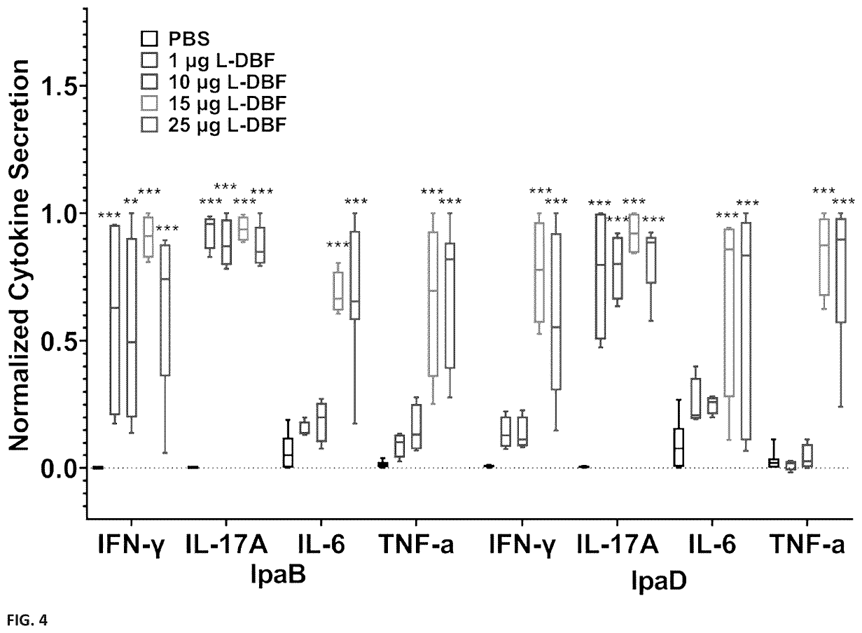

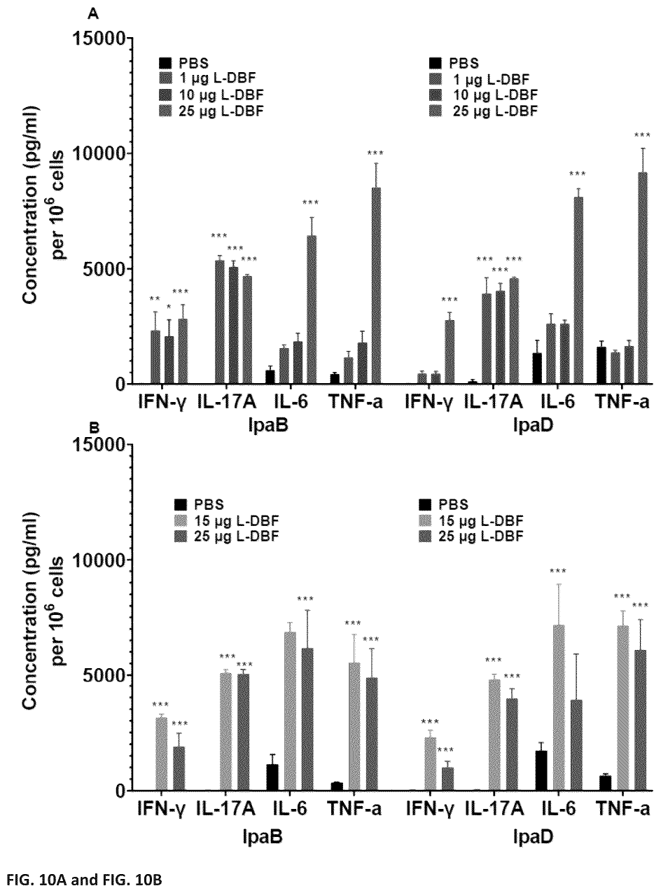

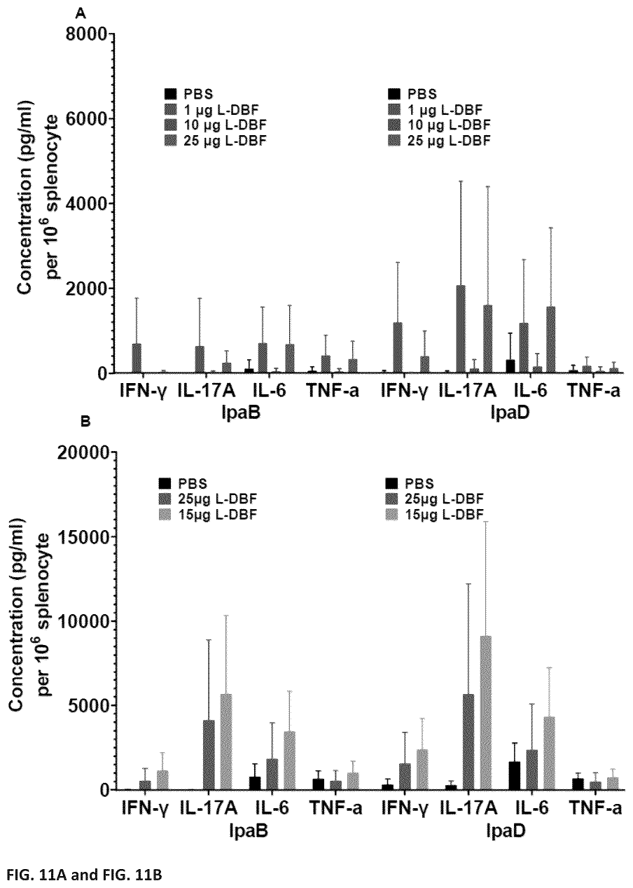

Using these same lung cells, we quantified secreted cytokine levels after stimulation with IpaB and IpaD (FIG. 4 and FIG. 10). When compared to the PBS vaccinated group, significantly higher levels of IL-17A were secreted from lung cells from all four L-DBF vaccinated groups after stimulation with IpaB or IpaD. In contrast, IFN-γ secretion from lung cells was significantly higher in all four L-DBF groups as compared to PBS groups when stimulated with IpaB, but only in the 15 and 25 μg L-DBF vaccinated group after stimulation with IpaD. When examining other cytokines, only the 15 and 25 μg L-DBF vaccinated group elicited significantly higher IL-6 and TNF-α secretion after stimulation with IpaB or IpaD. No statistical difference was seen in secretion of these cytokines from splenocytes (FIG. 11).

### c) Discussion

Diarrheal diseases are a severe global health problem. Shigellosis, in particular, is often lethal to children under five years of age, especially those living in developing countries where access to basic life-saving treatments and hygienic resources are limited. Although the morbidity and mortality of shigellosis have diminished in recent years, the emergence of antibiotic resistance among the shigellae, which can cause an infection with as few as 200 organisms, calls for an effective vaccine against shigellosis. Because target populations for vaccination reside in low and middle-income countries, a major concern in Shigella vaccine development is cost, which is negatively impacted by the need for a cold chain and the lack of public health resources. Broadly protective vaccines with simplified formulations and storage condition could meet the low-cost requirement.

In this study, we genetically fused the LTA1 subunit of dmLT with DBF. We found L-DBF provided comparable protection as DBF+dmLT when delivered IN but not when delivered via IM or ID routes. Vaccine efficacy depends on the route of administration. Before choosing the IN route, other routes, such as, ID, IM were tested with limited to no success, which was consistent with previous work and unpublished results). Transport of the administered immunogen to the local lymph nodes are an important determinant towards generation of a strong humoral and cellular response. Previous studies showed IM injected immunogens to be transported to the local nodes and is not disseminated systematically. In case of mice, IM injected immunogens are generally processed in the subiliac and popliteal lymph nodes, which are far from mucosal sites. IN route, on the other hand, showed significantly better response due to the presence of a strong mucosal immune response against the immunogen.

Additionally, L-DBF provides the much-desired cross-protection that is lacking with traditional LPS based vaccines including whole killed, live-attenuated and O-antigen based vaccines that are considered serotype specific. In contrast, the T3SS proteins IpaB and IpaD are conserved among all serotypes and have been considered attractive target antigens in subunit vaccine development. Early studies showed that DBF with dmLT administered IN protected mice against S. flexneri, S. sonnei and S. dysenteriae. In this study, intranasal L-DBF vaccinated mice elicited effective protection against five Shigella spp. subtypes, S. flexneri 2a, S. flexneri 3a, S. flexneri 6, S. flexneri 1b, and S. sonnei 53G.

The increase in the frequency of IL-17A and IFN-γ secreting cells, as well as the secretion of these cytokines by lung cells from mice vaccinated with 15 and 25 μg L-DBF, suggests that IL-17 and IFN-γ responses are required for protection. Additionally, the lack of an IFN-γ response after stimulation of lung cells from the 1 and 10 μg vaccinated mice, coupled with the reduced protection at these doses, points to the importance of the IFN-γ response. While we have previously published that vaccine formulations containing IpaD and IpaB trigger IL-17A and IFN-γ responses, we have not shown that the absence of such a response shows a parallel lack of protection. Furthermore, mice vaccinated with 15 and 25 μg L-DBF, which showed maximum protection against an S. flexneri challenge, elicited higher secretion of IL-6 and TNF-α. Pro-inflammatory mediators like TNF-α and IL-1β stimulate the expression of IL-6. Recent studies showed that sIgA could induce IL-6 by normal human lung fibroblasts (NHLFs). Moreover, sIgA has been found that could enhance Shigella taken up by M cells, which reduced the invasion of pathogens. Our results also detected higher titers of fecal IgA in mice vaccinated with 15 and 25 μg L-DBF, suggested a correlation between IgA and the IL-6 secretion. Further work will be required to demonstrate a correlation of protection of these cytokines.

A Th17 response is considered essential in the host immune defense against pathogens that target mucosal surfaces. Early studies suggested that a Th17 response was important for host survival in a lethal Shigella challenge. Therefore, a vaccine that induces a mucosal Th17 response would be expected to increase its efficacy. Our lab has shown that intranasal DBF with the adjuvant dmLT induced significantly higher IL-17A in mouse models, which is associated with successful protection against lethal challenge. Because DBF alone does not induce IL-17 when administered IN, dmLT must be responsible for IL-17 stimulation. Similar outcomes were found in this study for L-DBF. The results shown here also suggest there is a dose-dependent increase in IFN-γ production in L-DBF vaccinated groups. Research has shown that Th1 plays a significant role in protection of mice after Shigella challenge, with IFN-γ especially important for clearing intracellular Shigella via macrophage activation, which restricts intracellular growth of the pathogen. Stimulation of high levels of both IL-17 and IFN-γ, as well as the Th17 and Th1 related cytokines IL-6 and TNF-α, demonstrate that L-DBF mainly elicits Th1 and Th17 responses to protect mice against S. flexneri infection. In this study, we did not detect IL-4 or IL-5 (data not shown), which are related to the Th2 differentiation and response.

These data show that the adjuvant-antigen conjugate L-DBF is a viable candidate for a broadly protective Shigella vaccine. Intranasal immunization with L-DBF induces strong anti-IpaB and anti-IpaD IgG responses, as well as significant Th1 and Th17 response in lung, yielding effective protection against lethal pulmonary challenges with five Shigella spp. strains. Furthermore, the single protein nature of the LTA1-DBF conjugate simplifies and reduces the cost of vaccine production and formulation. While the mouse pulmonary model is not ideal, it is accepted in the field. It is a simplified model to study pathobiology of human adapted Shigella spp. Like the intestine, the lung is a lymphoid organ with antigen presenting cells, T helper and suppressor cells and B cells. In addition, the bronchus constitutes a mucosal surface similar to the intestinal mucosa with occasional lymphoid follicles like Peyer's Patches (PPs).

### Example 4: The L-DBF Vaccine Cross Protects Mice Against Different Shigella Serotypes after Prior Exposure to the Pathogen

Shigellosis is a severe gastrointestinal disease that is estimated annually to affect 90 million people globally with 164,000 deaths reported each year. Shigellosis disproportionately affects low-income regions of the world where potable water and proper sanitation are lacking. Children are particularly vulnerable, with mortality and morbidity rates highest among those under the age of five. Survivors often suffer from impaired growth due to malnutrition, which is exacerbated by repeated episodes of infection. Shigella spp. also cause diarrhea in travelers and military personnel in countries with low, middle incomes (LMIC) or even high incomes. The Shigella species includes S. flexneri, S. sonnei, S. dysenteriae, and S. boydii, which are further divided into more than 50 serotypes that offer little or no cross-protective immunity. S. flexneri is the primary species of endemic diarrhea in developing regions where there is limited access to a hygienic resource, whereas S. sonnei is the primary serotype in more developed regions. The symptoms of shigellosis include watery diarrhea, bloody dysentery, fever, intestinal cramps, and vomiting. Despite significant reductions in frequency due to improved sanitation, the rise of antimicrobial resistance in Shigella prompts the development of a safe and effective vaccine against this pathogen. While there are several vaccine candidates currently being developed, no licensed vaccine is available yet.

Resembling a syringe and needle, the T3SS apparatus (T3SA) is an energized nanomachine, that is used by the pathogen to inject virulence effector proteins into host cells to manipulate the host cell machinery for the benefit of the bacterium. The Shigella T3SA tip protein IpaD localizes to the T3SA needle tip and is required for secretion control and virulence. IpaD recognizes extracellular signals to trigger the surface localization of IpaB, the first Shigella T3SA translocator protein, to dock with IpaD at the needle tip. Because IpaD and IpaB are highly conserved among Shigella species, broadly protective subunit vaccines against Shigella can be developed using these proteins.

The intranasal (IN) administration of IpaD and IpaB with the mucosal adjuvant double-mutant heat labile toxin (dmLT) from enterotoxigenic E. coli provides protection in mice against challenge with both homologous and heterologous strains of S. flexneri and S. sonnei. By producing an IpaD-IpaB genetic fusion, called DBF, we were able to reduce production costs. When delivered IN, DBF admixed with dmLT induced immune responses similar to those stimulated by IpaD, IpaB, and dmLT. This formulation also provided cross-protection against S. flexneri, S. sonnei, and S. dysenteriae. Moreover, by genetically fusing LTA1, the active moiety of dmLT, to the N-terminus of DBF, we produced L-DBF and reduced the risk of the potential side effects of dmLT while further reducing production costs. L-DBF was found to elicit significant levels of IL-17 and IFN-γ in vaccinated mice, and was effective in protecting them against a lethal challenge of S. flexneri 2a. Importantly, L-DBF also conferred cross-protection against lethal heterologous challenges with S. flexneri 3a, 1b, 6 and S. sonnei. Both Th1 and Th17 responses are known to be important for protection against an S. flexneri infection.

The recent Global Enteric Multicenter Study (GEMS) found that 24% of shigellosis cases were caused by S. sonnei and 66% by S. flexneri in low-income countries while S. sonnei is responsible for a large majority of cases in middle- and high-income countries Thus, a broadly protective vaccine must cover not only a naïve population but also a population that has already experienced shigellosis. In this study, we examine the potential of L-DBF to elicit protective immunity in mice that have been pre-exposed to S. flexneri. We found that one or two sublethal exposures to S. flexneri 2a does not affect the subsequent immune responses in mice vaccinated with L-DBF. Mice pre-exposed to S. flexneri 2a produced bactericidal antibodies against S. flexneri 2a with the target of those antibodies being mainly LPS with a lower level of bacteriocidal anti-IpaB and anti-IpaD antibodies, however, these antibodies were not long lasting. In contrast, if the mice were vaccinated with L-DBF, the anti-IpaB and anti-IpaD antibodies were much more long lived. Moreover, mice pre-exposed with S. flexneri 2a were unable to survive a subsequent S. sonnei challenge, while pre-exposed mice subsequently vaccinated with L-DBF did survive S. sonnei challenge. Since it can protect against heterologous Shigella spp. serotypes, regardless of prior pathogen exposure, L-DBF has the can be a broadly protective vaccine against shigellosis in sporadic and endemic areas of the world.

### a) Results

(1) A Single Pre-Exposure to S. flexneri 2a is not Sufficient to Protect Mice from a Homologous Challenge while L-DBF does Protect Against Homologous or Heterologous Challenge.

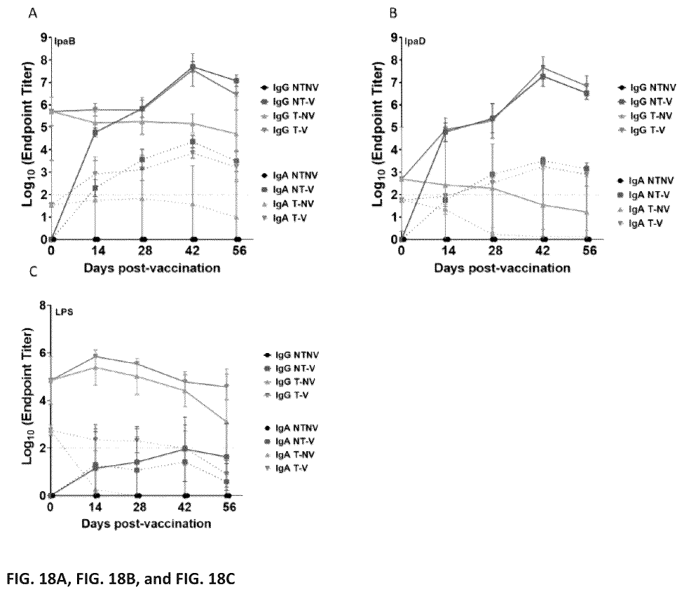

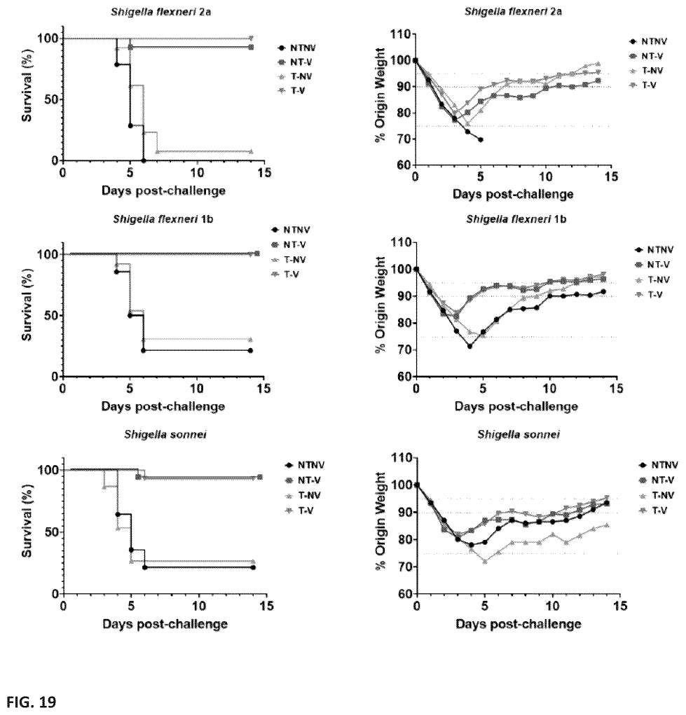

A single S. flexneri 2a exposure is not sufficient to elicit protective immunity in mice from a subsequent challenge, even against the homologous serotype. It's unclear, however, whether a prior infection can alter the protective immunity elicited by L-DBF. Therefore, we wanted to examine the immune response induced by L-DBF in naïve mice alongside those previously exposed to S. flexneri. Thus, half of a group of mice were pre-exposed with 6×10⁴ CFU of S. flexneri 2a on day-60. On day 0, half of the mice from the pre-exposed or “treatment” group (T) and half from the non-exposed or “no treatment” group (NT) were vaccinated (V) with 25 μg L-DBF IN (Table 7). The remaining mice from each group were administered PBS as negative controls (not vaccinated or NV). The kinetics of the antigen specific IgG and IgA response showed that all vaccinated mice had higher antibody titers than unvaccinated mice (FIG. 18). Pre-exposed mice (T) exhibited low antibody titers at the end of the pre-exposure period as compared to the high titers of anti-IpaD and -IpaB post-L-DBF vaccination (NT-V) (FIGS. 18A and 18B). However, anti-IpaB and -IpaD IgG and IgA titers increased to comparable levels after the pre-exposed mice were immunized with L-DBF (T-V). Very low titers of anti-LPS IgG in the pre-exposure groups were also observed, which decreased over time (FIG. 18C). Although some anti-LPS IgG and IgA were detected in NT-V, they are below our cut-off. These four groups of mice (NT-NV, NT-V. T-NV. T-V) were challenged on day 56 with a potentially lethal dose of S. flexneri 2a (6×10⁶ CFU), S. flexneri 1b (4×10⁶ CFU) or S. sonnei (1×10⁶ CFU). Fewer than 30% of the unvaccinated mice (NT-NV, T-NV) survived the challenges including those treated groups that were challenged with the homologous S. flexneri 2a strain. In contrast, >90% of those mice vaccinated with L-DBF (NT-V, T-V) survived the Shigella spp. challenge, regardless of challenge species. Table 7 demonstrates the broadly protective capacity of L-DBF. Similar results were seen in the weight loss curves (FIG. 19). Those mice that were vaccinated with L-DBF (NT-V and T-V) consistently demonstrated an earlier initiation of weight gain during recovery from the challenge than did the small number of unvaccinated mice (NT-NV and T-NV) that survived the challenge. We have found that weight gain is a strong early indicator of survival.

TABLE 7
Protective efficacy of L-DBF against Shigella
spp. serotypes following a single pre-exposure.

| | | Pre-exposure | | Homologous | | |
|---|---|---|---|---|---|---|
| | | treatment (T) | Vaccination (V) | challenge | Heterologous challenge | |
| Set | Grp | S. flexneri 2a | 25 μg L-DBF | S. flexneri 2a | S. flexneri 1b | S. sonnei 53G |
| A | 1 | NT | NV | 0% | — | — |
| | 2 | NT | NV | — | 20% | — |
| | 3 | NT | NV | — | — | 20% |
| B | 4 | NT | V | 90% | — | — |
| | 5 | NT | V | — | 100% | — |
| | 6 | NT | V | — | — | 90% |
| C | 7 | T | NV | 10% | — | — |
| | 8 | T | NV | — | 30% | — |
| | 9 | T | NV | — | — | 30% |
| D | 10 | T | V | 100% | — | — |
| | 11 | T | V | — | 100% | — |
| | 12 | T | V | — | — | 90% |

The pre-exposure groups were treated as follows: Sets A&B were not pre-exposed to S. flexneri 2a (not treated or NT). Sets C&D were pre-exposed to S. flexneri 2a (T). On day 0, sets A&C were administered PBS (not vaccinated or NV). Sets B&D were vaccinated with L-DBF (V). On day 56, all mice were challenged with results given as % survival.

(2) Two S. flexneri 2a Pre-Exposures does Protect Against a Subsequent Homologous Challenge and does not Affect the Homologous or Heterologous Protection Elicited by L-DBF.

While we felt it was important to show that pre-exposure to Shigella did not reduced L-DBF efficacy, it is equally important to shown that multiple pre-exposure episodes that do provide subsequent protective immunity against a given serotype do not adversely affect the L-DBF vaccination. A single Shigella infection does not protect against a subsequent homologous challenge, however, two such infections does provide protection against a third challenge by the same serotype. We therefore exposed mice to a lower dose (10⁵ bacteria), allowed them to fully recover and then exposed them to a second larger (6×10⁵) and allowed to again recover. We then vaccinated with L-DBF to determine the effect of the double exposure on the resulting immune response.

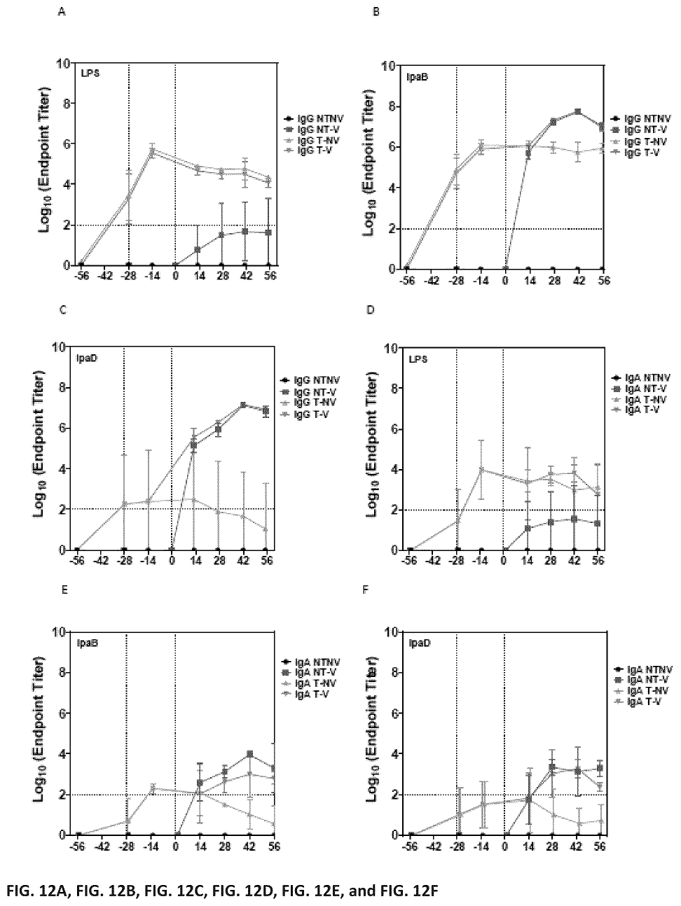

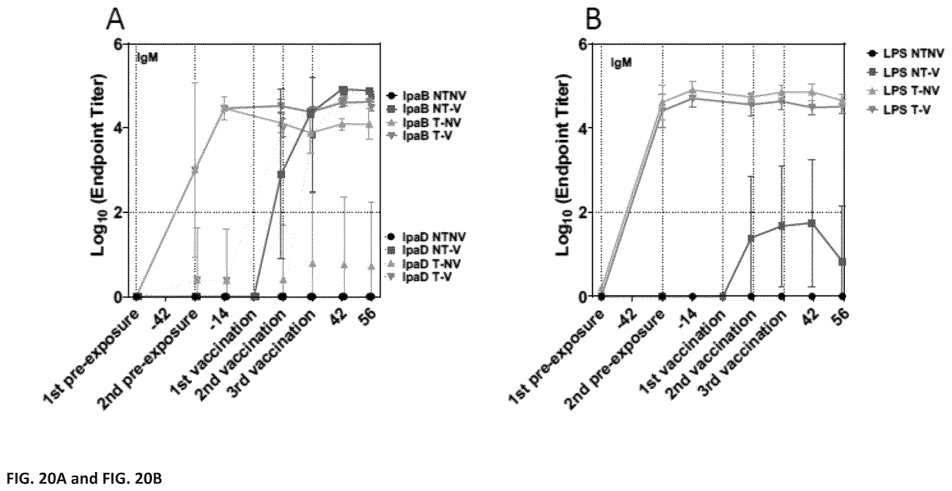

For these experiments, half of the mice were pre-exposed to S. flexneri 2a on days −56 and −28 with 82% of the mice surviving both pre-exposures. On day 0, half of the mice from the pre-exposed treated group (T) and half from the non-exposed or not treated group (NT) were vaccinated with 25 μg L-DBF IN (V). The remaining mice from each group were administered PBS to serve as not vaccinated controls (NV). As compared to a single pre-exposure, anti-LPS IgG titers after two-pre-exposures were approximately the same at day 0 (T-NV, T-V), however, the durability of the IgG response was better with a second pre-exposure (FIG. 12A and FIG. 18C). In contrast, while class switching (in terms of anti-LPS IgA titers) were considered at background levels (log=2) after a single pre-exposure, they were significantly higher than background levels after two pre-exposures. Furthermore, these titers were durable since they were maintained above background at day 56 (FIG. 12A). All mice vaccinated with L-DBF elicited higher antibody titers than non-vaccinated mice (FIG. 12). The maximal anti-IpaB IgG titers were comparable regardless of number of pre-exposures (FIG. 12B and FIG. 18A). Similarly, after the prime vaccination (as detected in day 14 samples), T-NV and T-V mice elicited comparable anti-IpaB IgG, IgA and IgM titers to those of NT-V (FIG. 12B, FIG. 20A) with the anti-IpaB IgM titers of the NT-V being slightly lower. After the first boost on day 14, the anti-IpaB IgG and IgA titers declined in the T-NV group but showed an increase in the T-V group compared to the high titers seen for the NT-V mice. Thus, anti-IpaB antibody responses were similarly primed regardless of the number of pre-exposures, albeit the response was faster upon vaccination for the mice with two pre-exposures, reaching maximal titers after the first boost on day 14 rather than with the second boost at day 28 as seen for the single pre-exposure mice. While the anti-IpaB and anti-IpaD kinetics of IgG and IgA titers were similar after a single pre-exposure, the anti-IpaD IgG and IgA kinetics after the two dose S. flexneri 2a pre-exposure were distinct (FIG. 12C and FIG. 20A). Anti-IpaD IgM, IgG and IgA were considered below baseline for all four groups prior to vaccination (day-14) (FIG. 12C and FIG. 20A). In contrast, at day 14 the T-V group had a significant increase in anti-IpaD IgM and IgG and an increase above baseline for IgA while the T-NV mice remained at pre-vaccination levels and continued to decrease thereafter. Notably, all mice tested showed anti-IpaB antibodies, but only 60% of serum from the T-NV mice contained detectable anti-IpaD antibodies, which led to the large standard deviations. This can be due to the difference in immunogenicity between these two subunits. Based on the anti-IpaD antibodies levels elicited after the prime and first boost for the NT-V mice, it appears that the second pre-exposure primed the T-V immune response to IpaD and the vaccination at day 0 provided a boost that increased antibody titers. Maximal titers for both NT-V and T-V groups were reached after the third L-DBF vaccination.

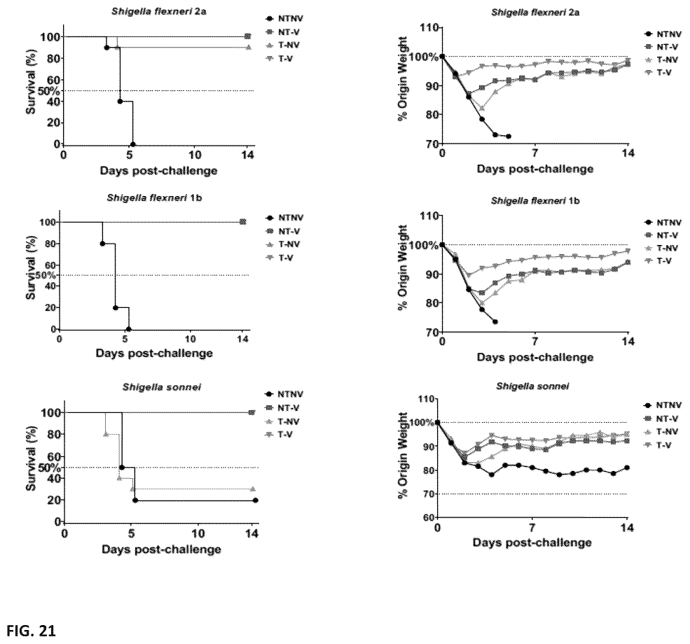

The four groups of mice were challenged on day 56 with lethal doses of S. flexneri 2a (1.5×10⁶ CFU/mouse), S. flexneri 1b (1.5×10⁶ CFU/mouse), or S. sonnei (1×10⁶ CFU/mouse) (Table 8). The two-dose pre-exposure of S. flexneri 2a provided >90% protection against the homologous o S. flexneri 2a challenge and the S. flexneri 1b challenge, which is in the same serogroup as 2a. In contrast, the protection afforded by S. flexneri 2a was only 30% when challenged with S. sonnei. Most importantly, 100% of the mice from all groups vaccinated with L-DBF survived and <20% of the mice from the NT-NV group survived (Table 8). Similar results were seen when weight loss was considered (FIG. 21). As expected, mice from T-V groups showed better weight recovery following homologous challenge (2a or 1b) than the NT-V or the T-NV. In contrast, the T-V and NT-V essentially showed equivalent weight gains, which were better than the T-NV group.

TABLE 8
Protective efficacy of L-DBF against Shigella
spp. serotypes following two pre-exposures.

| | | Pre-exposure | | Homologous | | |
|---|---|---|---|---|---|---|
| | | infection | Vaccination | challenge | Heterologous challenge | |
| Set | Grp | S. flexneri 2a | 25 μg L-DBF | S. flexneri 2a | S. flexneri 1b | S. sonnei 53G |
| A | 1 | NT | NV | 0% | — | — |
| | 2 | NT | NV | — | 0% | — |
| | 3 | NT | NV | — | — | 20% |
| B | 4 | NT | V | 100% | — | — |
| | 5 | NT | V | — | 100% | — |
| | 6 | NT | V | — | — | 100% |
| C | 7 | T | NV | 100% | — | — |
| | 8 | T | NV | — | 90% | — |
| | 9 | T | NV | — | — | 30% |
| D | 10 | T | V | 100% | — | — |
| | 11 | T | V | — | 100% | — |
| | 12 | T | V | — | — | 100% |

The double pre-exposure groups were treated as follows: Sets A&B were not pre-exposed to S. flexneri 2a (NT). Sets C&D were pre-exposed to S. flexneri 2a (T). On day 0, sets A&C were administered PBS (NV). Sets B&D were vaccinated with L-DBF (V), V. On day 56, all mice were challenged with shown given as % survival.

(3) S. flexneri 2a Pre-Exposure Elicits High Levels of Bactericidal Antibodies that can be Blocked by LPS, but not by IpaB or IpaD.

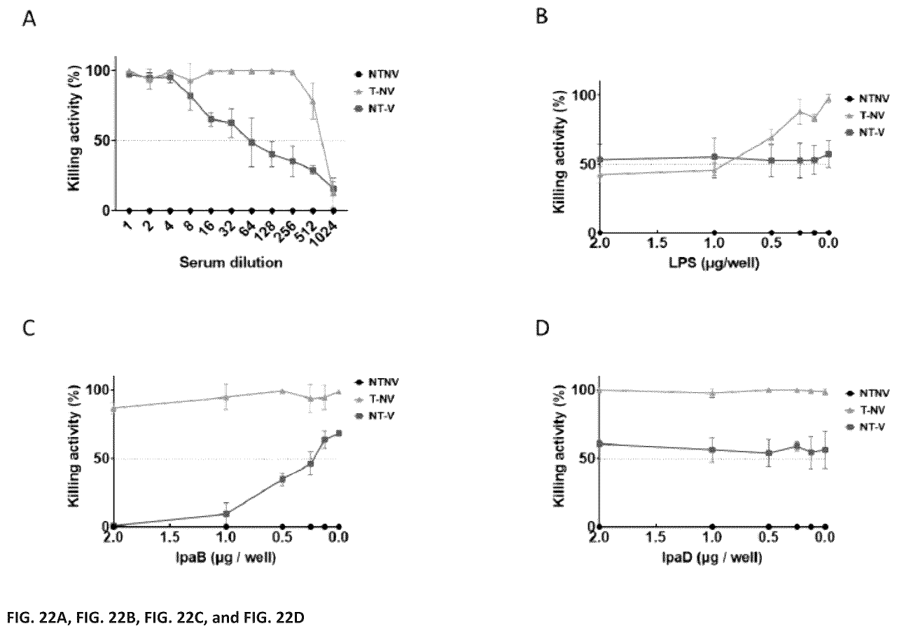

To determine whether the sera from mouse groups treated once with S. flexneri 2a or vaccinated with L-DBF elicited serum bactericidal activity (SBA), we tested the killing activity of the serum immunoglobulins against S. flexneri 2a (presented here as killing index or KI). Sera from both T-NV and NT-V mouse groups with one pre-exposure showed SBA while sera from the NT-NV did not kill bacteria (0%; as baseline) (FIG. 22A). Antibodies from the serum (1:512 dilution) of the T-NV mouse group exhibited >60% bactericidal activity (SBA KI=1163.11; log=3.07; p<0.001), while antibodies from the serum of the NT-V mouse group (1:64 dilution) showed an increase in the killing activity around 50% (SBA KI=59.84; log=1.78, p<0.001). We propose that the killing activity present in the sera from pre-exposed mouse groups (T) can be attributed to anti-LPS antibodies, while the activity present in the L-DBF vaccinated mice (V) can be attributed to anti-IpaD and -IpaB antibodies. To test this theory, we added IpaD, IpaB or LPS to the SBA mixture to block SBA activity (FIGS. 22B, 22C, and 22D). Indeed, the bactericidal antibodies in the serum from T-NV were blocked by LPS, but not by IpaB or IpaD (p<0.05) when comparing the outcomes of SBA from 2 μg, 1 μg or 0.5 μg LPS dilution point to those from higher dilution points (FIGS. 22B, 22C, and 22D). As expected, LPS did not affect the killing activity of the serum from the NT-V group (p>0.999 among all LPS dilution points). It should be noted here that the sera from the NT-V was used at 1:64 since a lower dilution, which had higher SBA activity, would have consumed the available pooled serum quickly, thereby preventing completion of these studies. Nevertheless, while the SBA activity of the 1:64 dilution of NT-V serum was 50%, it remained at 50% regardless of the amount of LPS used in the competition assay. IpaB, but not IpaD, blocked the SBA antibodies in the NT-V mouse group, with p<0.05 when comparing the outcomes of SBA from 2 μg, 1 μg, 0.5 μg or 0.25 μg IpaB dilution point to those from higher dilution points (FIGS. 22C and 22D). These results indicate that, though antibodies in the sera from both T-NV and NT-V mouse groups showed high SBA activity, such activity can be attributed to distinct antigens. Although having a higher SBA KI (log 10˜ 3), the high SBA antibody levels elicited from a single pre-exposure did not elicit protection against lethal Shigella challenges.

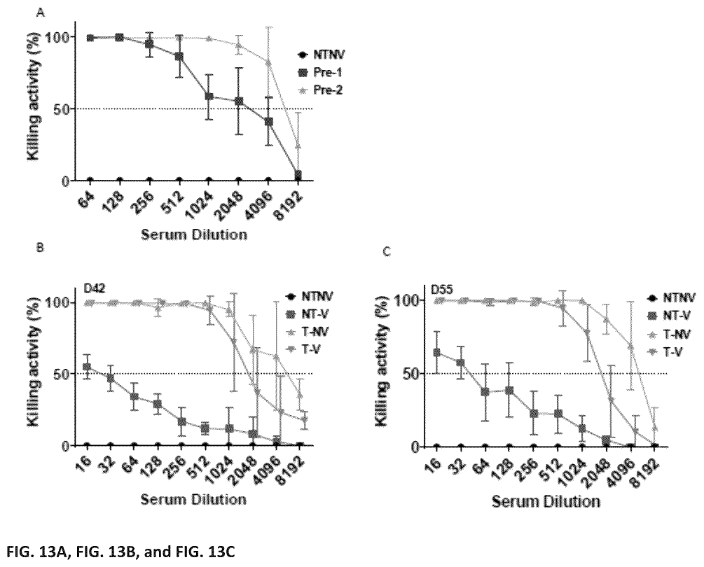

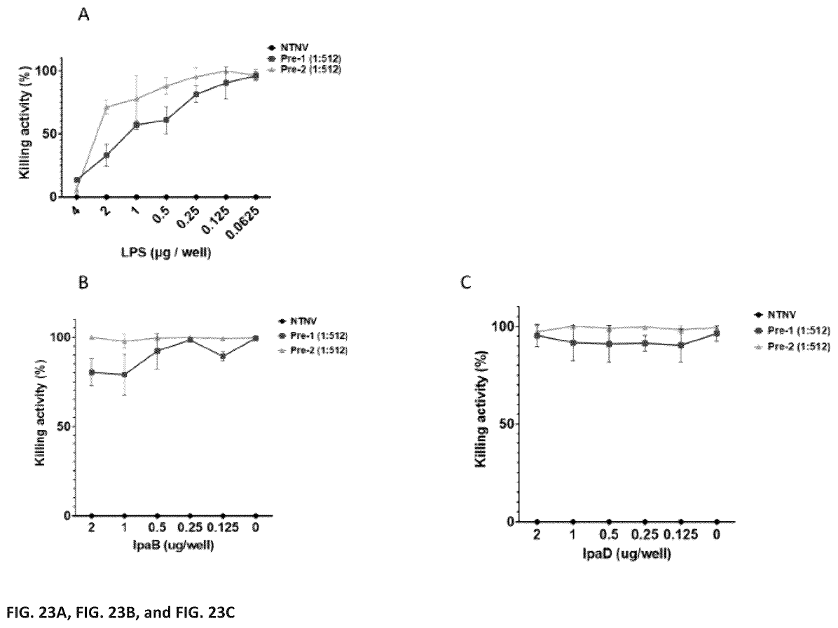

After the first pre-exposure, mouse serum showed a KI of 1116.68 (log=3.05), while after the second exposure, the KI was increased to 10753.53 (log=4.03) (FIG. 13A). The difference in the SBA between the first and second pre-exposures was statistically observed after a 1:1024 serum dilution (p<0.01). The SBA activity for sera from mice after the first and second exposures were not affected by the addition of IpaB or IpaD but was by purified LPS (p<0.05) when comparing the outcomes of SBA from lower LPS dilution points to those from higher dilution points (FIG. 23). Regardless of group, when the serum collected on day 42 (14 days after the second booster) is compared with that collected on day 55 (27 days after the second booster), SBA activity for the NT-V group was not significantly affected over the course of the L-DBF vaccination period (at 1:4096 dilution, killing activity dropped from 2.8% to 0%) (FIGS. 13B and 13C). In contrast, the SBA KI was reduced in the T-V group (SBA KI=2629.48-2920.9; log=3.41-3.46) compared with T-NV (SBA KI=11858.07-13812.4; log=4.07-4.14) on day 42 and 55. This is especially true on day 55 since after the 1:1024 serum dilution, the SBA of the serum from T-V group was significantly lower than for T-NV (on day 42, P values of T-NV vs. T-V were <0.15 at a 1:1024 serum dilution, and p=0.032 at 1:8192). This change may have been caused by the reduction in the number of LPS targeting bactericidal antibodies. When comparing the 1:8192 serum dilution between day 42 and 55, killing activity of T-NV dropped from 35% to 13.5% (p<0.001 on day 55 at 1:8192 compared to NT-NV), while the killing activity of T-V dropped from 18% to 0% (p>0.999 on day 55 at 1:8192 compared to NT-NV). Although SBA KI, which is based upon a midpoint value, did not show a significant change between day 42 and 55, the drop on killing activity at the end of dilution point indicated the SBA activity was reduced once stopping the pre-exposure or vaccination. Thus, bacterial clearance by this mechanism was significantly less effective.

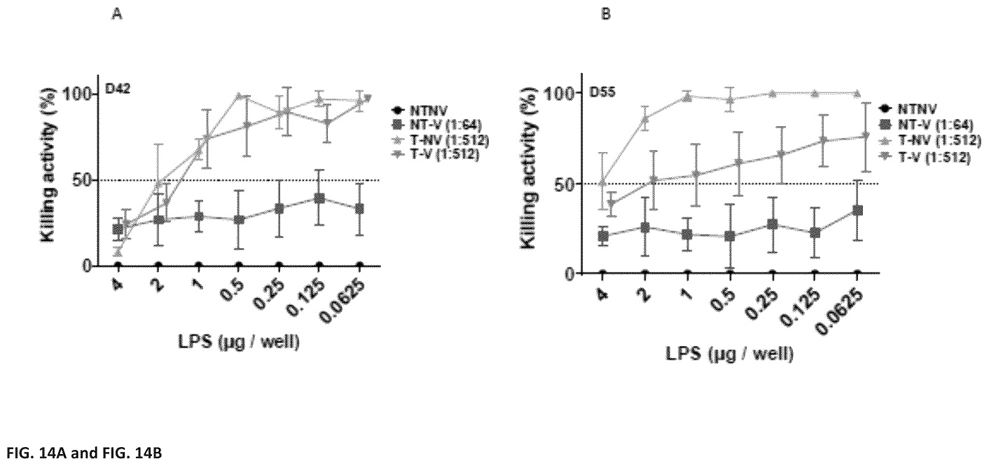

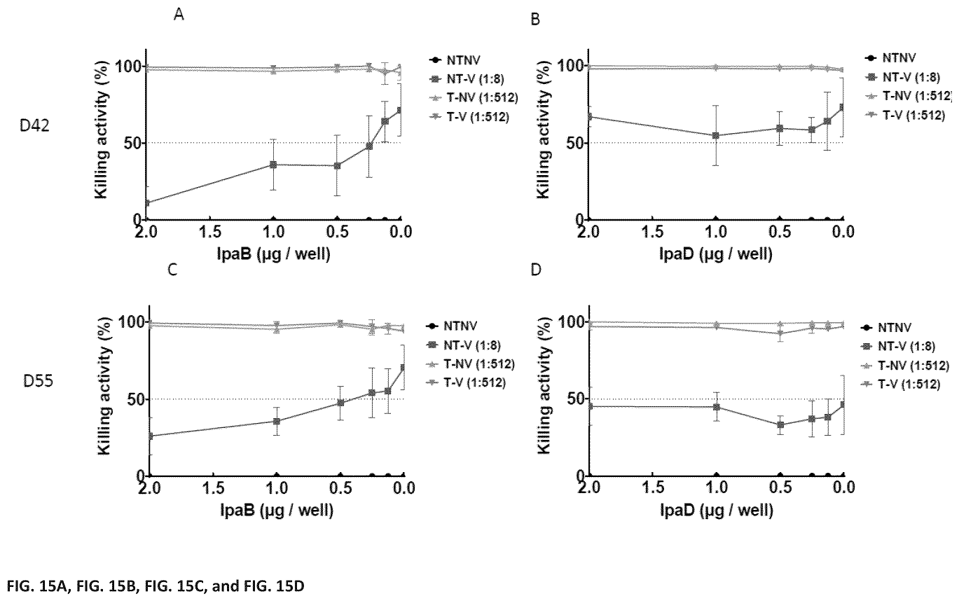

We next assessed the killing activity of the different pooled sera by using S. flexneri 2a LPS as a competitor for the bactericidal function (FIG. 14). SBA activity from NT-V mice was not affected by the addition of LPS with no significant difference seen at the different LPS concentrations. In contrast, the SBA for the T-NV and T-V mice can be reduced by competition with LPS in a dose-dependent manner (p<0.05) as shown by comparing the SBA results using 4 μg, 2 μg or 1 μg LPS concentrations to the more diluted LPS concentrations. Serum from T-V group only had 75.8% killing ability with 0.625 μg LPS whereas the SBA from T-NV mice was not affected by the same amount of LPS on day 55 (p=0.002). Meanwhile, the SBA for the NT-V mice mainly targeted the IpaB rather than IpaD since the SBA activity can be readily inhibited in a concentration dependent manner by adding IpaB. The killing activity for NT-V pooled serum in the presence of 2 μg or 1 μg IpaB was significantly lower than that for the without IpaB added on day 42 or 55 (p<0.05). This was not the case for the addition of IpaD (p>0.1 among different IpaD dilution points on day 42 or 55) (FIG. 15). These results indicate that while SBA in both pre-exposed mouse and L-DBF vaccinated mouse groups showed high bactericidal activity, such activity can be attributed to distinct antigens. With respect to the mice exposed once versus twice with S. flexneri 2a, the SBA KI attributable to anti-LPS antibodies indicates that an SBA KI of 103 (seen for the single exposure mice) was not sufficient to protect mice from a Shigella lethal challenge. Rather, it appears that an SBA KI of potentially >10⁴ (seen for the double exposure mice) may need to be attained to protect the mice against homologous Shigella challenge without the support of vaccination with L-DBF.

(4) Shigella Pre-Infection Tends to Elicit IL-6 while Vaccination with L-DBF Favors Induction of IFN-γ and IL-17.

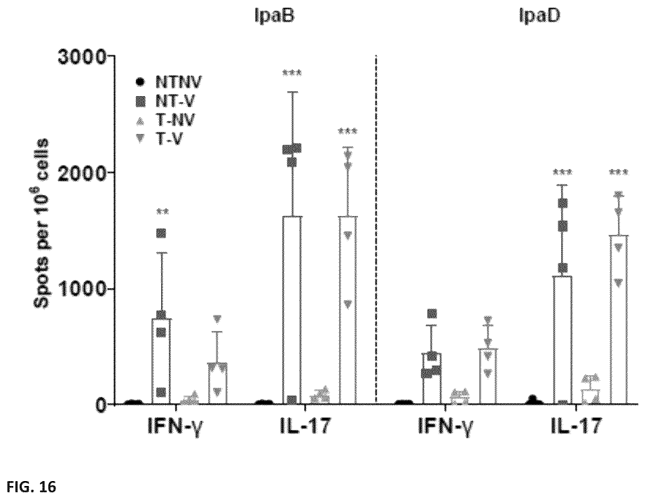

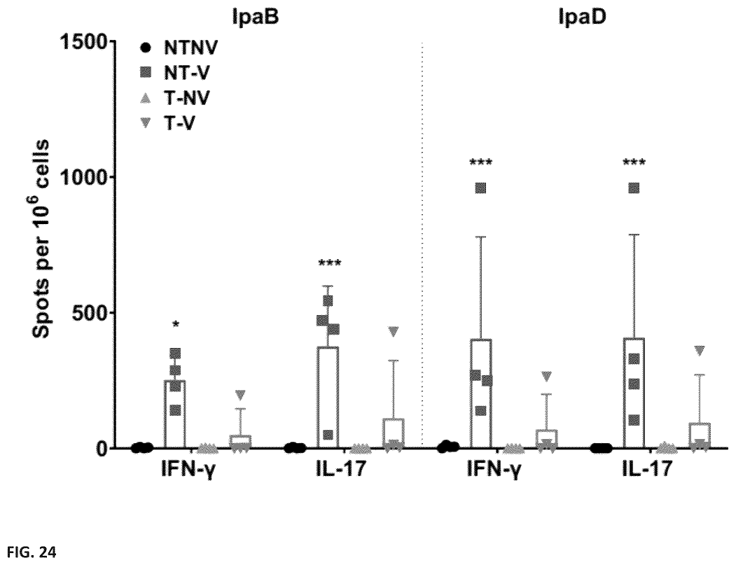

To assess why vaccinated mice from NT-V groups have a relatively lower SBA KI (<100) but are still able to be protected from lethal challenge from homologous and heterologous Shigella serotypes, we harvested lungs and spleens from all four groups prior to challenge to assess their immune responses. Cell suspensions from each organ were stimulated with IpaB or IpaD and the frequency of IL-17 and IFN-γ secreting cells were enumerated by ELISpot (FIG. 16 and FIG. 24). The L-DBF vaccinated mice exhibited comparably higher frequencies of IL-17 secreting cells in lungs when compared to the NT-NV mice, regardless of pre-exposure status. Likewise, frequencies of IFN-γ secreting cells in vaccinated mice were visibly higher when compared to NT-NV mice (IpaD IFN-γ secreting cells of NT-V vs. NT-NV: p=0.174; IpaB IFN-γ secreting cells of T-V vs. NT-NV: p=0.306; IpaD IFN-γ secreting cells of T-V vs. NTNV: p=0.122). NT-V mice induced higher IFN-γ and IL-17 secreting cells in spleen while T-V mice did not (FIG. 24). This is despite the fact that all the pre-exposed mice exhibited strong antibody responses against both proteins and IpaB in particular.

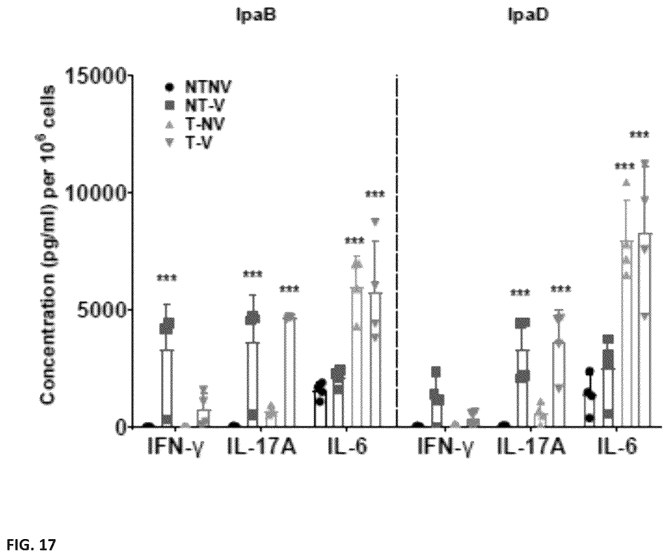

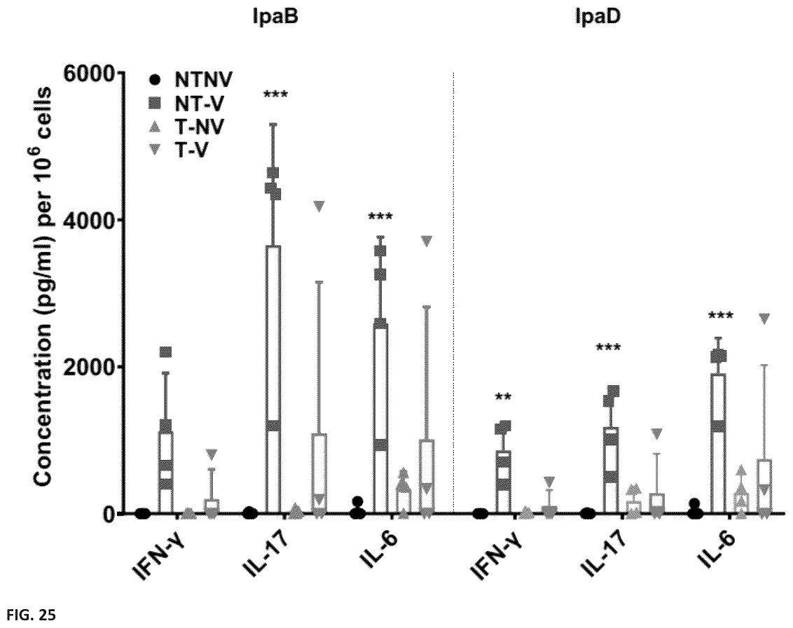

We quantified overall cytokine levels secreted from these cells following stimulation with IpaB or IpaD (FIG. 17 and FIG. 25). Higher levels of IFN-γ and IL-17A were secreted from lung cells from NT-V groups after stimulation with IpaB or IpaD than the NT-NV groups (IpaD IFN-γ of NT-V vs. NT-NV: p=0.085). Lung cells from T-V groups secreted significantly higher levels of IL-17A and IL-6, but not IFN-γ (IpaB: p=0.124; IpaD: p=0.105). Pre-exposure mice displayed higher IL-6 relative to the NT groups (FIG. 17). For the spleen cells, only the NT-V mice exhibited higher levels of IFN-γ (IpaB: p=0.067; IpaD: p=0.029), IL-17 (IpaB: p=0.021; IpaD: p=0.022) and IL-6 (IpaB: p=0.022; IpaD: p=0.004). (FIG. 25). These cytokine responses indicate that there are differences in the nature of the immune responses induced in mice by pre-exposure to Shigella versus those vaccinated with L-DBF. The IL-17 responses to these proteins by mouse lungs was dependent upon L-DBF vaccination and was not noticeably affected by the pre-existing immune response from pre-exposure. It is this response that leads to serotype-independent cross-protection against lethal Shigella challenge.

### b) Discussion

Shigellosis is an important public health issue worldwide. Shigella infections can have high morbidity and mortality among children under five years old, especially those living in developing countries where access to basic life-saving treatments and hygienic resources are limited. Although the mortality of shigellosis is on the decline, morbidity remains high and antibiotic resistance is rapidly emerging, which calls for a broad, economic, and effective vaccine against shigellosis. Since most of the target population for a Shigella vaccine reside in low-middle income countries (LMICs), major concerns that must be considered in developing a vaccine are the production costs, the need (or the lack thereof) for a reliable cold chain, and the lack of public health resources. These factors make it difficult for the LMICs to acquire the stockpile needed to secure adequate dosage for their countries. Thus, safe vaccines in simple formulations, offering broad-spectrum protection are required to ensure cost effectiveness, as well as their efficient use.

L-DBF induces protective immune responses against five unique Shigella spp. serotypes with the protective response appearing to correlate with the levels of secreted IL-17 and IFN-γ at the site of infection. A drawback of this prior work, however, is the absence of understanding of the immunity against Shigella spp. in an endemically infected population. Most LMICs are endemic for shigellosis and a dampened immune response in pre-exposed population would lessen the impact of an L-DBF subunit vaccine. While we realize there are certain limitations in mice for this study, we assessed the immune response in mice previously exposed to S. flexneri 2a and found that pre-exposure did not dampen the immune response or lessen the efficacy against Shigella spp. in L-DBF vaccinated mice. Moreover, we continue to see an interesting interplay between Th17 and Th1 in the form of IL-17A and IFN-γ, respectively, in conjunction with IL-6.

Pre-exposure with one sublethal dose of S. flexneri 2a failed to protect mice from subsequent lethal challenge from homologous or heterologous strains. While not protective against subsequent lethal challenge, a single pre-exposure did give rise to antibodies capable of bactericidal activity. The serum bactericidal assay is a complement fixation assay driven by antibodies present in serum that allow for the formation of membrane attack complex (MAC). While this is an important determinant in the humoral arm of immune response, these findings indicate that there is a strong cellular response needed to neutralize intracellular bacteria like Shigella. Pre-exposure can elicit a strong humoral response, but it appears to fail at generating the appropriate strong T cell response needed for full protection.

In contrast to a single exposure, pre-exposure with two sublethal doses of S. flexneri 2a protected the mice from homologous but not heterologous challenge. These results are in line with the original mouse lung model publication where it was demonstrated that two doses of sublethal infection were required to induce 56-79% protection from a subsequent lethal challenge. The literature indicates antibodies are an important determinant for protection against infection by Shigella spp., so we examined the generation of bactericidal antibodies in pre-exposed mice. The results revealed a log-fold increase in the SBA titers for LPS in mice subjected to two pre-exposures as compared to mice subjected to a single pre-exposure. Further SBA studies along with ELISAs showed the SBA activity was limited to an increasing anti-LPS response following pre-exposure. This increase indicates a role of B cell epitopes in mounting a protective response against Shigella spp., which was found to be limited to homologous challenge only. Although effective, these T-cell independent responses neither generated long-term protection nor did they protect mice from a heterologous challenge. These results indicate the need for a vaccine with a long-lasting memory response and a protein vaccine that elicits a T-cell dependent response.

Generation of protective immune responses against Shigella spp. depends on both B and T cell response, which require the appropriate cytokine response. A large spike in lung IL-6 was observed in pre-exposed mice which was absent in the vaccinated mice, while a balanced Th1/Th17 response was not detected in the pre-exposed mice. Since the mouse pre-exposure with a sublethal dose of S. flexneri 2a should be representative of a naturally acquired infection, any unique responses in these mice should be considered as an expected outcome of the vaccine in humans in endemic regions. It has been indicated that natural infections “prepare” the immune system to generate a robust antigen-specific, long-lived humoral immune memory, which is true for both viral and non-viral intracellular pathogens. Moreover, studies involving Salmonella enterica vaccine development showed that mice pre-exposed to Salmonella vaccine vectors, compromises their cell-mediated immunity, especially the CD8+ response. We did not see this in our study after vaccination with L-DBF despite the presence of high anti-LPS SBA antibodies in the serum of pre-exposed mice. Pre-exposure resulted in significantly higher IL-6 and lower IFN-γ in the lungs of mice, but there were no notable differences were seen for TNF-α, IL-12p70, and IL-1β. Although important for B cell priming and differentiation to plasma cells and induction of T cell responses, high IL-6 has been shown to be a major factor in derailing the immune system. Previous studies have demonstrated the ability of Shigella to impair human T lymphocyte responsiveness. It does so by compromising the CD4 T cell F-actin cytoskeleton dynamics resulting in cortical stiffness of the cell. Once infected, the scanning ability of these T cells upon contacting the APCs was found to be impaired, leading to decreased cell-cell contact, compared to the T cells in non-infected humans. Moreover, CD8 T cells fail to respond to other antigens, if presented with Shigella. The T cell unresponsiveness in Shigella-infected cases have been observed by other groups as well, where CD4 T cell motility in the lymph nodes were seen to be dropping drastically. The generation of the immune response is also impacted by the length of time between two successive doses and/or infections. Usually, IFN-γ and IL-6 work in tandem, but this was not seen in the present study, where pre-exposure led to a high IL-6 response, but lower IFN-γ secretion in the lung. Interestingly, pre-exposure did not lead to a dampened T cell response during the subsequent vaccination. We found both Th1 and Th17, along with IL-6, to be important to mount an anti-Shigella immunity. All these results indicate that although B cells are primed to generate an immediate short-term anti-Shigella immune response, the T cell mediated immune response is dampened following Shigella infection. The fact that pre-exposure dampens the immune response and makes it difficult to mount a protective response in already infected mice, indicates that a vaccine seems essential for long term protection.

Since the Th17 response is uninhibited following pre-exposure, L-DBF was able to generate a protective response in these mice. IL-17 is an important cytokine that protects the body from mucosal bacterial challenges. L-DBF vaccinated mice, whether previously exposed or not, generated significantly higher levels of IL-17A. Only the unexposed vaccinated group showed an elevated response of IFN-γ, and all the other groups failed. These results indicate the inability of the pre-exposed mice to generate a strong Th17, Th1 response. It also indicates these cell types are required for generating a protective immune response against Shigella in mice.

In conclusion, L-DBF is a self-adjuvanting subunit vaccine candidate that can induce a protective immune response against multiple Shigella serotypes and species, regardless of whether the host has had prior Shigella exposure or not. While other subunit vaccines have seen tested, the inclusion the two essential and surface exposed immunogens of IpaD and IpaB makes this vaccine platform unique. Then the addition LTA1 and its ability to elicit a strong mucosal immune response indicate that L-DBF is an attractive vaccine candidate for use in regions where shigellosis is endemic.

### c) Materials and Methods

### (1) Materials

Unless otherwise noted, all reagents were from Sigma or Thermo Fisher and were chemical grade or higher. S. flexneri 2a 2457T was provided by A. T. Maurelli, University of Florida, Gainesville, Fl. S. flexneri 1b, and S. sonnei 53G were provided by Eileen Barry, University of Maryland School of Medicine, Baltimore, MD. Shigella LPS was provided by R. K. Ernst (University of Maryland School of Dentistry).

### (2) Protein Production

Plasmids expressing IpaD, IpaB or L-DBF were used to transform E. coli Tuner (DE3) cells. The transformed bacteria were grown in a 10 L bioreactor. Protein expression was induced with 1 mM IPTG and the bacteria grown at 37° C. for an additional three hours. The bacteria were collected by centrifugation, resuspended in IMAC binding buffer (20 mM Tris-HCl pH 7.9, 500 mM NaCl, 10 mM imidazole) with 0.1 mM AEBSF and lysed using a microfluidizer at 18,000 psi with three passes. The cellular debris was removed by centrifugation and the clarified supernatant applied to a nickel-charged IMAC column. The His-tag IpaD (HT-IpaD) was then eluted with IMAC elution buffer (20 mM Tris-HCl pH 8.0, 500 mM NaCl, 500 mM imidazole). The HT-IpaD was applied to a Q column to remove residual LPS and dialyzed against PBS and stored at −80° C. IpaB and L-DBF were expressed with HT-IpgC, the cognate IpaB chaperone. The IpaB/HT IpgC or L-DBF/HT-IpgC was purified using the IMAC procedure as for HT-IpaD. The complex was then dialyzed, loaded onto a Q column and eluted with increasing NaCl. Lauryldimethylamine oxide (LDAO) was then added to the pooled fractions at a final concentration of 0.1% to release the HT-IpgC. The LDAO-treated IpaB/HT-IpgC or L-DBF/HT-IpgC complexes were then passed over a second IMAC column with the IpaB and L-DBF present in the flow-through. These were then dialyzed into PBS with 0.05% LDAO and stored at −80° C. LPS levels were determined using a NexGen PTS system with EndoSafe cartridges (Charles River Laboratories, Wilmington, MA). All proteins had LPS levels <5 EU/mg protein. Protein yields are typically 1 mg L-DBF per liter of culture, however, optimization of induction and production are still under way.

(3) Mouse S. flexneri 2a Pre-Exposure, L-DBF Vaccination, and Challenge.

S. flexneri 2a, S. flexneri 1b, or S. sonnei were cultivated on tryptic soy agar (TSA) plates containing 0.025% Congo red overnight. They were grown in tryptic soy broth (TSB) at 37° C., at 200 rpm until the absorbance (A600) reached 1. Bacteria were harvested by centrifugation and resuspended in PBS.

For the single pre-exposure study, female 6-8-week-old BALB/c mice (n=10/group) were exposed to PBS or 6×10⁴ CFU/30 μl S. flexneri 2a on day-60 (60 days prior to the first vaccination). For the two-dose pre-exposure study (Table 7), female 6-8-week-old BALB/c mice (n=14/group; 10 for challenge and 4 for pre-challenge immune response assessment) were used. The no treatment (NT) groups (sets A and B) were exposed to PBS on days −56 and −28. The treatment (T) groups (sets C and D) were exposed to 1×10⁵ CFU/30 μl of S. flexneri 2a on day-56 and 6×10⁵ CFU/30 μl of S. flexneri 2a on day-28. All mice were weighed once daily, and the health score monitored twice daily for 14 days after each pre-exposure. After recovering until day 0, three groups of the NT mice (set A) and three groups of the T mice (set C) were vaccinated (V) with 25 μg L-DBF in 30 μl per mouse on days 0, 14 and 28 while the other 6 groups were vaccinated with PBS (NV) (sets B and D). On day 56, one group (n=10) from each of the four sets were challenged with S. flexneri 2a, S. flexneri 1b, or S. sonnei (see Table 7). Mice were monitored twice daily for weight loss and health scores for two weeks. Mice were euthanized if their weight loss exceeded 25% of their original weight for more than 72 h or their blood glucose reached ≤100 mg/dL if they received a poor health score. The mouse animal protocols were reviewed and approved by the University of Kansas Institutional Animal Care and Use Committee Practices (protocol AUS 222-01).

### (4) IgG and IgA ELISAs

For the one dose pre-exposure mice, blood and fecal samples were collected on days 0, 13, 27, 41, 55 for determination of immunoglobulin titers. For the two dose pre-exposure mice, samples were collected at days −56, −28, −14, 0, 14, 28, 42 and 55. To determine anti-IpaD or -IpaB titers, wells were coated with 100 ng IpaB or IpaD in 100 μl PBS and incubated at 37° C. for 3 h. Wells were then blocked with 10% nonfat dried milk in PBS overnight. Sera were added to the wells in duplicates as the source of primary antibody and incubated for 2 h at 37° C. After washing with PBS containing 0.05% Tween 20, HRP-conjugated secondary antibodies (IgG(H+L), 1:1000; IgA, 1:500; IgM, 1:1000) were added and incubated for 1 h at 37° C. After an additional wash, OPD substrate (o-phenylenediamine dihydrochloride) was added and detected at 490 nm using an ELISA plate reader. Endpoint titers were determined by fitting antibody titrations to a five-parameter logistic model.

For determining the anti-LPS titers, wells were coated with 0.5 μg of S. flexneri 2a LPS in 100 μl 0.05 M carbonate buffer (pH 9.6) for 1 h at 37° C. After washing, wells were blocked with 10% nonfat dried milk in PBS for 1 hour at 37° C. After removing the milk and washing, sera were added to the wells in duplicates as primary antibody for overnight incubation at room temperature. The wells then were washed, the HRP-conjugated secondary antibody (IgG(H+L), 1:1000; IgA, 1:500; IgM, 1:1000) was added and the plate was incubated overnight at room temperature. After an additional wash, OPD substrate was added and the plates were read at 490 nm using an ELISA plate reader. Endpoint titers were determined by fitting antibody titrations to a five-parameter logistic model.

### (5) Serum Bactericidal Assay

The serum bactericidal assay (SBA) was modified by using high-throughput imaging of the bacteria on filter plates. Briefly, heat-inactivated serum, produced by pooling sera from each mouse of the group, was diluted two-fold with PBS in triplicate. A portion (90 μl) of the diluted serum and baby rabbit complement (Cedarlane, Burlington, NC) were added to each well of a 96-round well round bottom plate. A single colony of S. flexneri 2a grown on Congo Red TSA plate was sub-cultured in 10 ml of TSB at 37° C. with shaking at 200 rpm and grown until the A600 reached 0.2. S. flexneri (at 1×10⁴ CFU/10 μl) was added to each well and the plates placed at 37° C. with 200 rpm shaking for 1 h. A portion (20 μl) of each mix condition was transferred to ethanol-wetted wells of Millipore multiscreen HV filtration plates and the liquid removed by vacuum. Each plate was placed into a Ziploc bag and incubated at 37° C. and 5% CO₂ overnight. The next morning, Coomassie blue R-250 (100 μl of 0.01% solution) was added to each well and quickly removed by vacuum. A methanol-acetic acid destain solution (100 μl) was then added to each well with shaking at room temperature for 10 min. The destain solution was removed by vacuum and the plastic bottom of the filter plate was removed and allowed to air dry before counting. The CFUs were enumerated by a CTL (Cellular Technology Limited) immunospot reader.

For the competitive SBA, 4 μg of LPS of S. flexneri 2a 2457T, 2 μg of IpaB or IpaD in 45 μl PBS was added to the wells following two-fold dilutions done in triplicate. A portion (90 μl) of a pooled mixture of serum from pre-infected mice (1:512) or from L-DBF immunized mice (1:8 for the IpaB or IpaD; 1:64 for the LPS) was combined with baby rabbit complement in the appropriate wells. After gentle shaking at room temperature for 30 min, S. flexneri (1×10⁴ CFU/10 μl) was added to each well. The remaining steps were performed as above. The killing activity was measured by the following formula: Killing %=(Spots in NTNV well-spots in test well)/Spots in NTNV well. The number of spots in NTNV wells were statistically insignificantly different than the wells containing the complement and the bacteria. Thus, NTNV wells were considered as a baseline and a negative control group. SBA killing index (KI) was calculated by 10^{{log X1+[(Y50-Y1)×(logX2-logX1)]/(Y2-Y1)}}.

### (6) IFN-γ or IL-17A ELISpot Assays

Mouse cells were isolated from spleens and lungs. The cells were incubated for 24 h at 37° C. in the presence of 5 μg/ml IpaB or IpaD in plates coated with antibodies against IFN-γ or IL-17 using a FluoroSpot assay as per manufacturer's specifications (CTL). The cytokine secreting cells were quantified using a CTL Immunospot reader.

### (7) Cytokine Determinations

Splenocytes and lung cells were incubated with 10 μg/ml IpaB, IpaD or PBS for 48 h at 37° C. Supernatants were collected and analyzed with U-PLEX kits for cytokines according to manufacturer's specifications. Cytokine concentrations were determined using an MSD plate reader with associated analytical software (Meso Scale Discovery, Rockville, MD). While multiple cytokines were measured, the three that were focused on in this study are IL-17A, IFN-γ and IL-6 because the others that were tested did not show significant changes.

### (8) Statistics

GraphPad Prism 9.0.1 and Python were used for graphs and statistical analysis. ANOVA test was used for cytokine analysis. Log-rank (Mantel-Cox) tests were used for survival tests. Mann-Whitney tests were used for SBA analysis. *p<0.05; **p<0.01; ***p<0.001.

### Example 5: Impact of the TLR4 Agonist BECC438 on a Novel Vaccine Formulation Against Shigella Spp

Shigella causes a bloody diarrhea (dysentery) with 90 million cases globally each year resulting in approximately 164,000 deaths. Children are especially vulnerable with repeated episodes causing developmental and cognitive impairment. Shigella is also an important cause of diarrhea among travelers and military personnel who visit low- and middle-income countries. Shigella spp. are classified into S. dysenteriae (Group A), S. flexneri (Group B), S. boydii (Group C), and S. sonnei (Group D), which can be further divided into more than 50 serotypes based on the O-antigen component of lipopolysaccharide. S. flexneri, which includes 19 serotypes, is the primary species responsible for endemic shigellosis in developing countries. In contrast, S. sonnei, which comprises a single serotype, is predominant in more developed countries. Recent studies have shown that S. sonnei infections are increasing and are replacing endemic S. flexneri infections in some areas, which calls for strategic development of a serotype-independent vaccine to reduce the worldwide infection burden.

Antibiotics, such as fluoroquinolones, β-lactams and cephalosporins, have proven to be effective in reducing the risk of serious complications and death from shigellosis, however, increased resistance in developing countries has become a major concern in the treatment of shigellosis. Therefore, developing an effective and safe vaccine against shigellosis is an important strategy to reduce mortality as well as limiting antibiotic resistance. At present, there is no licensed vaccine against Shigella. There are some vaccine candidates, such as killed cell vaccines and live, attenuated vaccines are currently in clinical trials, however, low immunogenicity, lack of cross-protection, the strict storage conditions and the risks of contamination limit their use in developing countries.

To solve these issues, subunit vaccines, especially those comprised of the proteins from type three secretion system (T3SS), have been widely researched. T3SS is an important virulence factor used by Shigella to inject virulence effectors into host cells to facilitate cellular entry and to escape the host immune response. We have previously demonstrated that invasion plasmid antigen D (IpaD) resides at the tip of the Shigella T3SS injectisome needle and is required for control of type III secretion. We have also shown that the translocator protein IpaB associates with IpaD at the tip of needle and makes initial contact with host cells. IpaD and IpaB are highly conserved among the shigellae, which makes them targets for the development of a serotype-independent subunit vaccine against Shigella spp. Studies in our laboratory have established that these two proteins administered with the mucosal adjuvant dmLT (double mutant heat-labile enterotoxin from ETEC) can elicit cross protection against S. flexneri and S. sonnei when delivered intranasally (IN). To reduce production costs, we produced a genetic fusion of IpaB and IpaD, termed DBF. DBF administered with dmLT elicited a comparable immune response with protection similar to that stimulated by the mixture of IpaB and IpaD. Most importantly, DBF with dmLT administered IN protected mice from S. flexneri, S. sonnei and S. dysenteriae homologous and heterologous challenges.

Th17 responses, such as those elicited by dmLT, are believed to be important for protection against Shigella spp. Unfortunately, studies have shown that dmLT, when delivered IN, can cause Bell's palsy in humans. Since the LTA1 portion of the A subunit is responsible for generating the Th17 response, we genetically fused LTA1 to DBF to create a monomeric adjuvant-antigen conjugate L-DBF. The absence of the LT B subunit abrogates toxin binding to ganglioside GM1 on neuronal cells in the nasal passage and eliminates the risk of Bell's palsy. When delivered IN, L-DBF protects mice against homologous and heterologous Shigella spp. challenges. This protection is associated with significant Th1 and Th17 responses.

While L-DBF has been shown to successfully protect mice against lethal Shigella challenge, monomeric antigens often fail once they are introduced into human trials. Studies have shown a better response is elicited in humans when the antigen is presented as a multimer in the context of a nanoparticle. There are nanoparticle formulations currently being tested for use in intramuscular and intranasal vaccines. The most well-known multimerization method is the use of aluminum salts such as Alhydrogel, however, aluminum salts tends to skew the resulting immunity to a Th2 response, which is more aligned with the humoral response and not the balanced responses often required for clearing mucosal pathogens.

In this study, we examine the efficacy of DBF when formulated with the Bacterial Enzymatic Combinatorial Chemistry candidate 438 (hereafter referred to as BECC438), a novel TLR-4 agonist that is a biosimilar of monophosphoryl lipid A (MPL), which is approved for use in some human vaccines. BECC438 is a bis-phosphorylated hexa-acylated lipid A prepared from specifically engineered strains of Yersinia pestis. These studies were followed by an exploration of L-DBF formulated with BECC438 in an oil-in-water emulsion containing squalene, which has been shown to promote protection against influenza in an older population. In addition to fusion with LTA1, use of BECC438 further promotes a balanced Th1-Th2 immune response and increases protection elicited by low doses of L-DBF.

### a) Methods

### (1) Materials

pACYCDuet-1 plasmid, ligation mix and competent E. coli were from EMD Millipore (Billerica, MA). Restriction endonucleases were from New England Biolabs (Ipswich, MA). Chromatography columns were from GE Healthcare (Piscataway, NJ). All other reagents were from Sigma or Fisher Scientific and were chemical grade or higher. dmLT was a gift from J. Clements and E. Norton, Tulane School of Medicine, New Orleans, LA. S. flexneri 2a 2457T was a gift from A. T. Maurelli, University of Florida, Gainesville, Fl. Squalene was from Echelon Biosciences (Salt Lake City, UT).

### (2) Protein Production

IpaD, IpaB, DBF and L-DBF were made. Briefly, we utilized IpaD and IpaB proteins from S. flexneri 2a (strain 2457T). These proteins were chosen based on their high conservation across different serotypes of Shigella (IpaB: 98.9%; IpaD: 96%), as well as their known immunogenicity and involvement in the virulence of Shigella bacteria. The LTA (heat-labile toxin A1 subunit) used in our vaccine formulations was obtained from enterotoxigenic E. coli (ETEC). The final L-DBF preparation was dialyzed into PBS with 0.05% lauryl-dimethylamine oxide (LDAO) and stored at −80° C. LPS levels were determined using a NexGen PTS with EndoSafe cartridges (Charles River Laboratories, Wilmington, MA). All proteins had LPS levels <5 Endotoxin units/mg.

### (3) Preparation of Vaccine Formulations

Squalene (8% w/v) and polysorbate 80 (2% w/v weight) were mixed to achieve a homogenous oil phase. Polysorbate 80 was used as an emulsifying agent to stabilize the emulsion. Using a Silverson L5M-A standard high-speed mixer, 40 mM histidine (pH 6) and 20% sucrose were added to the oil phase and mixed at 7500 RPM followed by six passes in a Microfluidics 110P microfluidizer at 20,000 psi to generate a milky emulsion of 4×ME (MedImmune Emulsion). A similar method was used to make 4×NE (a new squalene-based emulsion formulated in our laboratory using a different aqueous phase) using 40 mM MOPS/20 mM Na₂HPO₄ (pH 7.6). To make the DBF with ME or NE, the protein was added to the emulsion to a final concentration of 0.67 mg/ml, vortexed and allowed to incubate overnight at 4° C. To the emulsions containing BECC438, BECC438 (2 mg/ml) was prepared in 0.5% triethylamine by vortexing followed by sonicating for 30 min in a 60° C. water bath sonicator until the BECC438 was completely dissolved. The pH of BECC438 solution was adjusted to 7.2 with 1 M HCl. To make the BECC438 with ME or NE formulation, the BECC438 was mixed with ME or NE and the sample vortexed for 2 min followed by an overnight incubation at 4° C. The next day, DBF or L-BDF was mixed with the appropriate base formulation at a volumetric ratio of 1:1 to achieve desired final antigen concentration.

### (4) Preparation of DBF BECC438/Chi-C48/80 Formulation

To make chitosan nanoparticles, 1 gm of chitosan was added to 10 mL of a 1 M NaOH solution and stirred for 3 h at 50° C. The chitosan solution was then filtered through 0.45 μm membrane and the solid fraction was washed with 20 mL of MilliQ water. The recovered chitosan was resuspended in 200 mL of 1% (v/v) acetic acid solution and stirred for 1 h. The solution was filtered through 0.45 μm membrane and 1 M NaOH was added to adjust the pH to 8.0, resulting in purified chitosan. Purified chitosan was vacuum dried for 24 hours at 40° C. The mast cell activating agent compound 48/80 (C48/80) was then loaded on the chitosan nanoparticles (Chi) adding dropwise 3 ml of an alkaline solution (5 mM NaOH) containing C48/80 and Na₂SO₄ (0.3 mg/mL and 2.03 mg/mL, respectively) to 3 mL of a chitosan solution (1 mg/mL in acetic acid 0.1%) with high-speed vortexing. The Chi was formed using magnetic stirring for an additional 1 h. Chi was then collected by centrifugation at 4500×g for 30 min and the pellet resuspended in MOPS buffer (20 mM, pH 7). The DBF in PBS was exchanged into MOPS buffer (20 mM, pH 7) using an Amicon Ultra-4 centrifugal filter. To make DBF BECC438/Chi-C48/80, the nanoparticles were mixed with BECC438 by vortexing and incubating for 10 min. DBF was then added, mixed by vortexing and incubated for 2 h at 4° C.

### (5) Mouse Immunization and Sample Collection

The mouse animal protocols were reviewed and approved by the University of Kansas Institutional Animal Care and Use Committee Practices (protocol AUS 222-01). Female 6-8 weeks BALB/c mice were used in this study (n=10 or 14/group). The negative control group was vaccinated with PBS (30 μl) either intranasally (IN), intramuscularly (IM) or intradermally (ID) depending upon the route of the experimental groups The positive control group was vaccinated IN with 20 μg DBF+2.5 μg dmLT or 25 μg L-DBF. For the IM trials, the indicated formulations were prepared in 30 μl volume and delivered to the inner thigh with ½ cc LO-DOSE U-100 Insulin Syringe with 28G ½″ needle. For the IN trials, the indicated formulations were prepared in 30 μl volumes and delivered with a pipette tip to the nares. Mice were immunized on Days 0, 14 and 28. For the ID trial, 100, 250 and 500 ng DBF with 5 μg BECC438 were diluted to 50 μl per mouse as per.

(6) Shigella flexneri Challenge Studies

S. flexneri 2a 2457T was streaked onto tryptic soy agar containing 0.025% Congo Red, incubated at 37° C. overnight, and then subcultured into tryptic soy broth (TSB) for growth at 37° C. until the absorbance at 600 nm (A600) reached 1.0. Bacteria were harvested by centrifugation, resuspended in PBS, and diluted to the desired concentration in a 30 μl volume for IN challenge. Mice were challenged with 1-10×10⁶ CFU per mouse of S. flexneri 2457T on Day 56 (four weeks after the final immunization). Mice were monitored twice a day for weight loss and health score for two weeks. Mice were euthanized when they lost more than 25% of their original weight for more than 72 h or their health score was considered poor and accompanied by a blood glucose level ≤100 mg/dL. All remaining mice were euthanized on Day 14 post-challenge.

### (7) Antigen Specific IgG and IgA ELISAs

Fecal pellets and 100 μl blood obtained by the orbital sinus route were collected on Days 27, 41 and 55. Anti-IpaD and -IpaB IgG and IgA titers were determined. Briefly, microtiter plate wells were coated with 100 ng IpaB or IpaD in 100 μl PBS and incubated at 37° C. for 3 h. Wells were the blocked with 10% nonfat dry milk in PBS overnight. Sera were added to the wells in duplicates as the primary antibody for 2 h incubation at 37° C. After washing with PBS-0.05% Tween an HRP-secondary antibody (IgG(H+L), 1:1000; IgA, 1:500) was added and incubated for 1 h at 37° C. After an addition wash, OPD substrate (o-phenylenediamine dihydrochloride) was added and detected at 490 nm by ELISA plate reader. Endpoint titers were determined by fitting antibody titrations to a five-parameter logistic model.

### (8) Enumeration of IFN-γ or IL-17 Secreting Cells

Mouse necropsies in the IM studies were performed on Day 3 after challenge with four mice from each group sacrificed to collect, individually, the lung, spleen, for immunology tests. Samples from mice in DBF with BECC438 alone via IN route study were collected on Day 14 for the mice that survived. Samples from mice in IN studies of BECC438 optimal formulations were collected at Day 52 from the pre-challenge vaccinated group. Mouse cells isolated from spleens and lungs were incubated for 24 h at 37° C. in the presence of 5 μg/ml IpaB or IpaD in plates coated with antibodies against IFN-γ or IL-17 using a FluoroSpot assay as per manufacture's specifications (Cellular Technology Limited). The cytokine secreting cells were quantified using a CTL immunospot reader.

(9) Quantification of Secreted Cytokines after Stimulation

Splenocytes and lung cells were incubated with 10 μg/ml IpaB, IpaD or PBS for 48 h at 37° C. Supernatants were collected and analyzed with U-PLEX kits for cytokines: IFN-γ, IL-17A, IL-6 and TNF-α. Cytokine concentrations were determined using an MSD plate reader with associated analytical software (Meso Scale Discovery, Rockville, MD). While multiple cytokines were measured, the two that are focused on in this report are IL-17A and IFN-γ as others did not show significant changes.

### (10) Statistical Analysis

Graphs were created using GraphPad Prism 9.0.1. Lung cytokine secretion were rescaled to the range between zero and one using min-max normalization [Yₙₒᵣₘₐₗ=(Y_{origin}−Yₘᵢₙ)/(Yₘₐₓ−Yₘᵢₙ)]. Differences among unvaccinated (PBS) mice and antigen vaccinated mice were analyzed using ANOVA. A p value of less than 0.05 was considered significant for all comparisons. *p<0.05; **p<0.01; ***p<0.001. For bacterial challenges, vaccinated groups were compared to PBS with Log-rank (Mantel-Cox) tests in GraphPad Prism.

### b) Results

(1) Intramuscular or Intradermal Immunization with DBF+BECC438 Formulations does not Protect Mice Against Lethal S. flexneri Challenge.

IN delivery of DBF+dmLT protects against an otherwise lethal S. flexneri challenge. However, this formulation elicited poor protection when delivered IM. In this study, we wanted to determine whether bis-phosphorylated BECC438 could improve the protective efficacy of this formulation when delivered IM. BECC438 has been shown to be protective against Yersinia pestis and Influenza A when delivered IM with the appropriate antigen. In our first experiment, mice were vaccinated IM with 0.1 to 40 μg DBF+5 μg or 50 μg BECC438 (Table 9), or ID with 100, 250 or 500 ng DBF+5 μg BECC438. As a positive control group, mice were vaccinated IN with 20 μg DBF+2.5 μg dmLT. All the mice vaccinated IN with DBF+dmLT survived the otherwise lethal challenge while all the mice vaccinated with PBS succumbed to the infection. Likewise, none of the groups vaccinated IM using BECC438 as the adjuvant demonstrated greater than 50% survival with no significance detected between the groups (study of 5 μg BECC438: p>0.066; study of 50 μg BECC438: p>0.35). Similar results were found when the formulations with BECC438 were delivered ID with each of the formulations providing <30% protection by this route.

TABLE 9
Vaccine efficacy in response to a DBF dose escalation
with formulations containing either 5 or 50 μg BECC438

| DBF | VE % of 5 μg | VE % of 50 μg |
|---|---|---|
| concentration | BECC438 | BECC438 |
| 40 μg DBF | 17 | 33 |
| 15 μg DBF | 0 | 17 |
| 5 μg DBF | 17 | 0 |
| 1.5 μg DBF | 0 | 0 |
| 0.5 μg DBF | 33 | 17 |
| 0.1 μg DBF | 0 | ND |

Mice (n = 6) were vaccinated IM with the indicated formulations. They were then challenged with 6 × 10⁶ CFU (in 30 μl) S. flexneri 2a 2457T. Vaccine Efficacy (VE) is shown where VE (%) = 1 − Attack Rate Vaccinated/Attack Rate Unvaccinated (PBS control) where all PBS vaccinated mice died. All mice vaccinated IN with 20 μg DBF + 2.5 μg dmLT survived and this was used as the positive control in this experiment.

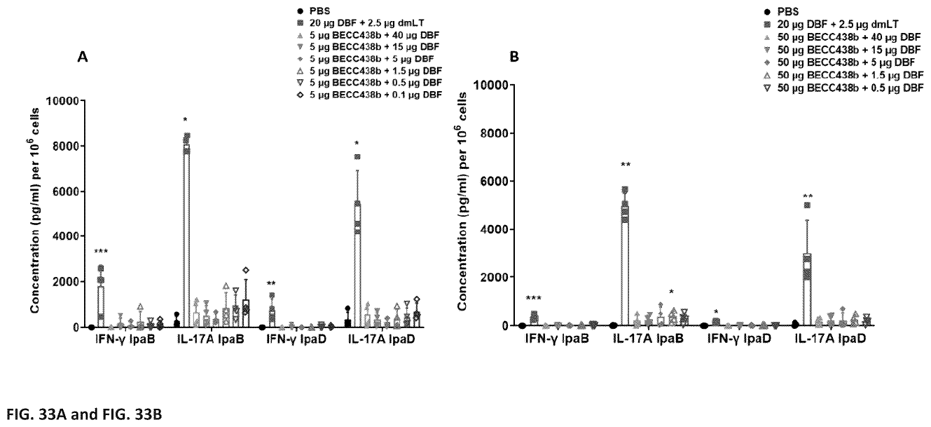

To determine whether BECC438 admixed with DBF stimulated the T cell-related cytokine responses when administered via the IM route, the lungs from four of the surviving mice vaccinated with the DBF formulation were sampled early on Day 3 post-challenge (FIG. 33). It should be noted that by late afternoon of Day 3, all the remaining mice vaccinated with PBS had died. Secretion of IFN-γ and IL-17A from the harvested lung cells was then assessed following stimulation with IpaB and IpaD. In contrast to the prominent levels of IFN-γ and IL-17A secreted by lung cells from the DBF+dmLT IN group, the secretion of these cytokines was not observed for the lung cells from the mice vaccinated IM with DBF+BECC438 (FIG. 33). Thus, it appears that when delivered IM, BECC438 lacks the functional adjuvanticity needed to induce protective T cell immunity within the mouse lung against S. flexneri infection.

(2) IN Immunization with BECC438 Admixed with DBF Induces Only Partial Protection and Elicits Low Levels of Cytokines in Splenocytes

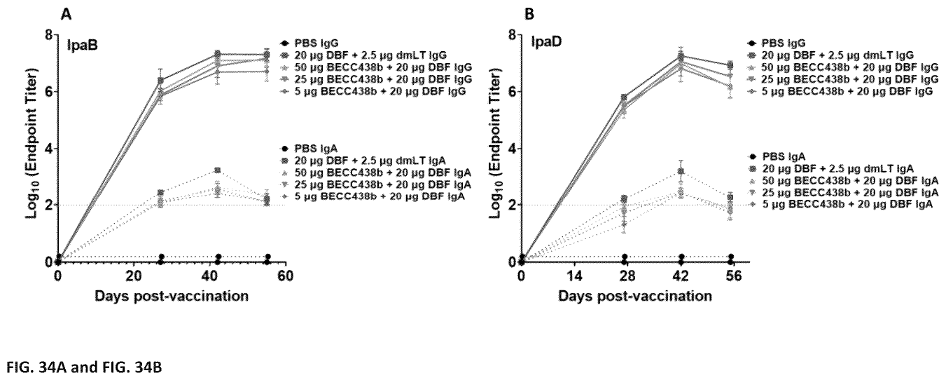

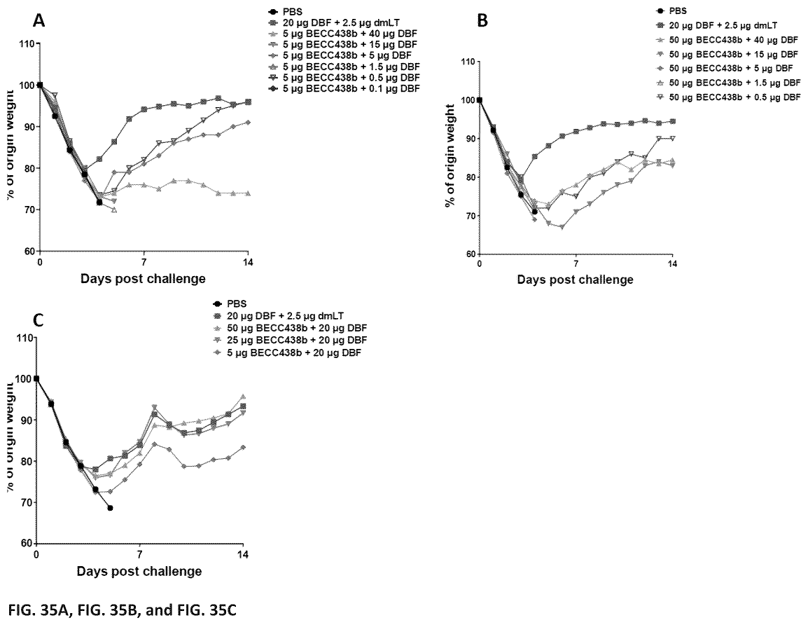

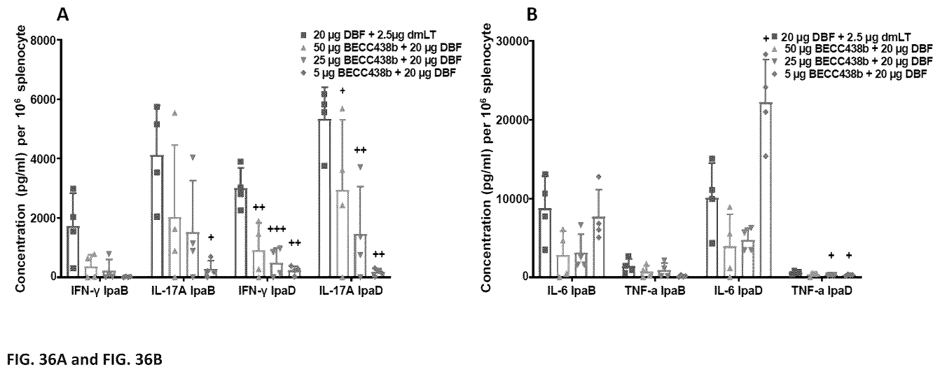

To determine whether BECC438 could induce T cell responses via mucosal immunity, mice (n=10) were vaccinated IN with 20 μg DBF+2.5 μg dmLT (positive vaccine control) or with 20 μg DBF+5, 25 or 50 μg BECC438. All mice vaccinated with DBF formulations had similar levels of anti-IpaB or -IpaD IgG or IgA (FIG. 34). While 90% of mice vaccinated with the DBF+dmLT survived this challenge, the groups vaccinated with BECC438 had <40% survival (Table 10). It should be noted, however, that the surviving mice vaccinated with DBF+25 or 50 μg BECC438 showed weight recovery trends similar to of those mice in the positive control group. This was not observed for the IM or ID administration groups (FIG. 35). Notably, the surviving mice in the IM vaccinated groups experienced delayed weight gain by at least 24 hours compared to the positive control groups. The weight recovery trends observed in the surviving mice vaccinated with DBF+25 or 50 μg BECC438 indicated a positive outcome, indicating that these vaccination regimens were effective in promoting recovery and restoring normal health parameters in the mice. To identify whether the improved weight recovery was from the adjuvanticity of BECC438 via the IN route and to investigate the BECC438 effects on cytokine levels, we assessed IL-17A, IFN-γ, IL-6 and TNF-α levels secreted upon stimulation of isolated lung cells and splenocytes from mice remaining on Day 14 post-challenge (FIG. 36). In this case, the lung cells did not provide a clear picture of what was occurring, so we're reporting here on the data obtained using the splenocytes. Furthermore, because all the mice vaccinated with PBS had died, we did not have a proper negative control to provide the usual statistical comparisons. Therefore, we compared the mice vaccinated with DBF+BECC438 with the positive control mice vaccinated with DBF+dmLT. After stimulation with IpaB or IpaD, high IL-17A and IFN-γ levels were detected in splenocyte supernatants from the mice vaccinated IN with dmLT as the adjuvant (FIG. 36A). When compared to these positive control mice, only the splenocytes from mice vaccinated with DBF+25 μg or 50 μg BECC438 secreted similar IL-17A levels after stimulation with IpaB (p>0.05), however, all three of the BECC438 groups secreted IFN-γ at levels similar to that of the positive control (p>0.05). Conversely, when stimulated with IpaD, all BECC438 groups had statistically lower levels of IFN-γ and IL-17A than the positive control. Consistent with the IL-17A and IFN-γ levels, the levels of IL-6 and TNF-α secreted were similar to the positive control in all cases after stimulation with IpaB (FIG. 36B). When stimulating with IpaD, the 5 μg BECC438 group showed high levels of IL-6, which is consistent with the inability to regain weight at the rates seen for the other groups (FIG. 36B). Nevertheless, the protective efficacy of all the BECC438-treated groups was lower than for the DBF+dmLT vaccinated mice.

TABLE 10
Vaccine efficacy of 20 μg DBF with a BECC438
dose escalation administered by the IN route

| BECC438 | |
|---|---|
| concentration | VE % |
| 50 μg BECC438 | 40 |
| 25 μg BECC438 | 30 |
| 5 μg BECC438 | 30 |

Mice (n = 10) received IN vaccination using the specified formulations. Subsequently, they were exposed to a challenge of 1 × 10⁷ CFU of S. flexneri 2a 2457T, administered in 30 μl. Vaccine Efficacy (VE) is represented as a percentage, calculated using the formula VE (%) = 1 − (Attack Rate Vaccinated/Attack Rate Unvaccinated) where the control group receiving PBS did not survive. As a positive control, mice were IN vaccinated with 20 μg DBF + 2.5 μg dmLT, resulting in 90% survival in this specific challenge experiment.

(3) IN Immunization with Multimeric DBF+BECC438 Formulated in an Oil-In-Water Emulsion Induces Partial Protection Against Shigella Infection.

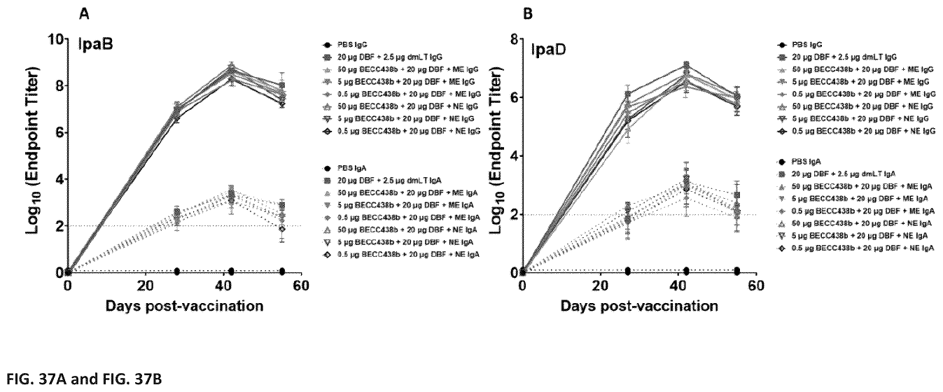

As mentioned above, subunit vaccines for use in humans are typically best presented using a multimeric antigen formulation. Therefore, because there were indications that DBF+BECC438 can elicit some level of protection relative to the negative control with cytokine responses that were on par in some cases with those seen for the positive control, we developed two oil-in-water formulations to boost the T cell responses to this antigen-adjuvant combination. Toward this end, squalene-based ME or NE was included as part of the IN formulations with 20 μg DBF and three distinct doses of BECC438 (Table 11). Regardless of the oil-in-water formulation used, the resulting anti-IpaB and anti-IpaD IgG and IgA titers were essentially equivalent (FIG. 37). All the mice vaccinated in the DBF+dmLT positive control survived the challenge while all mice in the PBS vaccinated group succumbed to the challenge. The protective efficacies of mice vaccinated with DBF+0.5 μg, 5 μg, 50 μg BECC438 in the ME formulations were 60, 50 or 70%, respectively, with the DBF+5 μg BECC438 in ME group having somewhat slower weight recovery compared to other groups. In contrast, all the groups vaccinated with DBF+BECC438 formulated with NE had 40% or less survival (Table 11), indicating that the aqueous phase of the oil-in-water emulsions can have an effect on formulation efficacy.

TABLE 11
Vaccine efficacy of 20 μg DBF with a BECC438
dose escalation in formulations containing ME
or NE nanoemulsions after delivery via the IN route.

| | |
|---|---|
| BECC438 | VE % |

| concentration | ME | NE |
|---|---|---|
| 50 μg BECC438 | 70 | 40 |
| 5 μg BECC438 | 50 | 30 |
| 0.5 μg BECC438 | 60 | 40 |

Mice (n = 10) received IN vaccination using the specified formulations. Following vaccinations, they were challenged with 1 × 10⁷ CFU of S. flexneri 2a 2457T (in 30 μl). The Vaccine Efficacy (VE) is calculated as VE (%) = 1 − (Attack Rate Vaccinated/Attack Rate Unvaccinated), with the unvaccinated group receiving PBS serving as the control. All the mice in the PBS group succumbed to the challenge. In this experiment, the positive control consisted of IN vaccination with 20 μg DBF + 2.5 μg dmLT, resulting in a remarkable 100% survival rate.

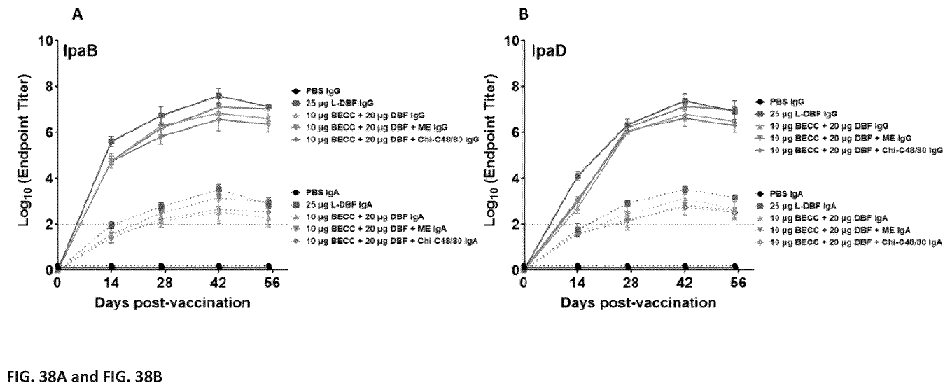

Because some of the mice did not tolerate DBF+50 μg BECC438 ME nanoemulsion formulation particularly well (30% of mice suffered from illness during these vaccinations), we reduced the BECC438 to 10 μg to better control the overall immunostimulatory potential. At the same time, other ongoing studies within our laboratory had shown protective efficacy with the use of chitosan prepared with the mast cell-activating adjuvant C48/80, which we call Chi-C48/80. This led us to consider this as a nanoparticle formulation alongside the ME nanoemulsion. Furthermore, prior to the completion of these studies, we developed a self-adjuvanting form of DBF called L-DBF, which is a genetic fusion of LTA1 and DBF that protects mice as well as admixed DBF+dmLT. We therefore used L-DBF as the positive control for these studies moving forward. When we vaccinated mice with L-DBF or 20 μg DBF+10 μg BECC438 admixed with either ME or with Chi-C48/80, we found that the resulting anti-IpaB and anti-IpaD IgG and IgA titers were essentially equivalent (FIG. 38). In this experiment, the mice vaccinated with L-DBF had 80% survival following a high dose challenge, while none of the mice vaccinated with PBS survived. Unfortunately, the reduction of BECC438 to 10 μg lowered the protective efficacy to 30% and none of the mice from the Chi-C48/80 group survived the challenge.

(4) Optimized BECC438 Formulations can Elicit IL-17 and IFN-γ Secretion in Lung Cells when Administered IN

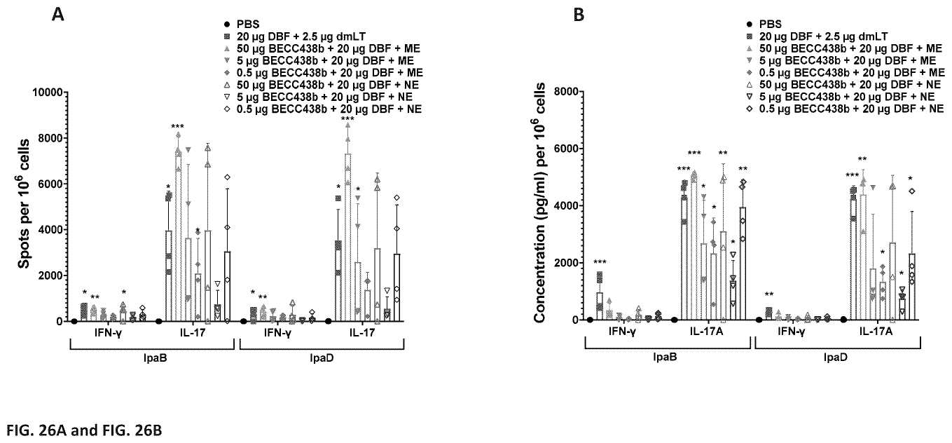

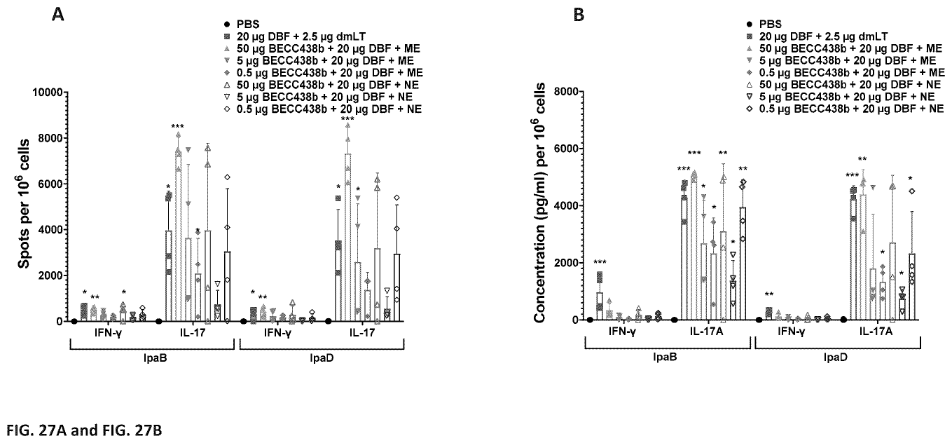

To understand the reasons for the low efficacy of these BECC438-containing formulations relative to DBF+dmLT or L-DBF positive controls, we assessed the T cell related cytokines elicited by the ME, NE and Chi-C48/80 formulations. In the first trial, the frequency of IFN-γ and IL17 secreting cells were enumerated from mice vaccinated with either the ME or NE formulations (FIG. 26A). The frequencies of the IFN-γ- and IL-17-secreting T cells from the lungs of mice vaccinated with DBF+dmLT and the 20 μg DBF+50 μg BECC438 with ME groups were significantly increased following stimulation with IpaB and IpaD. When the levels of secreted cytokines after stimulation were assessed, high levels of IL-17A were secreted from lung cells from most of the groups (FIG. 26B), but only the DBF+dmLT group showed significant IFN-γ secretion. In the second trial, 25 μg L-DBF was used as the positive control for comparison with 20 μg DBF+10 μg BECC438 alone, with ME or with Chi-C48/80 and the frequency of lung IFN-γ- and IL-17-secreting cells was quantified after stimulation with IpaB and IpaD (FIG. 27A). The frequency of IL-17 secreting cells from the L-DBF group and the 20 μg DBF+10 μg BECC438 with ME were significantly higher compared to PBS group after stimulation by IpaB and IpaD. In contrast, none of the groups had a higher frequency of IFN-γ-secreting cells compared to PBS group (25 μg L-DBF vs. PBS: IpaB IFN-γ p=0.63). In contrast, when the levels of cytokines secreted after stimulation of the lung cells with IpaB or IpaD were assessed, only the Chi-C48/80 failed to show a significantly higher level of IL-17A secretion (FIG. 27B) and only the L-DBF group showed significantly greater IFN-γ secretion compared to PBS group. Thus, the only groups within these two sets of experiments that contained the LTA1, either in the form of dmLT or as L-DBF, showed significant levels of secreted IL-17A and IFN-γ upon stimulation with both IpaB and IpaD. On the other hand, BECC438+DBF in the context of the ME nanoemulsion, did demonstrate strong IL-17 responses after stimulation with IpaB and IpaD. Meanwhile, the Chi-48/80 group failed to show elevated IL-17 and IFN-γ in any of the cases, indicating that BECC438+DBF is best presented as part of an oil-in-water nanoemulsion.

(5) The Presence of LTA1 Fused with DBF Enhances the Protection Seen for the Optimized BECC438 Formulations by Eliciting Greater Cytokine Responses.

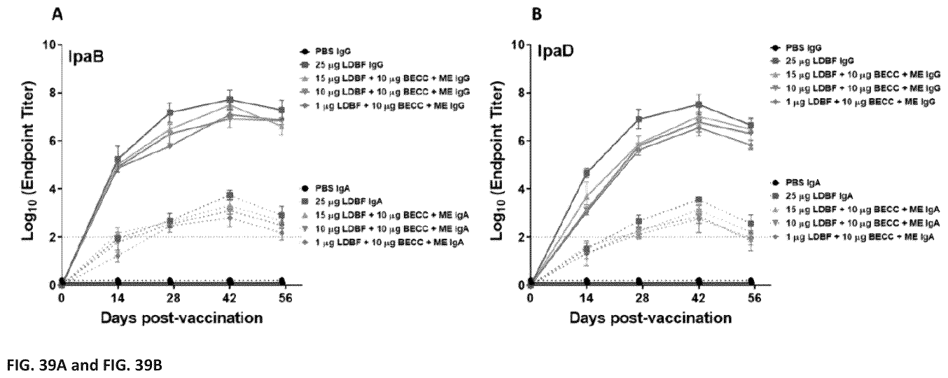

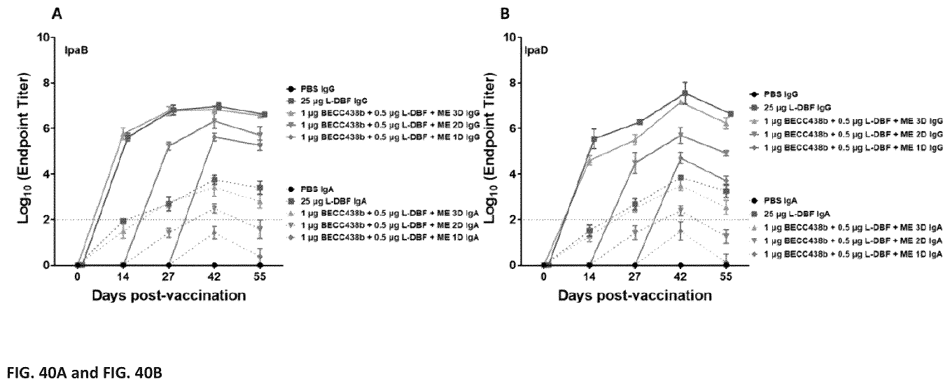

To increase the protective efficacy against the intracellular pathogen Shigella, L-DBF (instead of DBF) was used as a component of the BECC438 with ME formulation. Because of the high efficacy of 25 μg L-DBF alone, we believed that BECC438 might allow for it to be used in dose- and antigen-sparing amounts or with fewer immunizations required. Two separate experiments were performed here with one using 1, 10 or 15 μg L-DBF+10 μg BECC438 with ME in a prime-boost-boost regimen and another using 0.5 μg L-DBF+1 μg BECC438 with ME in prime only, prime-boost or prime-boost-boost regimens. In the first L-DBF dose escalation study, >90% of mice vaccinated with any of the L-DBF doses survived (Table 12) with similar IgG and IgA levels (FIG. 39). However, we have observed that the administration of 10 μg of BECC in combination with either 15 μg or 10 μg of L-DBF in ME induced certain side effects, which indicated that 1 μg L-DBF+10 μg BECC438 with ME is a considerable formulation. The second study used a lower BECC438 and L-DBF dose that can still afford protection to assess the number of boosts required for protection. The group given two boosters demonstrated 60% protection, while the single boost group was afforded 50% protection. The anti-IpaB IgG levels were similar for the one and two boost groups while the anti-IpaB IgA for both were either below or just above (at the second boost) baseline (FIG. 40). In contrast, the anti-IpaD IgG levels of the single boost group were much lower than the group with two boosts with the anti-IpaD IgA being very low as was seen with the anti-IpaB IgA. Unfortunately, the prime administration alone did not induce an immune response sufficient to protect against a subsequent challenge (Table 13). For this group, IgA was not above the baseline level and the IgG levels were much lower than the prime-boost-boost group. Only the anti-IpaB IgG levels approached that of the groups with boosters. Ultimately, it was found that BECC438 with ME allowed for the use of lower doses of L-DBF to provide protection (down to 1 μg), but that optimal protection still required a prime-boost-boost immunization schedule.

TABLE 12
Vaccine efficacy for an L-DBF dose escalation formulated
with BECC438 and ME after IN administration.

| Formulation | VE % |
|---|---|
| 10 μg BECC438 + 15 μg L-DBF + ME | 90 |
| 10 μg BECC438 + 10 μg L-DBF + ME | 100 |
| 10 μg BECC438 + 1 μg L-DBF + ME | 90 |

Mice (n = 10) were vaccinated IN with the indicated formulations. They were then subjected with 1 × 10⁶ CFU (in 30 μl) S. flexneri 2a 2457T. Vaccine Efficacy (VE) is shown where VE = 1 − Attack Rate Vaccinated/Attack Rate Unvaccinated (PBS control) where the control group receiving PBS vaccination witnessed mortality in all mice. Positive control mice vaccinated IN with 25 μg L-DBF where all the mice survived.

TABLE 13
Vaccine efficacy of L-DBF formulated with BECC438 and ME
delivered via IN route using different booster regimens.

| Formulation | Booster | VE % |
|---|---|---|
| 1 μg BECC438 + 0.5 μg L-DBF + ME | 2 | 60 |
| 1 μg BECC438 + 0.5 μg L-DBF + ME | 1 | 50 |
| 1 μg BECC438 + 0.5 μg L-DBF + ME | 0 | 30 |

A total of 10 mice in each group were intranasally (IN) vaccinated using the specified formulations. Subsequently, they were exposed to a challenge of 1 × 10⁶ CFU (in 30 μl) of S. flexneri 2a 2457T. The Vaccine Efficacy (VE) is depicted as VE (%) = 1 − (Attack Rate Vaccinated/Attack Rate Unvaccinated), with the control group receiving PBS experiencing mortality among all mice. Notably, the positive control mice were IN vaccinated with 25 μg L-DBF, resulting in the survival of all mice following the challenge.

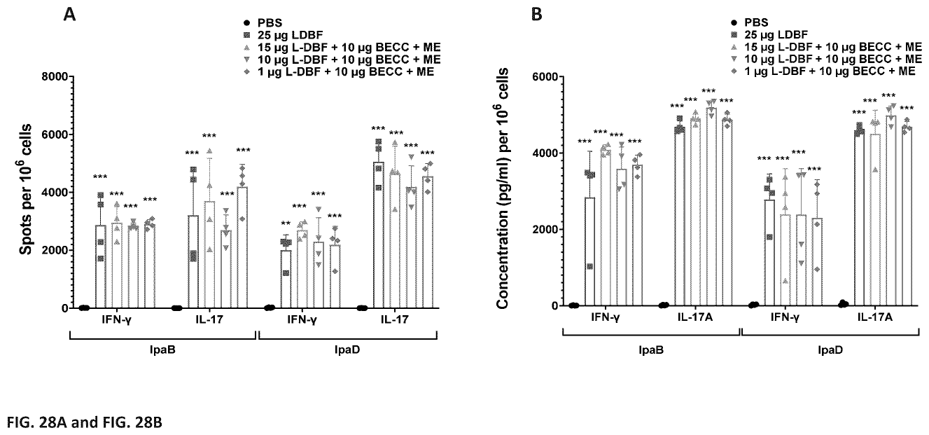

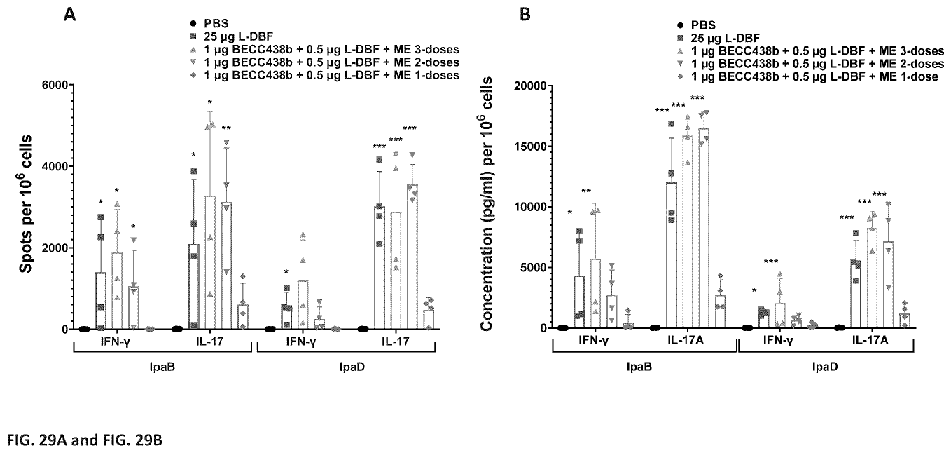

To understand the effects of these modified formulations on the cellular immune response, we assessed their effect on the IFN-γ and IL17 cytokine responses of isolated lung cells. In prior work, we found that 10 μg L-DBF or 1 μg L-DBF alone does not protect mice against Shigella infection and elicits lower IFN-γ and IL-17 responses when compared to 25 μg L-DBF. This lack of protection contrasts with what is seen when these doses of L-DBF are formulated with BECC438 and ME (Table 12). We therefore chose to assess the frequency of IFN-γ- and IL17-secreting cells from the lung upon antigen stimulation following vaccination using the dose escalation and the booster regimens described above (FIGS. 28A and 29A). After stimulating with IpaB and IpaD, all L-DBF+BECC438 with ME vaccinated groups from the dose escalation study groups, regardless of concentration, induced significantly higher frequencies of IFN-γ or IL17 secreting cells compared to those from the PBS group (FIG. 28A). In contrast, in the prime-boost regimen study, only the positive control and prime-boost-boost groups had significantly higher frequencies of IFN-γ and IL17 secreting cells after stimulation than the PBS control, though the prime-boost regimen did give rise to an elevated frequency of IL17 secreting cells after stimulation with either IpaB or IpaD (FIG. 29). Interestingly, while there was an increase in the frequency of IFN-γ secreting cells from the prime-boost lung cells when they were stimulated with IpaB, no significance was seen in the frequency of IFN-γ cells after stimulation with IpaD (FIG. 29A). When the levels of secreted IFN-γ and IL-17A were quantified for the lung cells after stimulation, all groups vaccinated with L-DBF, regardless of concentration, had significantly higher levels of both IFN-γ and IL-17A than the lung cells from the PBS group (FIG. 28B). In contrast, only the positive control and prime-boost-boost groups had significantly higher levels of both cytokines. The results indicate that even low levels of LTA1 on L-DBF can enhance the stimulation of Th1 and Th17 cytokines in the presence of BECC438. This is especially true for the increased secretion of IFN-γ which is necessary for a protective response against Shigella spp.

(6) Correlation of Protection with the Secretion of IFN-γ and IL-17 after Stimulating with IpaB or IpaD.

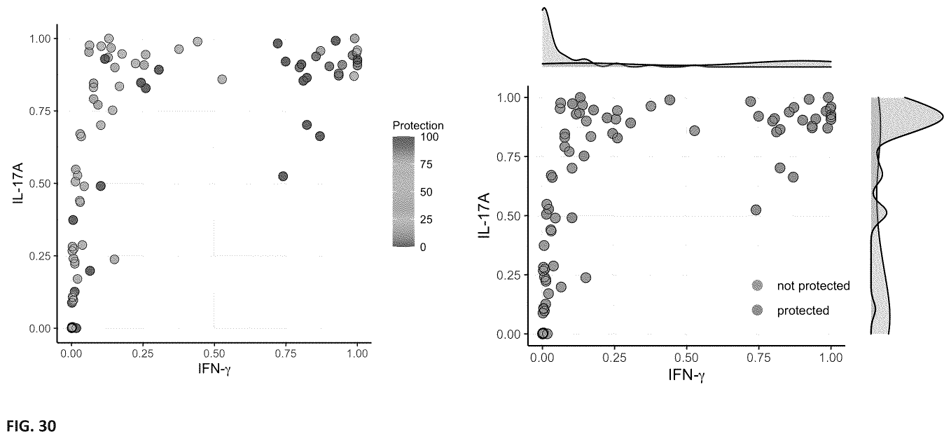

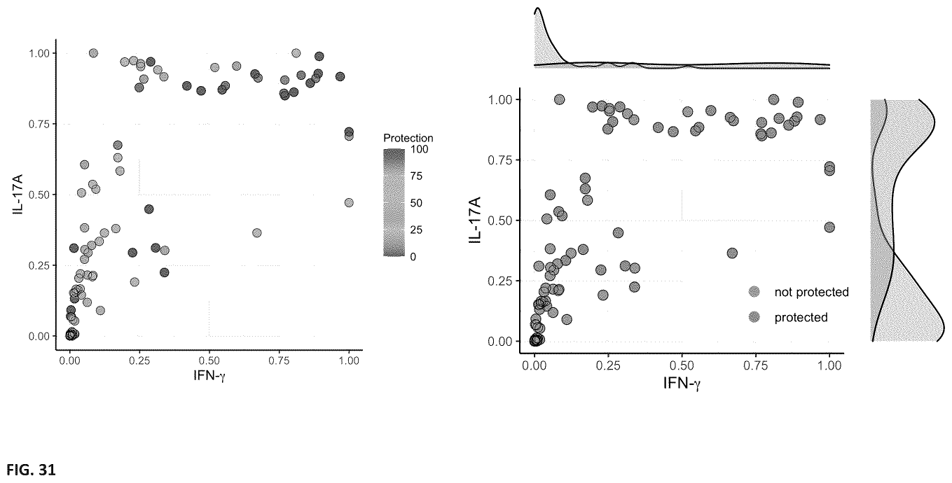

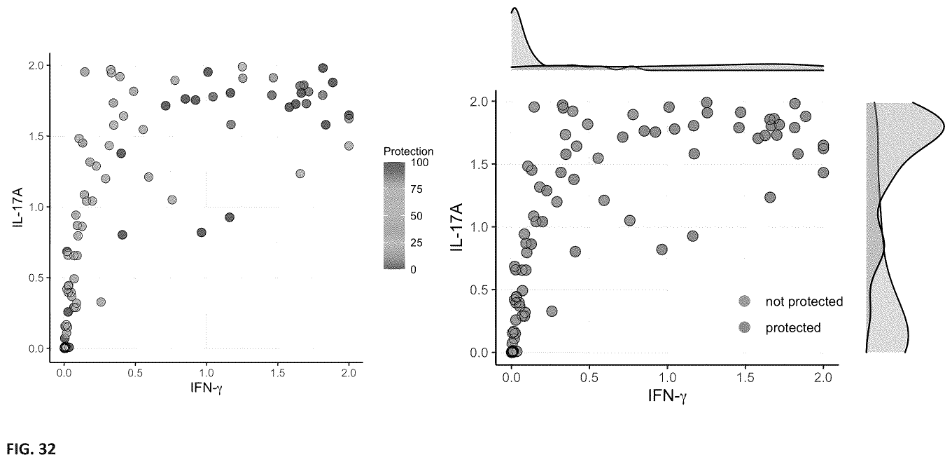

Based on the findings presented here, we evaluated the correlation between vaccine-mediated protection with the secretion of IFN-γ and IL-17A by lung cells following stimulation with IpaB or IpaD. We first normalized the data into a 0 to 1 scale and established a model involving IFN-γ (X) and IL-17A (Y) versus protection (FIGS. 30 to 32). IL-17 stimulated by IpaB was a strong predictor that mice would survive an otherwise lethal challenge with Shigella (FIG. 30), however, IpaB induced IL-17 secretion alone did not appear to predict the highest level of survival in the challenge. In general, IpaB induction of IFN-γ secretion by lung cells (>0.5) appeared to be required to reach >60% protection. IpaD induction of IL-17 and IFN-γ secretion also showed a significant positive correlation with protection, albeit this correlation was lower than for IpaB, and both cytokines were required to provide a successful immunization (FIG. 31). We also added the normalized outcomes seen for IpaB and IpaD together and established a correlation curve between these cytokines and the vaccine-mediated protection (FIG. 32). In general, the cut-off point for the cytokine level to predict a protective outcome was ˜1.0, which indicates that a successful vaccine candidate is able to induce both IL-17 and IFN-γ and that both IpaB and IpaD contribute to this outcome.

### c) Discussion

Shigellosis can be fatal for infants and children under five years of age, especially for those living in low-income countries where it is endemic and there is limited access to basic life-saving treatments and sanitation infrastructure. Existing and emerging antibiotic resistance among the shigellae calls for an effective vaccine against shigellosis, but none have been licensed to date. A broadly protective vaccine as part of an optimized formulation tailored for use in humans is urgently needed. We tested subunit vaccine candidates in which IpaD and IpaB of the Shigella T3SS were genetically fused (DBF) using the mouse lethal pulmonary model and found that when administered IN with dmLT or as a fusion that contains LTA1 moiety of dmLT (L-DBF) and found they have a high protective efficacy. Additionally, due to the conservation of IpaD and IpaB across different Shigella species, our vaccine candidates can also induce cross-protective immune response against multiple Shigella serotypes. Unfortunately, monomeric antigens tend to be poor at inducing strong protective responses in humans and a multimeric presentation is often required. We therefore chose to explore the use of different formulations (ME, NE and Chi-C48/80) with our DBF and L-DBF vaccine candidates to identify presentation and adjuvant combinations that can be of use in developing a human vaccine against Shigella. ME is emulsion-based systems with small droplet sizes (˜ 140 nm) which improves antigen stability, uptake, and immunogenicity, while Chi-C48/80 is a chitosan derivative that acts as an adjuvant that enhances immune responses through its interactions with immune cells. The new squalene-based emulsion NE had been indicated a smaller average size (˜ 65 nm) compared to ME.

We have investigated the adjuvant effects and physical properties of ME and Chi-C48/80 in the context of enhancing the immune response against Salmonella and Pseudomonas infections. In this study, ME proved to be a more effective adjuvant for enhancing the immune response against Shigella infection. We found NE and Chi-C48/80 did not demonstrate the same level of adjuvant activity as ME.

We tested multiple delivery routes and found that IN administration best induced a mucosal immune response that protected mice from lethal challenge, though a certain degree of protection can be elicited by IM and ID administration. Here we tested the IM, ID and IN routes by formulating DBF with BECC438, a novel bisphosphorylated lipid A adjuvant that is a TLR4 agonist and a biosimilar of MPLA, which is approved for use in some human vaccines. When DBF at different concentrations was administered IM and ID with BECC438, little protection was seen except for with IM at the highest DBF concentrations (Table 9). In contrast, DBF with BECC438 delivered IN did provide some protection though not as much as DBF with dmLT. In this case, however, IN administration did elicit IL-17 and IFN-γ responses, indicating that it did promote responses deemed necessary for protection against Shigella (Table 10 and FIG. 33). We thus chose to reformulate the DBF with BECC438 to present the antigen in a polymeric form by including candidate oil-in-water nanoemulsions and nanoparticles (chitosan). While the chitosan formulation failed to provide protection, the nanoemulsions greatly increased the protection of mice against otherwise lethal Shigella challenge (Table 11). This was especially true for the ME nanoemulsion which is similar to the human-approved AS03 adjuvant formation with BECC438 replacing the α-tocopherol adjuvant component of the formulation. While we could have included Alhydrogel (an aluminum salt) as an adjuvant/carrier, we felt that the nanoemulsions with their small size (100 to 200 nm) were a better fit for IN administration though there are not yet any such formulations for administration by this route.

The highest dose of BECC438 enhanced the adjuvanticity of the ME oil-in-water emulsion the best, however, we were somewhat concerned that too much adjuvant could overstimulate the host innate immune system and led to inflammation. To reduce the total amount of adjuvant present, we combined low doses of the self-adjuvanting L-DBF with the BECC/ME formulation instead of DBF so that we could reduce the amount of BECC present. Our previous work has demonstrated that L-DBF alone provides cross-protection against lethal challenge by different Shigella serotypes, however, this required a dose of at least 25 μg. When BECC438 was used together with the LTA1 moiety of L-DBF, it was found to provide protection at a much lower dose (Table 12), however, it was also found that the L-DBF needed for protection can be reduced to 1 μg. Neither the 10 μg or 1 μg L-DBF doses can elicit protection on their own. The inclusion of BECC438 with ME to these L-DBF doses caused the Vaccine Efficacy (VE) to increase significantly from 10% to 90%-100%. Interestingly, the BECC438 and L-DBF formulated with ME can even be reduced to 1 μg and 0.5 μg, respectively, and still provide a VE of 60% with a prime-boost-boost regimen and 50% VE with a prime-boost regimen (Table 13), however, protection dropped off sharply for a prime vaccination without any booster vaccinations.

Early work on Shigella vaccine development mainly focused on antibodies, particularly mucosal neutralizing IgA which directly prevents the bacteria from invading mucosal epithelial cells. It has been proposed that mucosal IgA levels correlated with the vaccine effects. Although antibodies are important for pathogen clearance, the data presented here indicate that T cell responses raised during vaccination are also important and it has been reported that induction of IL-17 and IFN-γ responses in response to Shigella infection also have a role in pathogen clearance and protection of the host. Our studies indicated that host protection against a lethal S. flexneri challenge is dependent upon strong IFN-γ and IL-17 responses in a mouse model. IFN-γ is an important cytokine related to the Th1 response, while IL-17 is a major cytokine that is part of the Th17 response. Therefore, in this work we analyzed the correlation between Th1/Th17 related cytokines with the vaccine efficacy. We found that vaccine-induced IL-17 secretion was important component of protective immunity against Shigella infection, but IFN-γ was also found to be important. Such a correlation was not found for other cytokines, e.g. TNF-α.

In conclusion, our findings here indicate that a Th1/Th17 response induced by vaccines using BECC438 as an adjuvant can contribute to protection from Shigella challenge and this effect is seen at low BECC438 and antigen doses when LTA1 was also present (as a component of L-DBF). The enhanced BECC438 and L-DBF as part of a polymeric presentation with the ME nanoemulsion also indicates that such a formulation is suitable for the use in humans. Some reactivity was observed when BECC438 and LTA1 (as part of L-DBF) were both used at high doses, most likely due to there being too much adjuvant power acting upon innate immune responses. However, the presence of both adjuvants (BECC438 and LTA1) at low doses did not induce any ill effects in the mice and the ability to introduce this formulation at a mucosal site (intranasally) ensured that a proper mucosal immune response was induced that was safe and protective against lethal Shigella challenge.

### E. REFERENCES

Allen H, Mitchell H D, Simms I, Baker K S, Foster K, Hughes G, Dallman T J, Jenkins C. 2021. Evidence for re-infection and persistent carriage of Shigella species in adult males reporting domestically acquired infection in England. Clin Microbiol Infect 27:126 e7-126 e13.
Anam K, Endharti A T, Poeranto S, Sujuti H, Hidayati D Y N, Prawiro S R. 2022. Shigella flexneri vaccine development: Oral administration of peptides derived from the 49.8 kDa pili protein subunit activates the intestinal immune response in mice. Vet World 15:281-287.
Anderson M, Sansonetti P J, Marteyn B S. Shigella Diversity and Changing Landscape: Insights for the Twenty-First Century. Front Cell Infect Microbiol. 2016; 6:45.
Arnold S L M. Target Product Profile and Development Path for Shigellosis Treatment with Antibacterials. ACS Infect Dis. 2021; 7(5):948-58.
Baker K S, Dallman T J, Field N, Childs T, Mitchell H, Day M, Weill F X, Lefevre S, Tourdjman M, Hughes G, Jenkins C, Thomson N. 2018. Genomic epidemiology of Shigella in the United Kingdom shows transmission of pathogen sublineages and determinants of antimicrobial resistance. Sci Rep 8:7389.
Baker K S, Dallman T J, Field N, Childs T, Mitchell H, Day M, Weill F X, Lefevre S, Tourdjman M, Hughes G, Jenkins C, Thomson N. 2018. Horizontal antimicrobial resistance transfer drives epidemics of multiple Shigella species. Nat Commun 9:1462.
Barry E M, Pasetti M F, Sztein M B, Fasano A, Kotloff K L, Levine M M. 2013. Progress and pitfalls in Shigella vaccine research. Nat Rev Gastroenterol Hepatol doi:10.1038/nrgastro.2013.12.
Bengtsson R J, Simpkin A J, Pulford C V, Low R, Rasko D A, Rigden D J, Hall N, Barry E M, Tennant S M, Baker K S. 2022. Pathogenomic analyses of Shigella isolates inform factors limiting shigellosis prevention and control across LMICs. Nat Microbiol 7:251-261.
Bento D, Staats H F, Goncalves T, Borges O. Development of a novel adjuvanted nasal vaccine: C48/80 associated with chitosan nanoparticles as a path to enhance mucosal immunity. Eur J Pharm Biopharm. 2015; 93:149-64.
Clements J D, Norton E B. The Mucosal Vaccine Adjuvant LT (R192G/L211A) or dmLT. mSphere. 2018; 3(4).
Connor T R, Barker C R, Baker K S, Weill F X, Talukder K A, Smith A M, Baker S, Gouali M, Pham Thanh D, Jahan Azmi I, Dias da Silveira W, Semmler T, Wieler L H, Jenkins C, Cravioto A, Faruque S M, Parkhill J, Wook Kim D, Keddy K H, Thomson N R. 2015. Species-wide whole genome sequencing reveals historical global spread and recent local persistence in Shigella flexneri. Elife 4:e07335.
Corthesy B, Bioley G. Lipid-Based Particles: Versatile Delivery Systems for Mucosal Vaccination against Infection. Front Immunol. 2018; 9:431.
Cossette B, Kelly S H, Collier J H. Intranasal Subunit Vaccination Strategies Employing Nanomaterials and Biomaterials. ACS Biomater Sci Eng. 2021; 7(5):1765-79.
Das S, Howlader D R, Lu T, Whittier S K, Hu G, Sharma S, et al. Immunogenicity and protective efficacy of nanoparticle formulations of L-SseB against Salmonella infection. Frontiers in Immunology. 2023; 14.
Espina M, Olive A J, Kenjale R, Moore D S, Ausar S F, Kaminski R W, Oaks E V, Middaugh C R, Picking W D, Picking W L. 2006. IpaD Localizes to the Tip of the Type III Secretion System Needle of Shigella flexneri. Infect Immun 74:4391-400.
Gan Q, Wang T. Chitosan nanoparticle as protein delivery carrier—systematic examination of fabrication conditions for efficient loading and release. Colloids Surf B Biointerfaces. 2007; 59(1):24-34.
Gregg K A, Harberts E, Gardner F M, Pelletier M R, Cayatte C, Yu L, et al. A lipid A-based TLR4 mimetic effectively adjuvants a Yersinia pestis rF-V1 subunit vaccine in a murine challenge model. Vaccine. 2018; 36(28):4023-31.
Haupt R E, Harberts E M, Kitz R J, Strohmeier S, Krammer F, Ernst R K, et al. Novel TLR4 adjuvant elicits protection against homologous and heterologous Influenza A infection. Vaccine. 2021; 39(36):5205-13.
Hawkey J, Paranagama K, Baker K S, Bengtsson R J, Weill F X, Thomson N R, Baker S, Cerdeira L, Iqbal Z, Hunt M, Ingle D J, Dallman T J, Jenkins C, Williamson D A, Holt K E. 2021. Global population structure and genotyping framework for genomic surveillance of the major dysentery pathogen, Shigella sonnei. Nat Commun 12:2684.
Heine S J, Diaz-McNair J, Andar A U, Drachenberg C B, van de Verg L, Walker R, et al. Intradermal Delivery of Shigella IpaB and IpaD Type III Secretion Proteins: Kinetics of Cell Recruitment and Antigen Uptake, Mucosal and Systemic Immunity, and Protection across Serotypes. J Immunol. 2014; 192(4): 1630-40.
Hosangadi D, Smith P G, Giersing B K. 2019. Considerations for using ETEC and Shigella disease burden estimates to guide vaccine development strategy. Vaccine 37:7372-7380.
Hosangadi D, Smith P G, Giersing B K. Considerations for using ETEC and Shigella disease burden estimates to guide vaccine development strategy. Vaccine. 2019; 37(50):7372-80.
Howlader D R, Das S, Lu T, Hu G, Varisco D J, Dietz Z K, et al. Effect of Two Unique Nanoparticle Formulations on the Efficacy of a Broadly Protective Vaccine Against Pseudomonas Aeruginosa. Front Pharmacol. 2021; 12:706157.
Iyer V, Cayatte C, Guzman B, Schneider-Ohrum K, Matuszak R, Snell A, et al. Impact of formulation and particle size on stability and immunogenicity of oil-in-water emulsion adjuvants. Hum Vaccin Immunother. 2015; 11(7):1853-64.
Jehl S P, Doling A M, Giddings K S, Phalipon A, Sansonetti P J, Goldberg M B, Starnbach M N. 2011. Antigen-specific CD8(+) T cells fail to respond to Shigella flexneri. Infect Immun 79:2021-30.
Khalil I A, Troeger C, Blacker B F, Rao P C, Brown A, Atherly D E, Brewer T G, Engmann C M, Houpt E R, Kang G, Kotloff K L, Levine M M, Luby S P, MacLennan C A, Pan W K, Pavlinac P B, Platts-Mills J A, Qadri F, Riddle M S, Ryan E T, Shoultz D A, Steele A D, Walson J L, Sanders J W, Mokdad A H, Murray C J L, Hay S I, Reiner R C, Jr. 2018. Morbidity and mortality due to shigella and enterotoxigenic Escherichia coli diarrhoea: the Global Burden of Disease Study 1990-2016. Lancet Infect Dis 18:1229-1240.
Kolling G, Wu M, Guerrant R L. 2012. Enteric pathogens through life stages. Front Cell Infect Microbiol 2:114.
Kotloff K L, Nataro J P, Blackwelder W C, Nasrin D, Farag T H, Panchalingam S, et al. Burden and aetiology of diarrhoeal disease in infants and young children in developing countries (the Global Enteric Multicenter Study, GEMS): a prospective, case-control study. Lancet. 2013; 382(9888):209-22.
Kotloff K L, Riddle M S, Platts-Mills J A, Pavlinac P, Zaidi A K M. 2018. Shigellosis. Lancet 391:801-812.
Kotloff K L, Winickoff J P, Ivanoff B, Clemens J D, Swerdlow D L, Sansonetti P J, et al. Global burden of Shigella infections: implications for vaccine development and implementation of control strategies. Bull World Health Organ. 1999; 77(8):651-66.
Kotloff K L. The Burden and Etiology of Diarrheal Illness in Developing Countries. Pediatr Clin North Am. 2017; 64(4): 799-814.
Lewis D J, Huo Z, Barnett S, Kromann I, Giemza R, Galiza E, et al. Transient facial nerve paralysis (Bell's palsy) following intranasal delivery of a genetically detoxified mutant of Escherichia coli heat labile toxin. PLOS One. 2009; 4(9):e6999.
Liu X, Wang S, Sendi L, Caulfield M J. 2004. High-throughput imaging of bacterial colonies grown on filter plates with application to serum bactericidal assays. J Immunol Methods 292:187-93. Livio S, Strockbine N A, Panchalingam S, Tennant S M, Barry E M, Marohn M E, Antonio M, Hossain A, Mandomando I, Ochieng J B, Oundo J O, Qureshi S, Ramamurthy T, Tamboura B, Adegbola R A, Hossain M J, Saha D, Sen S, Faruque A S, Alonso P L, Breiman R F, Zaidi A K, Sur D, Sow S O, Berkeley L Y, O'Reilly C E, Mintz E D, Biswas K, Cohen D, Farag T H, Nasrin D, Wu Y, Blackwelder W C, Kotloff K L, Nataro J P, Levine M M. 2014. Shigella isolates from the global enteric multicenter study inform vaccine development. Clin Infect Dis 59:933-41.
Lu T, Das, S., Howlader, D. R., Zheng, Q, Siva Sai Kumar, R., Whittier, S. K., Picking, W. D. and Picking W. L. 2021. L-DBF elicits cross protection against different serotypes of Shigella spp. Front Trop Dis doi:doi.org/10.3389/fitd.2021.729731
Lu T, Das, S., Howlader, D. R., Zheng, Q, Siva Sai Kumar, R., Whittier, S. K., Picking, W. D. and Picking W. L. L-DBF elicits cross protection against different serotypes of Shigella spp. Front Trop Dis. 2021.
Maglione P J, Chan J. 2009. How B cells shape the immune response against Mycobacterium tuberculosis. Eur J Immunol 39:676-86.
Mani S, Wierzba T, Walker R I. Status of vaccine research and development for Shigella. Vaccine. 2016; 34(26):2887-94.
Marasini N, Skwarczynski M, Toth I. Intranasal delivery of nanoparticle-based vaccines. Ther Deliv. 2017; 8(3):151-67.
Martinez-Becerra F J, Chen X, Dickenson N E, Choudhari S P, Harrison K, Clements J D, Picking W D, Van De Verg L L, Walker R I, Picking W L. 2013. Characterization of a novel fusion protein from IpaB and IpaD of Shigella spp. and its potential as a pan-Shigella vaccine. Infect Immun 81:4470-7.
Martinez-Becerra F J, Kissmann J M, Diaz-McNair J, Choudhari S P, Quick A M, Mellado-Sanchez G, Clements J D, Pasetti M F, Picking W L. 2012. Broadly protective Shigella vaccine based on type III secretion apparatus proteins. Infect Immun 80:1222-31.
Martinez-Becerra F J, Scobey M, Harrison K, Choudhari S P, Quick A M, Joshi S B, et al. Parenteral immunization with IpaB/IpaD protects mice against lethal pulmonary infection by Shigella. Vaccine. 2013; 31(24):2667-72.
Mobeen H, Safdar M, Fatima A, Afzal S, Zaman H, Mehdi Z. Emerging applications of nanotechnology in context to immunology: A comprehensive review. Front Bioeng Biotechnol. 2022; 10:1024871.
Muthuramalingam M, Whittier, S. K., Picking, W. L. and Picking, W. D. 2021. The Shigella Type III Secretion System: An Overview from Top to Bottom. Microorganisms 9:451.
Muthuramalingam M, Whittier, S. K., Picking, W. L. and Picking, W. D. The Shigella Type III Secretion System: An Overview from Top to Bottom. Microorganisms. 2021; 9(2):451.
Namiot E D, Sokolov A V, Chubarev V N, Tarasov V V, Schioth H B. Nanoparticles in Clinical Trials: Analysis of Clinical Trials, FDA Approvals and Use for COVID-19 Vaccines. Int J Mol Sci. 2023; 24(1).
Ndungo E, Pasetti M F. 2020. Functional antibodies as immunological endpoints to evaluate protective immunity against Shigella. Human Vaccines & Immunotherapeutics 16:197-205.
Norton E B, Lawson L B, Mahdi Z, Freytag L C, Clements J D. 2012. The A Subunit of Escherichia coli Heat-Labile Enterotoxin Functions as a Mucosal Adjuvant and Promotes IgG2a, IgA, and Th17 Responses to Vaccine Antigens. Infection and Immunity 80:2426-2435.
Olive A J, Kenjale R, Espina M, Moore D S, Picking W L, Picking W D. 2007. Bile salts stimulate recruitment of IpaB to the Shigella flexneri surface, where it colocalizes with IpaD at the tip of the type III secretion needle. Infect Immun 75:2626-9.
Pore D, Chakrabarti M K. 2013. Outer membrane protein A (OmpA) from Shigella flexneri 2a: a promising subunit vaccine candidate. Vaccine 31:3644-50.
Rew V, Mook P, Trienekens S, Baker K S, Dallman T J, Jenkins C, Crook P D, Thomson N R. 2018. Whole-genome sequencing revealed concurrent outbreaks of shigellosis in the English Orthodox Jewish Community caused by multiple importations of Shigella sonnei from Israel. Microb Genom 4.
Salgado-Pabon W, Celli S, Arena E T, Nothelfer K, Roux P, Sellge G, Frigimelica E, Bousso P, Sansonetti P J, Phalipon A. 2013. Shigella impairs T lymphocyte dynamics in vivo. Proc Natl Acad Sci USA 110:4458-63.
Salgado-Pabon W, Konradt C, Sansonetti P J, Phalipon A. 2014. New insights into the crosstalk between Shigella and T lymphocytes. Trends in Microbiology 22:192-198.
Samassa F, Ferrari M L, Husson J, Mikhailova A, Porat Z, Sidaner F, Brunner K, Teo T H, Frigimelica E, Tinevez J Y, Sansonetti P J, Thoulouze M I, Phalipon A. 2020. Shigella impairs human T lymphocyte responsiveness by hijacking actin cytoskeleton dynamics and T cell receptor vesicular trafficking. Cell Microbiol 22:e13166.
Sellge G, Magalhaes J G, Konradt C, Fritz J H, Salgado-Pabon W, Eberl G, Bandeira A, Di Santo J P, Sansonetti P J, Phalipon A. 2010. Th17 Cells Are the Dominant T Cell Subtype Primed by Shigella flexneri Mediating Protective Immunity. Journal of Immunology 184:2076-2085.
Sellge G, Magalhaes J G, Konradt C, Fritz J H, Salgado-Pabon W, Eberl G, et al. Th17 cells are the dominant T cell subtype primed by Shigella flexneri mediating protective immunity. J Immunol. 2010; 184(4):2076-85.
Sevil Domenech V E, Panthel K, Meinel K M, Winter S E, Russmann H. 2007. Pre-existing anti-Salmonella vector immunity prevents the development of protective antigen-specific CD8 T-cell frequencies against murine listeriosis. Microbes and Infection 9:1447-1453.
Tallant A, Porter C K, Putnam S D, Tribble D R, Hooper T I, Riddle M S. 2014. Relative cost-effectiveness of a norovirus vaccine in the deployed military setting compared to a vaccine against Campylobacter sp., ETEC, and Shigella sp. Vaccine 32:5156-62.
Turner J S, Kim W, Kalaidina E, Goss C W, Rauseo A M, Schmitz A J, Hansen L, Haile A, Klebert M K, Pusic I, O'Halloran J A, Presti R M, Ellebedy A H. 2021. SARS-COV-2 infection induces long-lived bone marrow plasma cells in humans. Nature 595:421-425.
van de Verg L L, Mallett C P, Collins H H, Larsen T, Hammack C, Hale T L. 1995. Antibody and cytokine responses in a mouse pulmonary model of Shigella flexneri serotype 2a infection. Infect Immun 63:1947-54.
Wagner AaW, B. Vaccines to Prevent Infectious Diseases in the Older Population: Immunological Challenges and Future Perspectives. Frontiers in Immunology. 2020; 11.
Way S S, Borczuk A C, Dominitz R, Goldberg M B. An essential role for gamma interferon in innate resistance to Shigella flexneri infection. Infect Immun. 1998; 66(4):1342-8.
Williams P C M, Berkley J A. Guidelines for the treatment of dysentery (shigellosis): a systematic review of the evidence. Paediatr Int Child Health. 2018; 38(sup1):S50-S65.
Xie C B, Jane-Wit D, Pober J S. 2020. Complement Membrane Attack Complex: New Roles, Mechanisms of Action, and Therapeutic Targets. Am J Pathol 190:1138-1150.

F. Sequences
IpaD amino acid sequence
SEQ ID NO: 1
MNITTLTNSISTSSFSPNNTNGSSTETVNSDIKTTTSSHPVSSLTMLNDTLHNIRTTNQA
LKKELSQKTLTKTSLEEIALHSSQISMDVNKSAQLLDILSRNEYPINKDARELLHSAPK
EAELDGDQMISHRELWAKIANSINDINEQYLKVYEHAVSSYTQMYQDFSAVLSSLAG
WISPGGNDGNSVKLQVNSLKKALEELKEKYKDKPLYPANNTVSQEQANKWLTELG
GTIGKVSQKNGGYVVSINMTPIDNMLKSLDNLGGNGEVVLDNAKYQAWNAGFSAE
DETMKNNLQTLVQKYSNANSIFDNLVKVLSSTISSCTDTDKLFLHF
IpaD nucleic acid sequence
SEQ ID NO: 2
ATGAATATAACAACTCTGACTAATAGTATTTCCACCTCATCATTCAGTCCAAACA
ATACCAACGGTTCATCAACCGAAACAGTTAATTCTGATATAAAAACAACGACCA
GTTCTCATCCTGTAAGTTCCCTTACTATGCTCAACGACACCCTTCATAATATCAGA
ACAACAAATCAGGCATTAAAGAAAGAGCTTTCACAAAAAACGTTGACTAAAACA
TCGCTAGAAGAAATAGCATTACATTCATCTCAGATTAGCATGGATGTAAATAAAT
CCGCTCAACTATTGGATATTCTTTCCAGGAACGAATATCCAATTAATAAAGACGC
AAGAGAATTATTACATTCAGCCCCGAAAGAAGCCGAGCTTGATGGAGATCAAAT
GATATCTCATAGAGAACTGTGGGCTAAAATTGCAAACTCCATCAATGATATTAAT
GAACAGTATCTGAAAGTATATGAACATGCCGTTAGTTCATATACTCAAATGTATC
AAGATTTTAGCGCTGTTCTTTCCAGTCTTGCCGGCTGGATCTCTCCCGGAGGTAA
CGACGGAAACTCCGTGAAATTACAAGTCAACTCGCTTAAAAAGGCATTGGAAGA
ACTCAAGGAAAAATATAAAGATAAACCGCTATATCCAGCAAATAATACTGTTAG
TCAGGAACAAGCAAATAAATGGCTTACAGAATTAGGTGGAACAATCGGCAAGGT
ATCTCAAAAAAACGGGGGATATGTTGTCAGTATAAACATGACCCCAATAGACAA
TATGTTAAAAAGCTTAGATAATCTAGGTGGAAATGGCGAGGTTGTGCTAGATAA
TGCAAAATATCAGGCATGGAATGCCGGATTCTCTGCCGAAGATGAAACAATGAA
AAATAATCTTCAAACTTTAGTTCAAAAATACAGTAATGCCAATAGTATTTTTGAT
AATTTAGTAAAGGTTTTGAGTAGTACAATAAGCTCATGTACAGATACAGATAAA
CTTTTTCTCCATTTCTGA
IpaB amino acid sequence
SEQ ID NO: 3
MHNVSTTTTGFPLAKILTSTELGDNTIQAANDAANKLFSLTIADLTANQNINTTNAHS
TSNILIPELKAPKSLNASSQLTLLIGNLIQILGEKSLTALTNKITAWKSQQQARQQKNL
EFSDKINTLLSETEGLTRDYEKQINKLKNADSKIKDLENKINQIQTRLSNLDPESPEKK
KLSREEIQLTIKKDAAVKDRTLIEQKTLSIHSKLTDKSMQLEKEIDSFSAFSNTASAEQ
LSTQQKSLTGLASVTQLMATFIQLVGKNNEESLKNDLALFQSLQESRKTEMERKSDE
YAAEVRKAEELNRVMGCVGKILGALLTIVSVVAAAFSGGASLALAAVGLALMVTD
AIVQAATGNSFMEQALNPIMKAVIEPLIKLLSDAFTKMLEGLGVDSKKAKMIGSILGA
IAGALVLVAAVVLVATVGKQAAAKLAENIGKIIGKTLTDLIPKFLKNFSSQLDDLITN
AVARLNKFLGAAGDEVISKQIISTHLNQAVLLGESVNSATQAGGSVASAVFQNSAST
NLADLTLSKYQVEQLSKYISEAIEKFGQLQEVIADLLASMSNSQANRTDVAKAILQQ
TTA
IpaB nucleic acid sequence
SEQ ID NO: 4
ATGCATAATGTAAGCACCACAACCACTGGTTTTCCTCTTGCCAAAATATTGACTT
CCACTGAGCTTGGAGACAATACTATCCAAGCTGCAAATGATGCAGCTAACAAAT
TATTTTCTCTTACAATTGCTGATCTTACTGCTAACCAAAATATTAATACAACTAAT
GCACACTCAACTTCAAATATATTAATCCCTGAACTTAAAGCACCAAAGTCATTAA
ATGCAAGTTCCCAACTAACGCTTTTAATTGGAAACCTTATTCAAATACTCGGTGA
AAAATCTTTAACTGCATTAACAAATAAAATTACTGCTTGGAAGTCCCAGCAACAG
GCAAGACAGCAAAAAAACCTAGAATTCTCCGATAAAATTAACACTCTTCTATCTG
AAACTGAAGGACTAACCAGAGACTATGAAAAACAAATTAATAAACTAAAAAAC
GCAGATTCTAAAATAAAAGACCTAGAAAATAAAATTAACCAAATTCAAACAAGA
TTATCGAACCTCGATCCAGAGTCACCAGAAAAGAAAAAATTAAGCCGGGAAGAA
ATACAACTCACTATCAAAAAAGACGCAGCAGTTAAAGACAGGACATTGATTGAG
CAGAAAACCCTGTCAATTCATAGCAAACTTACAGATAAATCAATGCAACTCGAA
AAAGAAATAGACTCTTTTTCTGCATTTTCAAACACAGCATCTGCTGAACAGCTAT
CAACCCAGCAGAAATCATTAACCGGACTTGCCAGTGTTACTCAATTGATGGCAAC
CTTTATTCAACTAGTTGGAAAAAATAATGAAGAATCTTTAAAAAATGATCTGGCT
CTATTCCAGTCTCTCCAAGAATCAAGAAAAACTGAAATGGAGAGAAAATCTGAT
GAGTATGCTGCTGAAGTACGTAAAGCAGAAGAACTCAACAGAGTAATGGGTTGT
GTTGGGAAAATACTTGGGGCACTTTTAACTATCGTTAGTGTTGTTGCAGCAGCTT
TTTCTGGAGGAGCCTCTCTAGCACTGGCAGCTGTTGGTTTAGCTCTTATGGTTACG
GATGCTATAGTACAAGCAGCGACCGGCAATTCCTTCATGGAACAAGCCCTGAAT
CCGATCATGAAAGCAGTCATTGAACCCTTAATCAAACTCCTTTCAGATGCATTTA
CAAAAATGCTCGAAGGCTTGGGCGTCGACTCGAAAAAAGCCAAAATGATTGGCT
CTATTCTGGGGGCAATCGCAGGCGCTCTTGTCCTAGTTGCAGCAGTCGTTCTCGT
AGCCACTGTTGGTAAACAGGCAGCAGCAAAACTTGCAGAAAATATTGGCAAAAT
AATAGGTAAAACCCTCACAGACCTTATACCAAAGTTTCTCAAGAATTTTTCTTCT
CAACTGGACGATTTAATCACTAATGCTGTTGCCAGATTAAATAAATTTCTTGGTG
CAGCGGGTGATGAAGTAATATCCAAACAAATTATTTCCACCCATTTAAACCAAGC
AGTTTTATTAGGAGAAAGTGTTAACTCTGCCACACAAGCGGGAGGAAGTGTCGC
TTCTGCTGTTTTCCAGAACAGCGCGTCGACAAATCTAGCAGACCTGACATTATCG
AAATATCAAGTTGAACAACTGTCAAAATATATCAGTGAAGCAATAGAAAAATTC
GGCCAATTGCAGGAAGTAATTGCAGATCTATTAGCCTCAATGTCCAACTCTCAGG
CTAATAGAACTGATGTTGCAAAAGCAATTTTGCAACAAACTACTGCTTGA
dmLT eltA (LTa)nucleic acid sequence
SEQ ID NO: 5
atgattgaca tcatgttgca tataggttag ataaaacaag tggttatctt tccggattgt
cttcttgtat gatatataag ttttcctcga tgaaaaatat aactttcatt ttttttattt
tattagcatc gccattatat gcaaatggcg acagattata ccgtgctgac tctagacccc
cagatgaaat aaaacgtttc cggagtctta tgcccagagg taatgagtac ttcgatagag
gaactcaaat gaatattaat ctttatgatc acgcgagagg aacacaaacc ggctttgtca
gatatgatga cggatatgtt tccacttctc ttagtttgag aagtgctcac ttagcaggac
agtatatatt atcaggatat tcacttacta tatatatcgt tatagcaaat atgtttaatg
ttaatgatgt aattagcgta tacagccctc acccatatga acaggaggtt tctgcgttag
gtggaatacc atattctcag atatatggat ggtatcgtgt taattttggt gtgattgatg
aacgattaca tcgtaacagg gaatatagag accggtatta cagaaatctg aatatagctc
cggcagagga tggttacaga ttagcaggtt tcccaccgga tcaccaagct tggagagaag
aaccctggat tcatcatgca ccacaaggtt gtggagattc atcaGgaaca atcacaggtg
atacttgtaa tgaggagacc cagaatctga gcacaatata tGCcagggaa tatcaatcaa
aagttaagag gcagatattt tcagactatc agtcagaggt tgacatatat aacagaattc
gggatgaatt atgaataaag taaaatgt
eltB (LTb) nucleic acid sequence
SEQ ID NO: 6
gttgacatat ataacagaat tcgggatgaa ttatgaataa agtaaaatgt tatgttttat
ttacggcgtt actatcctct ctatatgcac acggagctcc ccagactatt acagaactat
gttcggaata tcgcaacaca caaatatata cgataaatga caagatacta tcatatacgg
aatcgatggc aggcaaaaga gaaatggtta tcattacatt taagagcggc gaaacatttc
aggtcgaagt cccgggcagt caacatatag actcccagaa aaaagccatt gaaaggatga
aggacacatt aagaatcaca tatctgaccg agaccaaaat tgataaatta tgtgtatgga
ataataaaac ccccaattca attgcggcaa tcagtatgaa aaactagttt gctttaaaag
catgtctaat gctaggaacc tatataacaa ctactgtact tatactaatg agccttatgc
tgcatttgaa aaggcggtag aggaggcaat accgatcctt aaactgtaac actataacag
cttccactac agggagctgt tatagcacac agaaaaaact aagctaggct ggaggggcaa
gctt
His-BcrH1 chaperone with histidine tag nucleotide sequence
SEQ ID NO: 7
ATGGGCAGCAGCCATCACCATCATCACCACAGCCAGGATCCGATGCCAAA
GTCAGCCGAGCAGGGCGGCTCCCCGGCGTCAGCTTCGCATGAGGCGTTGCGCCA
TATTCTCGACGCAGGCGCTTCGATGGGCAGCTTGCAGGGGTTGGACGAGGTGCA
ACAGCAGGCGTTGTACGCGATCGCTCATGGCGCCTACGAACAGGGCCGCTATGC
CGACGCGTTGAAAATGTTCTGCCTGCTGGTCGCGTGCGATCCGCTGGAAGCCCGT
TATCTGCTGGCCCTGGGCGCCGCGGCCCAGGAGCTGGGGCTGTACGAGCATGCC
TTGCAGCAATACGCGGCCGCGGCGGCTTTGCAGTTGGACTCCCCCAGGCCCCTGT
TGCATGGCGCCGAGTGCCTGTATGCGTTGGGTCGTCGCCGCGACGCCCTGGATAC
GCTCGACATGGTGCTTGAGTTGTGCGGGTCGCCGGAGCATGCGGCCCTGCGCGA
ACGGGCCGAGTCGCTGCGCAGGAGCTATGCACGTGCCGACTGAAAGCTT
His-BcrH1 with histidine tag chaperone amino acid sequence
SEQ ID NO: 8
MGSSHHHHHHSQDPMPKSAEQGGSPASASHEALRHILDAGASMGSLQGLDE
VQQQALYAIAHGAYEQGRYADALKMFCLLVACDPLEARYLLALGAAAQELGLYEH
ALQQYAAAAALQLDSPRPLLHGAECLYALGRRRDALDTLDMVLELCGSPEHAALRE
RAESLRRSYARAD
BcrH1 amino acid sequence
SEQ ID NO: 9
MPKSAEQGGSPASASHEALRHILDAGASMGSLQGLDEVQQQALYAIAHGAY
EQGRYADALKMFCLLVACDPLEARYLLALGAAAQELGLYEHALQQYAAAAALQLD
SPRPLLHGAECLYALGRRRDALDTLDMVLELCGSPEHAALRERAESLRRSYARAD
IpgC Chaperone of DBF nucleic acidsequence
SEQ ID NO: 10
CCatgggcagcagccatcatcatcatcatcacagcagcggcctggtgccgcgcggcagccatatg
ctcgagatgtctttaaatatcaccgaaaatgaaagcatctctactgcagtaattgatgcaattaa
ctctggcgctacactgaaagatattaatgcaattcctgatgatatgatggatgacatttattcat
atgcttatgacttttacaacaaaggaagaatagaggaagctgaagttttcttcaggtttttatgt
atatacgacttttacaatatagactacattatgggactcgcagctatttatcagataaaagaaca
gttccaacaagcagcagacctttatgctgtcgcttttgcattaggaaaaaatgactatacaccag
tattccatactggacaatgccagcttcggttgaaagcccccttaaaagctaaagagtgcttcgaa
ctcgtaattcaacacagcaatgatgaaaaattaaaaataaaagcacaatcatacttggacgcaat
tcaggatatcaaggagtagGATCC
IpgC Chaperone of DBF Amino Acid sequence
SEQ ID NO: 11
MSLNITENESISTAVIDAINSGATLKDINAIPDDMMDDIYSYAYDFYNKGRIEE
AEVFFRFLCIYDFYNVDYIMGLAAIYQIKEQFQQAADLYAVAFALGKNDYTPVFHTG
QCQLRLKAPLKAKECFELVIQHSNDEKLKIKAQSYLDAIQDIKE
LTA1 nucleic acid sequence
SEQ ID NO: 12
CATAtggacaatggcgatcgtttataccgtgccgactcgcgtcccccagatgagattaaacgtag
cggtgggttaatgccacgtgggcacaatgagtattttgaccgtggaacacagatgaacattaacc
tttacgatcatgcccgtgggacccagaccgggtttgtccgttatgatgacgggtatgttagtacg
agtttgtccttacgctccgcacaccttgcgggacaaagtattttatcaggctacagcacatatta
catttatgtgatcgccactgccccaaacatgttcaatgtgaacgatgtgttgggggtttacagcc
cccatccatatgaacaagaagtctcggcccttggggggatcccatatagccagatttatggttgg
taccgcgtaaattttggtgtgattgatgaacgtttgcatcgtaaccgtgaataccgcgatcgcta
ctaccgtaacttgaacattgcacctgccgaggacggctatcgtttagcgggattcccacccgatc
atcaggcgtggcgtgaggaaccgtggatccatcacgcccctcaggggtgcgggaacagtagtcgc
LTA1 amino acid sequence
SEQ ID NO: 13
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSR
GSAAS Linker amino acid sequence
SEQ ID NO: 14
GSAAS
LTA1-GSAAS-DBF (IpaD-LE-IpaB) nucleic acid sequence
SEQ ID NO: 15
CATAtggacaatggcgatcgtttataccgtgccgactcgcgtcccccagatgagattaaacgtag
cggtgggttaatgccacgtgggcacaatgagtattttgaccgtggaacacagatgaacattaacc
tttacgatcatgcccgtgggacccagaccgggtttgtccgttatgatgacgggtatgttagtacg
agtttgtccttacgctccgcacaccttgcgggacaaagtattttatcaggctacagcacatatta
catttatgtgatcgccactgccccaaacatgttcaatgtgaacgatgtgttgggggtttacagcc
cccatccatatgaacaagaagtctcggcccttggggggatcccatatagccagatttatggttgg
taccgcgtaaattttggtgtgattgatgaacgtttgcatcgtaaccgtgaataccgcgatcgcta
ctaccgtaacttgaacattgcacctgccgaggacggctatcgtttagcgggattcccacccgatc
atcaggcgtggcgtgaggaaccgtggatccatcacgcccctcaggggtgcgggaacagtagtcgc
gggtccgcggcatccatgaatat aacaactctg actaatagta tttccacctc
atcattcagt ccaaacaata ccaacggttc atcaaccgaa acagttaatt ctgatataaa
aacaacgacc agttctcatc ctgtaagttc ccttactatg ctcaacgaca cccttcataa
tatcagaaca acaaatcagg cattaaagaa agagctttca caaaaaacgt tgactaaaac
atcgctagaa gaaatagcat tacattcatc tcagattagc atggatgtaa ataaatccgc
tcaactattg gatattcttt ccaggaacga atatccaatt aataaagacg caagagaatt
attacattca gccccgaaag aagccgagct tgatggagat caaatgatat ctcatagaga
actgtgggct aaaattgcaa actccatcaa tgatattaat gaacagtatc tgaaagtata
tgaacatgcc gttagttcat atactcaaat gtatcaagat tttagcgctg ttctttccag
tcttgccggc tggatctctc ccggaggtaa cgacggaaac tccgtgaaat tacaagtcaa
ctcgcttaaa aaggcattgg aagaactcaa ggaaaaatat aaagataaac cgctatatcc
agcaaataat actgttagtc aggaacaagc aaataaatgg cttacagaat taggtggaac
aatcggcaag gtatctcaaa aaaacggggg atatgttgtc agtataaaca tgaccccaat
agacaatatg ttaaaaagct tagataatct aggtggaaat ggcgaggttg tgctagataa
tgcaaaatat caggcatgga atgccggatt ctctgccgaa gatgaaacaa tgaaaaataa
tcttcaaact ttagttcaaa aatacagtaa tgccaatagt atttttgata atttagtaaa
ggttttgagt agtacaataa gctcatgtac agatacagat aaactttttc tccatttc
CTCGAG atgcataatgta agcaccacaa ccactggttt tcctcttgcc aaaatattga
cttccactgagcttggagac aatactatcc aagctgcaaa tgatgcagct aacaaattat
tttctcttacaattgctgat cttactgcta accaaaatat taatacaact aatgcacact
caacttcaaatatattaatc cctgaactta aagcaccaaa gtcattaaat gcaagttccc
aactaacgcttttaattgga aaccttattc aaatactcgg tgaaaaatct ttaactgcat
taacaaataaaattactgct tggaagtccc agcaacaggc aagacagcaa aaaaacctag
aattctccgataaaattaac actcttctat ctgaaactga aggactaacc agagactatg
aaaaacaaattaataaacta aaaaacgcag attctaaaat aaaagaccta gaaaataaaa
ttaaccaaattcaaacaaga ttatcgaacc tcgatccaga gtcaccagaa aagaaaaaat
taagccgggaagaaatacaa ctcactatca aaaaagacgc agcagttaaa gacaggacat
tgattgagcagaaaaccctg tcaattcata gcaaacttac agataaatca atgcaactcg
aaaaagaaatagactctttt tctgcatttt caaacacagc atctgctgaa cagctatcaa
cccagcagaaatcattaacc ggacttgcca gtgttactca attgatggca acctttattc
aactagttggaaaaaataat gaagaatctt taaaaaatga tctggctcta ttccagtctc
tccaagaatcaagaaaaact gaaatggaga gaaaatctga tgagtatgct gctgaagtac
gtaaagcagaagaactcaac agagtaatgg gttgtgttgg gaaaatactt ggggcacttt
taactatcgttagtgttgtt gcagcagctt tttctggagg agcctctcta gcactggcag
ctgttggtttagctcttatg gttacggatg ctatagtaca agcagcgacc ggcaattcct
tcatggaacaagccctgaat ccgatcatga aagcagtcat tgaaccctta atcaaactcc
tttcagatgcatttacaaaa atgctcgaag gcttgggcgt cgactcgaaa aaagccaaaa
tgattggctctattctgggg gcaatcgcag gcgctcttgt cctagttgca gcagtcgttc
tcgtagccactgttggtaaa caggcagcag caaaacttgc agaaaatatt ggcaaaataa
taggtaaaaccctcacagac cttataccaa agtttctcaa gaatttttct tctcaactgg
acgatttaatcactaatgct gttgccagat taaataaatt tcttggtgca gcgggtgatg
aagtaatatccaaacaaatt atttccaccc atttaaacca agcagtttta ttaggagaaa
gtgttaactctgccacacaa gcgggaggaa gtgtcgcttc tgctgttttc cagaacagcg
cgtcgacaaatctagcagac ctgacattat cgaaatatca agttgaacaa ctgtcaaaat
atatcagtgaagcaatagaa aaattcggcc aattgcagga agtaattgca gatctattag
cctcaatgtccaactctcag gctaatagaa ctgatgttgc aaaagcaatt ttgcaacaaa
ctactgcttga GGATCC
LTA1-GSAAS-IpaD-LE-IpaB (DBF) Amino Acid sequence
SEQ ID NO: 16
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRGSAASMNITTLTNSISTSSFSPNNINGSS
TETVNSDIKTTTSSHPSSLTMLNDTLHNIRTTNQALKKELSQKTLRNEYPINKDAREL
LHSAPKEAELDGDQMISHRELWAKIANSINDINEQYLKVYEHAVSSYTQMYQDFSA
VLSSLAGWISPGGNDGNSVKLQVNSLKKALEELKEKYKDKPLYPANNTVSQEQANK
WLTELGGTIGKVSQKNGGYVVSINMTPIDNMLKSLDNLGGNGEVVLDNAKYQAWN
GFSAEDETMKNNLQTLVQKYSNANSIFDNLVKVLSSTISSCTDTDKLFLHFLEMHNV
STTTTGFPLAKILTSTELGDNTIQAANDAANKLESLTIADLTANQNINTTNAHSTSNILI
PELKAPKSLNASSQLTLLIGNLIQILGEKSLTALTNKITAWKSQQQARQQKNLEFSDKI
NTLLSETEGLTRDYEKQINKLKNADSKIKDLENKINQIQTRLSNLDPESPEKKKLSREE
IQLTIKKDAAVKDRTLIEQKTLSIHSKLTDKSMQLEKEIDSFSAFSNTASAEQLSTQQK
SLTGLASVTQLMATFIQLVGKNNEESLKNDLALFQSLQESRKTEMERKSDEYAAEVR
KAEELNRVMGCVGKILGALLTIVSVVAAAFSGGASLALAAVGLALMVTDAIVQAAT
GNSFMEQALNPIMKAVIEPLIKLLSDAFTKMLEGLGVDSKKAKMIGSILGAIAGALVL
VAAVVLVATVGKQAAAKLAENIGKIIGKTLTDLIPKFLKNFSSQLDDLITNAVARLN
KFLGAAGDEVISKQIISTHLNQAVLLGESVNSATQAGGSVASAVFQNSASTNLADLT
LSKYQVEQLSKYISEAIEKFGQLQEVIADLLASMSNSQANRTDVAKAILQQTTA
His-PcrH (Chaperone of PaF) nucleic acid sequence
SEQ ID NO: 17
ATGGGCAGCAGCCATCACCATCATCACCACAGCCAGGATCCGATGAACCA
GCCGACCCCTTCCGACACCGACCAGCAACAGGCGCTGGAGGCCTTCCTGCGCGA
CGGCGGCACCCTGGCGATGCTTCGCGGACTCAGCGAGGACACCCTGGAGCAGCT
CTATGCGCTGGGCTTCAACCAGTACCAGGCGGGCAAGTGGGACGACGCGCAGAA
GATCTTCCAGGCACTGTGCATGCTCGACCACTACGACGCCCGCTACTTTCTCGGC
CTGGGCGCCTGCCGCCAGTCCCTCGGTCTCTATGAACAGGCCCTGCAGAGCTACA
GCTACGGCGCGCTGATGGACATCAACGAGCCGCGCTTTCCCTTCCATGCCGCCGA
GTGCCACCTGCAACTGGGTGATCTCGACGGAGCCGAGAGTGGCTTCTACTCGGCC
CGGGCCCTGGCCGCGGCACAGCCGGCGCACGAGGCCCTGGCCGCGCGTGCCGGC
GCCATGTTGGAAGCCGTAACCGCGAGAAAGGATCGAGCCTATGAATCCGATAAC
GCTTGAAAGCTT
His-PcrH (Chaperone of PaF) Amino acid sequence
SEQ ID NO: 18
MGSSHHHHHHSQDPMNQPTPSDTDQQQALEAFLRDGGTLAMLRGLSEDTLE
QLYALGFNQYQAGKWDDAQKIFQALCMLDHYDARYFLGLGACRQSLGLYEQALQS
YSYGALMDINEPRFPFHAAECHLQLGDLDGAESGFYSARALAAAQPAHEALAARAG
AMLEAVTARKDRAYESDNA-
His-SicA chaperone for S1 nucleic sequence
SEQ ID NO: 19
ATGGGCAGCAGCCATCACCATCATCACCACAGCCAGGATCCGatggactaccaga
acaacgtcagcgaagaacgtgttgcggaaatgatttgggatgccgttagtgaaggcgccacgcta
aaagacgttcatggaatccctcaagatatgatggacggtttatatgctcatgcttatgagtttta
taaccagggacgactggatgaagctgagacgttctttcgtttcttatgcatttatgatttttaca
atcccgattacaccatgggactggcggcagtatgccaactgaaaaaacaatttcagaaagcatgt
gacctttatgcagtagcgtttacgttacttaaaaatgattatcgccccgttttttttaccgggca
gtgtcaattattaatgcgtaaggcagcaaaagccagacagtgttttgaacttgtcaatgaacgta
ctgaagatgagtctctgcgggcaaaagcgttggtctatctggaggcgctaaaaacggcggagaca
gagcagcacagcgagcaggagaaggagtaaAAGCTT
His-SicA chaperone for S1 Amino acid sequence
SEQ ID NO: 20
MGSSHHHHHHSQDPMDYQNNVSEERVAEMIWDAVSEGATLKDVHGIPQDM
MDGLYAHAYEFYNQGRLDEAETFFRFLCIYDFYNPDYTMGLAAVCQLKKQFQKAC
DLYAVAFTLLKNDYRPVFFTGQCQLLMRKAAKARQCFELVNERTEDESLRAKALVY
LEALKTAETEQHSEQEKE*
SipD nucleic acid sequence
SEQ ID NO: 21
atgcttaatattcaaaattattccgcttctcctcatccggggatcgttgccgaacggccgcagac
tccctcggcgagcgagcacgtcgagactgccgtggtaccgtctaccacagaacatcgcggtacag
atatcatttcattatcgcaggcggctactaaaatccaccaggcacagcagacgctgcagtcaacg
ccaccgatctctgaagagaataatgacgagcgcacgctggcgcgccagcagttgaccagcagcct
gaatgcgctggcgaagtccggcgtgtcattatccgcagaacaaaatgagaacctgcggagcgcgt
tttctgcgccgacgtcggccttatttagcgcttcgcctatggcgcagccgagaacaaccatttct
gatgctgagatttgggatatggtttcccaaaatatatcggcgataggtgacagctatctgggcgt
ttatgaaaacgttgtcgcagtctataccgatttttatcaggccttcagtgatattctttccaaaa
tgggaggctggttattaccaggtaaggacggtaataccgttaagctagatgttacctcactcaaa
aatgatttaaacagtttagtcaataaatataatcaaataaacagtaataccgttttatttccagc
gcagtcaggcagcggcgttaaagtagccactgaagcggaagcgagacagtggctcagtgaattga
atttaccgaatagctgcctgaaatcttatggatccggttatgtcgtcaccgttgatctgacgcca
ttacaaaaaatggttcaggatattgatggtttaggcgcgccgggaaaagactcaaaactcgaaat
ggataacgccaaatatcaagcctggcagtcgggttttaaagcgcaggaagaaaatatgaaaacca
cattacagacgctgacgcaaaaatatagcaatgccaattcattgtacgacaacctggtaaaagtg
ctgagcagtacgataagtagcagcctggaaaccgccaaaagcttcctgcaagga
SipD amino acid sequence
SEQ ID NO: 22
MLNIQNYSASPHPGIVAERPQTPSASEHVETAVVPSTTEHRGTDIISLSQAATKI
HQAQQTLQSTPPISEENNDERTLARQQLTSSLNALAKSGVSLSAEQNENLRSAFSAPT
SALFSASPMAQPRTTISDAEIWDMVSQNISAIGDSYLGVYENVVAVYTDFYQAFSDIL
SKMGGWLLPGKDGNTVKLDVTSLKNDLNSLVNKYNQINSNTVLFPAQSGSGVKVA
TEAEARQWLSELNLPNSCLKSYGSGYVVTVDLTPLQKMVQDIDGLGAPGKDSKLEM
DNAKYQAWQSGFKAQEENMKTTLQTLTQKYSNANSLYDNLVKVLSSTISSSLETAK
SFLQG
SipB nucleic acid sequence
SEQ ID NO: 23
atggtaaatgacgcaagtagcattagccgtagcggatatacccaaaatccgcgcctcgctgaggc
ggcttttgaaggcgttcgtaagaacacggactttttaaaagcggcggataaagcttttaaagatg
tggtggcaacgaaagcgggcgaccttaaagccggaacaaagtccggcgagagcgctattaatacg
gtgggtctaaagccgcctacggacgccgcccgggaaaaactctccagcgaagggcaattgacatt
actgcttggcaagttaatgaccctactgggcgatgtttcgctgtctcaactggagtctcgtctgg
cggtatggcaggcgatgattgagtcacaaaaagagatggggattcaggtatcgaaagaattccag
acggctctgggagaggctcaggaggcgacggatctctatgaagccagtatcaaaaagacggatac
cgccaagagtgtttatgacgctgcgaccaaaaaactgacgcaggcgcaaaataaattgcaatcgc
tggacccggctgaccccggctatgcacaagctgaagccgcggtagaacaggccggaaaagaagcg
acagaggcgaaagaggccttagataaggccacggatgcgacggttaaagcaggcacagacgccaa
agcgaaagccgagaaagcggataacattctgaccaaattccagggaacggctaatgccgcctctc
agaatcaggtttcccagggtgagcaggataatctgtcaaatgtcgcccgcctcactatgctcatg
gccatgtttattgagattgtgggcaaaaatacggaagaaagcctgcaaaacgatcttgcgctttt
caacgccttgcaggaagggcgtcaggcggagatggaaaagaaatcggctgaattccaggaagaga
cgcgcaaagccgaggaaacgaaccgcattatgggatgtatcgggaaagtcctcggcgcgctgcta
accattgtcagcgttgtggccgctgtttttaccggtggggcgagtctggcgctggctgcggtggg
acttgcggtaatggtggccgatgaaattgtgaaggcggcgacgggagtgtcgtttattcagcagg
cgctaaacccgattatggagcatgtgctgaagccgttaatggagctgattggcaaggcgattacc
aaagcgctggaaggattaggcgtcgataagaaaacggcagagatggccggcagcattgttggtgc
gattgtcgccgctattgccatggtggcggtcattgtggtggtcgcagttgtcgggaaaggcgcgg
cggcgaaactgggtaacgcgctgagcaaaatgatgggcgaaacgattaagaagttggtgcctaac
gtgctgaaacagttggcgcaaaacggcagcaaactctttacccaggggatgcaacgtattactag
cggtctgggtaatgtgggtagcaagatgggcctgcaaacgaatgccttaagtaaagagctggtag
gtaataccctaaataaagtggcgttgggcatggaagtcacgaataccgcagcccagtcagccggt
ggtgttgccgagggcgtatttattaaaaatgccagcgaggcgcttgctgattttatgctcgcccg
ttttgccatggatcagattcagcagtggcttaaacaatccgtagaaatatttggtgaaaaccaga
aggtaacggcggaactgcaaaaagccatgtcttctgcggtacagcaaaatgcggatgcttcgcgt
tttattctgcgccagagtcgcgcataa
SipB amino acid sequence
SEQ ID NO: 24
MVNDASSISRSGYTQNPRLAEAAFEGVRKNTDFLKAADKAFKDVVATKAGD
LKAGTKSGESAINTVGLKPPTDAAREKLSSEGQLTLLLGKLMTLLGDVSLSQLESRL
AVWQAMIESQKEMGIQVSKEFQTALGEAQEATDLYEASIKKTDTAKSVYDAATKKL
TQAQNKLQSLDPADPGYAQAEAAVEQAGKEATEAKEALDKATDATVKAGTDAKA
KAEKADNILTKFQGTANAASQNQVSQGEQDNLSNVARLTMLMAMFIEIVGKNTEES
LQNDLALFNALQEGRQAEMEKKSAEFQEETRKAEETNRIMGCIGKVLGALLTIVSVV
AAVFTGGASLALAAVGLAVMVADEIVKAATGVSFIQQALNPIMEHVLKPLMELIGK
AITKALEGLGVDKKTAEMAGSIVGAIVAAIAMVAVIVVVAVVGKGAAAKLGNALS
KMMGETIKKLVPNVLKQLAQNGSKLFTQGMQRITSGLGNVGSKMGLQTNALSKEL
VGNTLNKVALGMEVTNTAAQSAGGVAEGVFIKNASEALADFMLARFAMDQIQQWL
KQSVEIFGENQKVTAELQKAMSSAVQQNADASRFILRQSRA
S1 nucleic acid sequence
SEQ ID NO: 25
atgcttaatattcaaaattattccgcttctcctcatccggggatcgttgccgaacggccgcagac
tccctcggcgagcgagcacgtcgagactgccgtggtaccgtctaccacagaacatcgcggtacag
atatcatttcattatcgcaggcggctactaaaatccaccaggcacagcagacgctgcagtcaacg
ccaccgatctctgaagagaataatgacgagcgcacgctggcgcgccagcagttgaccagcagcct
gaatgcgctggcgaagtccggcgtgtcattatccgcagaacaaaatgagaacctgcggagcgcgt
tttctgcgccgacgtcggccttatttagcgcttcgcctatggcgcagccgagaacaaccatttct
gatgctgagatttgggatatggtttcccaaaatatatcggcgataggtgacagctatctgggcgt
ttatgaaaacgttgtcgcagtctataccgatttttatcaggccttcagtgatattctttccaaaa
tgggaggctggttattaccaggtaaggacggtaataccgttaagctagatgttacctcactcaaa
aatgatttaaacagtttagtcaataaatataatcaaataaacagtaataccgttttatttccagc
gcagtcaggcagcggcgttaaagtagccactgaagcggaagcgagacagtggctcagtgaattga
atttaccgaatagctgcctgaaatcttatggatccggttatgtcgtcaccgttgatctgacgcca
ttacaaaaaatggttcaggatattgatggtttaggcgcgccgggaaaagactcaaaactcgaaat
ggataacgccaaatatcaagcctggcagtcgggttttaaagcgcaggaagaaaatatgaaaacca
cattacagacgctgacgcaaaaatatagcaatgccaattcattgtacgacaacctggtaaaagtg
ctgagcagtacgataagtagcagcctggaaaccgccaaaagcttcctgcaaggagtcgacatggt
aaatgacgcaagtagcattagccgtagcggatatacccaaaatccgcgcctcgctgaggcggctt
ttgaaggcgttcgtaagaacacggactttttaaaagcggcggataaagcttttaaagatgtggtg
gcaacgaaagcgggcgaccttaaagccggaacaaagtccggcgagagcgctattaatacggtggg
tctaaagccgcctacggacgccgcccgggaaaaactctccagcgaagggcaattgacattactgc
ttggcaagttaatgaccctactgggcgatgtttcgctgtctcaactggagtctcgtctggcggta
tggcaggcgatgattgagtcacaaaaagagatggggattcaggtatcgaaagaattccagacggc
tctgggagaggctcaggaggcgacggatctctatgaagccagtatcaaaaagacggataccgcca
agagtgtttatgacgctgcgaccaaaaaactgacgcaggcgcaaaataaattgcaatcgctggac
ccggctgaccccggctatgcacaagctgaagccgcggtagaacaggccggaaaagaagcgacaga
ggcgaaagaggccttagataaggccacggatgcgacggttaaagcaggcacagacgccaaagcga
aagccgagaaagcggataacattctgaccaaattccagggaacggctaatgccgcctctcagaat
caggtttcccagggtgagcaggataatctgtcaaatgtcgcccgcctcactatgctcatggccat
gtttattgagattgtgggcaaaaatacggaagaaagcctgcaaaacgatcttgcgcttttcaacg
ccttgcaggaagggcgtcaggcggagatggaaaagaaatcggctgaattccaggaagagacgcgc
aaagccgaggaaacgaaccgcattatgggatgtatcgggaaagtcctcggcgcgctgctaaccat
tgtcagcgttgtggccgctgtttttaccggtggggcgagtctggcgctggctgcggtgggacttg
cggtaatggtggccgatgaaattgtgaaggcggcgacgggagtgtcgtttattcagcaggcgcta
aacccgattatggagcatgtgctgaagccgttaatggagctgattggcaaggcgattaccaaagc
gctggaaggattaggcgtcgataagaaaacggcagagatggccggcagcattgttggtgcgattg
tcgccgctattgccatggtggcggtcattgtggtggtcgcagttgtcgggaaaggcgcggcggcg
aaactgggtaacgcgctgagcaaaatgatgggcgaaacgattaagaagttggtgcctaacgtgct
gaaacagttggcgcaaaacggcagcaaactctttacccaggggatgcaacgtattactagcggtc
tgggtaatgtgggtagcaagatgggcctgcaaacgaatgccttaagtaaagagctggtaggtaat
accctaaataaagtggcgttgggcatggaagtcacgaataccgcagcccagtcagccggtggtgt
tgccgagggcgtatttattaaaaatgccagcgaggcgcttgctgattttatgctcgcccgttttg
ccatggatcagattcagcagtggcttaaacaatccgtagaaatatttggtgaaaaccagaaggta
acggcggaactgcaaaaagccatgtcttctgcggtacagcaaaatgcggatgcttcgcgttttat
tctgcgccagagtcgcgcataa
S1 amino acid sequence
SEQ ID NO: 26
MLNIQNYSASPHPGIVAERPQTPSASEHVETAVVPSTTEHRGTDIISLSQAATKI
HQAQQTLQSTPPISEENNDERTLARQQLTSSLNALAKSGVSLSAEQNENLRSAFSAPT
SALFSASPMAQPRTTISDAEIWDMVSQNISAIGDSYLGVYENVVAVYTDFYQAFSDIL
SKMGGWLLPGKDGNTVKLDVTSLKNDLNSLVNKYNQINSNTVLFPAQSGSGVKVA
TEAEARQWLSELNLPNSCLKSYGSGYVVTVDLTPLQKMVQDIDGLGAPGKDSKLEM
DNAKYQAWQSGFKAQEENMKTTLQTLTQKYSNANSLYDNLVKVLSSTISSSLETAK
SFLQGVDMVNDASSISRSGYTQNPRLAEAAFEGVRKNTDFLKAADKAFKDVVATKA
GDLKAGTKSGESAINTVGLKPPTDAAREKLSSEGQLTLLLGKLMTLLGDVSLSQLES
RLAVWQAMIESQKEMGIQVSKEFQTALGEAQEATDLYEASIKKTDTAKSVYDAATK
KLTQAQNKLQSLDPADPGYAQAEAAVEQAGKEATEAKEALDKATDATVKAGTDA
KAKAEKADNILTKFQGTANAASQNQVSQGEQDNLSNVARLTMLMAMFIEIVGKNTE
ESLQNDLALFNALQEGRQAEMEKKSAEFQEETRKAEETNRIMGCIGKVLGALLTIVS
VVAAVFTGGASLALAAVGLAVMVADEIVKAATGVSFIQQALNPIMEHVLKPLMELI
GKAITKALEGLGVDKKTAEMAGSIVGAIVAAIAMVAVIVVVAVVGKGAAAKLGNA
LSKMMGETIKKLVPNVLKQLAQNGSKLFTQGMQRITSGLGNVGSKMGLQTNALSKE
LVGNTLNKVALGMEVTNTAAQSAGGVAEGVFIKNASEALADFMLARFAMDQIQQW
LKQSVEIFGENQKVTAELQKAMSSAVQQNADASRFILRQSRA
LTA1-GSAAS-S1 Nucleic acid sequence
SEQ ID NO: 27
CATatggacaatggcgatcgtttataccgtgccgactcgcgtcccccagatgagattaaacgtag
cggtgggttaatgccacgtgggcacaatgagtattttgaccgtggaacacagatgaacattaacc
tttacgatcatgcccgtgggacccagaccgggtttgtccgttatgatgacgggtatgttagtacg
agtttgtccttacgctccgcacaccttgcgggacaaagtattttatcaggctacagcacatatta
catttatgtgatcgccactgccccaaacatgttcaatgtgaacgatgtgttgggggtttacagcc
cccatccatatgaacaagaagtctcggcccttggggggatcccatatagccagatttatggttgg
taccgcgtaaattttggtgtgattgatgaacgtttgcatcgtaaccgtgaataccgcgatcgcta
ctaccgtaacttgaacattgcacctgccgaggacggctatcgtttagcgggattcccacccgatc
atcaggcgtggcgtgaggaaccgtggatccatcacgcccctcaggggtgcgggaacagtagtcgc
gggtccgcggcatccatgcttaatattcaaaattattccgcttctcctcatccggggatcgttgc
cgaacggccgcagactccctcggcgagcgagcacgtcgagactgccgtggtaccgtctaccacag
aacatcgcggtacagatatcatttcattatcgcaggcggctactaaaatccaccaggcacagcag
acgctgcagtcaacgccaccgatctctgaagagaataatgacgagcgcacgctggcgcgccagca
gttgaccagcagcctgaatgcgctggcgaagtccggcgtgtcattatccgcagaacaaaatgaga
acctgcggagcgcgttttctgcgccgacgtcggccttatttagcgcttcgcctatggcgcagccg
agaacaaccatttctgatgctgagatttgggatatggtttcccaaaatatatcggcgataggtga
cagctatctgggcgtttatgaaaacgttgtcgcagtctataccgatttttatcaggccttcagtg
atattctttccaaaatgggaggctggttattaccaggtaaggacggtaataccgttaagctagat
gttacctcactcaaaaatgatttaaacagtttagtcaataaatataatcaaataaacagtaatac
cgttttatttccagcgcagtcaggcagcggcgttaaagtagccactgaagcggaagcgagacagt
ggctcagtgaattgaatttaccgaatagctgcctgaaatcttatggatccggttatgtcgtcacc
gttgatctgacgccattacaaaaaatggttcaggatattgatggtttaggcgcgccgggaaaaga
ctcaaaactcgaaatggataacgccaaatatcaagcctggcagtcgggttttaaagcgcaggaag
aaaatatgaaaaccacattacagacgctgacgcaaaaatatagcaatgccaattcattgtacgac
aacctggtaaaagtgctgagcagtacgataagtagcagcctggaaaccgccaaaagcttcctgca
aggagtcgacatggtaaatgacgcaagtagcattagccgtagcggatatacccaaaatccgcgcc
tcgctgaggcggcttttgaaggcgttcgtaagaacacggactttttaaaagcggcggataaagct
tttaaagatgtggtggcaacgaaagcgggcgaccttaaagccggaacaaagtccggcgagagcgc
tattaatacggtgggtctaaagccgcctacggacgccgcccgggaaaaactctccagcgaagggc
aattgacattactgcttggcaagttaatgaccctactgggcgatgtttcgctgtctcaactggag
tctcgtctggcggtatggcaggcgatgattgagtcacaaaaagagatggggattcaggtatcgaa
agaattccagacggctctgggagaggctcaggaggcgacggatctctatgaagccagtatcaaaa
agacggataccgccaagagtgtttatgacgctgcgaccaaaaaactgacgcaggcgcaaaataaa
ttgcaatcgctggacccggctgaccccggctatgcacaagctgaagccgcggtagaacaggccgg
aaaagaagcgacagaggcgaaagaggccttagataaggccacggatgcgacggttaaagcaggca
cagacgccaaagcgaaagccgagaaagcggataacattctgaccaaattccagggaacggctaat
gccgcctctcagaatcaggtttcccagggtgagcaggataatctgtcaaatgtcgcccgcctcac
tatgctcatggccatgtttattgagattgtgggcaaaaatacggaagaaagcctgcaaaacgatc
ttgcgcttttcaacgccttgcaggaagggcgtcaggcggagatggaaaagaaatcggctgaattc
caggaagagacgcgcaaagccgaggaaacgaaccgcattatgggatgtatcgggaaagtcctcgg
cgcgctgctaaccattgtcagcgttgtggccgctgtttttaccggtggggcgagtctggcgctgg
ctgcggtgggacttgcggtaatggtggccgatgaaattgtgaaggcggcgacgggagtgtcgttt
attcagcaggcgctaaacccgattatggagcatgtgctgaagccgttaatggagctgattggcaa
ggcgattaccaaagcgctggaaggattaggcgtcgataagaaaacggcagagatggccggcagca
ttgttggtgcgattgtcgccgctattgccatggtggcggtcattgtggtggtcgcagttgtcggg
aaaggcgcggcggcgaaactgggtaacgcgctgagcaaaatgatgggcgaaacgattaagaagtt
ggtgcctaacgtgctgaaacagttggcgcaaaacggcagcaaactctttacccaggggatgcaac
gtattactagcggtctgggtaatgtgggtagcaagatgggcctgcaaacgaatgccttaagtaaa
gagctggtaggtaataccctaaataaagtggcgttgggcatggaagtcacgaataccgcagccca
gtcagccggtggtgttgccgagggcgtatttattaaaaatgccagcgaggcgcttgctgatttta
tgctcgcccgttttgccatggatcagattcagcagtggcttaaacaatccgtagaaatatttggt
gaaaaccagaaggtaacggcggaactgcaaaaagccatgtcttctgcggtacagcaaaatgcgga
tgcttcgcgttttattctgcgccagagtcgcgcataaCTCGAG
LTA1-GSAAS-S1 Amino acid sequence
SEQ ID NO: 28
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRGSAASMLNIQNYSASPHPGIVAERPQTP
SASEHVETAVVPSTTEHRGTDIISLSQAATKIHQAQQTLQSTPPISEENNDERTLARQQ
LTSSLNALAKSGVSLSAEQNENLRSAFSAPTSALFSASPMAQPRTTISDAEIWDMVSQ
NISAIGDSYLGVYENVVAVYTDFYQAFSDILSKMGGWLLPGKDGNTVKLDVTSLKN
DLNSLVNKYNQINSNTVLFPAQSGSGVKVATEAEARQWLSELNLPNSCLKSYGSGY
VVTVDLTPLQKMVQDIDGLGAPGKDSKLEMDNAKYQAWQSGFKAQEENMKTTLQ
TLTQKYSNANSLYDNLVKVLSSTISSSLETAKSFLQGVDMVNDASSISRSGYTQNPRL
AEAAFEGVRKNTDFLKAADKAFKDVVATKAGDLKAGTKSGESAINTVGLKPPTDA
AREKLSSEGQLTLLLGKLMTLLGDVSLSQLESRLAVWQAMIESQKEMGIQVSKEFQT
ALGEAQEATDLYEASIKKTDTAKSVYDAATKKLTQAQNKLQSLDPADPGYAQAEA
AVEQAGKEATEAKEALDKATDATVKAGTDAKAKAEKADNILTKFQGTANAASQNQ
VSQGEQDNLSNVARLTMLMAMFIEIVGKNTEESLQNDLALFNALQEGRQAEMEKKS
AEFQEETRKAEETNRIMGCIGKVLGALLTIVSVVAAVFTGGASLALAAVGLAVMVA
DEIVKAATGVSFIQQALNPIMEHVLKPLMELIGKAITKALEGLGVDKKTAEMAGSIV
GAIVAAIAMVAVIVVVAVVGKGAAAKLGNALSKMMGETIKKLVPNVLKQLAQNGS
KLFTQGMQRITSGLGNVGSKMGLQTNALSKELVGNTLNKVALGMEVTNTAAQSAG
GVAEGVFIKNASEALADFMLARFAMDQIQQWLKQSVEIFGENQKVTAELQKAMSSA
VQQNADASRFILRQSRA*
His-SscA chaperone for S2 nucleic acid sequence
SEQ ID NO: 29
ATGGGCAGCAGCCATCACCATCATCACCACAGCCAGGATCCGatgaaaaaagac
ccgaccctacaacaggcacatgacacgatgcggtttttccggcgtggcggctcgctgcgtatgtt
gttggatgacgatgttacacagccgcttaatactctgtatcgctatgccacgcagcttatggagg
taaaagaattcgccggcgcagcgcgactttttcaattgctgacgatatatgatgcctggtcattt
gactactggtttcggttaggggaatgctgccaggctcaaaaacattggggggaagcgatatacgc
ttatggacgcgcggcacaaattaagattgatgcgccgcaggcgccatgggccgcagcggaatgct
atctcgcgtgtgataacgtctgttatgcaatcaaagcgttaaaggccgtggtgcgtatttgcggc
gaggtcagtgaacatcaaattctccgacagcgtgcagaaaagatgttacagcaactttctgacag
gagctaaAAGCTT
His-SscA chaperone for S2 Amino acid sequence
SEQ ID NO: 30
MGSSHHHHHHSQDPMKKDPTLQQAHDTMRFFRRGGSLRMLLDDDVTQPLN
TLYRYATQLMEVKEFAGAARLFQLLTIYDAWSFDYWFRLGECCQAQKHWGEAIYA
YGRAAQIKIDAPQAPWAAAECYLACDNVCYAIKALKAVVRICGEVSEHQILRQRAE
KMLQQLSDRS*
SseB nucleic acid sequence
SEQ ID NO: 31
Atgtcttcaggaaacatcttatggggaagtcaaaaccctattgtgtttaaaaatagcttcggcgt
cagcaacgctgataccgggagccaggatgacttatcccagcaaaatccgtttgccgaagggtatg
gtgttttgcttattctccttatggttattcaggctatcgcaaataataaatttattgaagtccag
aagaacgctgaacgtgccagaaatacccaggaaaagtcaaatgagatggatgaggtgattgctaa
agcagccaaaggggatgctaaaaccaaagaggaggtgcctgaggatgtaattaaatacatgcgtg
ataatggtattctcatcgatggtatgaccattgatgattatatggctaaatatggcgatcatggg
aagctggataaaggtggcctacaggcgatcaaagcggctttggataatgacgccaaccggaatac
cgatcttatgagtcaggggcagataacaattcaaaaaatgtctcaggagcttaacgctgtcctta
cccaactgacagggcttatcagtaagtggggggaaatttccagtatgatagcgcagaaaacgtac
tca
SseB amino acid sequence
SEQ ID NO: 32
MSSGNILWGSQNPIVFKNSFGVSNADTGSQDDLSQQNPFAEGYGVLLILLMVI
QAIANNKFIEVQKNAERARNTQEKSNEMDEVIAKAAKGDAKTKEEVPEDVIKYMRD
NGILIDGMTIDDYMAKYGDHGKLDKGGLQAIKAALDNDANRNTDLMSQGQITIQK
MSQELNAVLTQLTGLISKWGEISSMIAQKTYS
SseC nucleic acid sequence
SEQ ID NO: 33
atgaatcgaattcacagtaatagcgacagcgccgcaggagtaaccgccttaacacatcatcactt
aagcaatgtcagttgcgtttcctcgggttcgctgggaaagcgccagcatcgtgtgaattctactt
ttggcgatggcaacgccgcgtgtctgctatccgggaaaattagtcttcaggaggcaagcaatgcg
ttgaagcaactgcttgatgccgtacccggaaatcataagcgtccatcattgcctgactttttgca
gaccaatcccgcggttttatcaatgatgatgacgtcattaatactcaacgtctttggtaataacg
ctcaatcgttatgccaacagcttgagcgggcaactgaggtgcaaaatgcattacgtaataagcag
gtaaaggagtatcaggagcagatccagaaagcgatagagcaggaggataaagcgcgtaaagcggg
tatttttggcgctatttttgactggattaccggcatatttgaaaccgtgattggcgccttaaaag
ttgtggaaggttttctgtccggaaatcccgcagaaatggctagcggcgtagcttatatggccgca
ggttgtgcaggaatggttaaagccggagccgaaacggcaatgatgtgcggtgctgaccacgatac
ctgtcaggcaattattgacgtgacaagtaagattcaatttggttgtgaagccgtcgcgctggcac
tggatgttttccagattggccgtgcttttatggcgacgagaggtttatctggcgcagctgcaaaa
gtgcttgactccggttttggcgaggaagtggttgagcgtatggtaggtgcaggggaagcagaaat
agaggagttggctgaaaagtttggcgaagaagtgagcgaaagtttttccaaacaatttgagccgc
ttgaacgtgaaatggctatggcgaatgagatggcagaggaggctgccgagttttctcgtaacgta
gaaaataatatgacgcgaagcgcgggaaaaagctttacgaaagagggggtgaaagcaatggcaaa
agaagcggcaaaagaagccctggaaaaatgtgtgcaagaaggtggaaagttcctgttaaaaaaat
tccgtaataaagttctcttcaatatgttcaaaaaaatcctgtatgccttactgagggattgttca
tttaaaggcttacaggctatcagatgtgcaaccgagggcgccagtcagatgaatactggcatggt
taacacagaaaaagcgaagatcgaaaagaaaatagagcaattaataactcagcaacggtttctgg
atttcataatgcaacaaacagaaaaccagaaaaagatagaacaaaaacgcttagaggagctttat
aaggggagcggtgccgcgcttagagatgtattagataccattgatcactatagtagcgttcaggc
gagaatagctggctatcgcgcttaa
SseC amino acid sequence
SEQ ID NO: 34
MNRIHSNSDSAAGVTALTHHHLSNVSCVSSGSLGKRQHRVNSTFGDGNAAC
LLSGKISLQEASNALKQLLDAVPGNHKRPSLPDFLQTNPAVLSMMMTSLILNVFGNN
AQSLCQQLERATEVONALRNKQVKEYQEQIQKAIEQEDKARKAGIFGAIFDWITGIFE
TVIGALKVVEGFLSGNPAEMASGVAYMAAGCAGMVKAGAETAMMCGADHDTCQ
AIIDVTSKIQFGCEAVALALDVFQIGRAFMATRGLSGAAAKVLDSGFGEEVVERMVG
AGEAEIEELAEKFGEEVSESFSKQFEPLEREMAMANEMAEEAAEFSRNVENNMTRSA
GKSFTKEGVKAMAKEAAKEALEKCVQEGGKFLLKKFRNKVLFNMFKKILYALLRD
CSFKGLQAIRCATEGASQMNTGMVNTEKAKIEKKIEQLITQQRFLDFIMQQTENQKKI
EQKRLEELYKGSGAALRDVLDTIDHYSSVQARIAGYRA
S2 nucleic acid sequence
SEQ ID NO: 35
atgtcttcaggaaacatcttatggggaagtcaaaaccctattgtgtttaaaaatagcttcggcgt
cagcaacgctgataccgggagccaggatgacttatcccagcaaaatccgtttgccgaagggtatg
gtgttttgcttattctccttatggttattcaggctatcgcaaataataaatttattgaagtccag
aagaacgctgaacgtgccagaaatacccaggaaaagtcaaatgagatggatgaggtgattgctaa
agcagccaaaggggatgctaaaaccaaagaggaggtgcctgaggatgtaattaaatacatgcgtg
ataatggtattctcatcgatggtatgaccattgatgattatatggctaaatatggcgatcatggg
aagctggataaaggtggcctacaggcgatcaaagcggctttggataatgacgccaaccggaatac
cgatcttatgagtcaggggcagataacaattcaaaaaatgtctcaggagcttaacgctgtcctta
cccaactgacagggcttatcagtaagtggggggaaatttccagtatgatagcgcagaaaacgtac
tcaGAGCTCatgaatcgaattcacagtaatagcgacagcgccgcaggagtaaccgccttaacaca
tcatcacttaagcaatgtcagttgcgtttcctcgggttcgctgggaaagcgccagcatcgtgtga
attctacttttggcgatggcaacgccgcgtgtctgctatccgggaaaattagtcttcaggaggca
agcaatgcgttgaagcaactgcttgatgccgtacccggaaatcataagcgtccatcattgcctga
ctttttgcagaccaatcccgcggttttatcaatgatgatgacgtcattaatactcaacgtctttg
gtaataacgctcaatcgttatgccaacagcttgagcgggcaactgaggtgcaaaatgcattacgt
aataagcaggtaaaggagtatcaggagcagatccagaaagcgatagagcaggaggataaagcgcg
taaagcgggtatttttggcgctatttttgactggattaccggcatatttgaaaccgtgattggcg
ccttaaaagttgtggaaggttttctgtccggaaatcccgcagaaatggctagcggcgtagcttat
atggccgcaggttgtgcaggaatggttaaagccggagccgaaacggcaatgatgtgcggtgctga
ccacgatacctgtcaggcaattattgacgtgacaagtaagattcaatttggttgtgaagccgtcg
cgctggcactggatgttttccagattggccgtgcttttatggcgacgagaggtttatctggcgca
gctgcaaaagtgcttgactccggttttggcgaggaagtggttgagcgtatggtaggtgcagggga
agcagaaatagaggagttggctgaaaagtttggcgaagaagtgagcgaaagtttttccaaacaat
ttgagccgcttgaacgtgaaatggctatggcgaatgagatggcagaggaggctgccgagttttct
cgtaacgtagaaaataatatgacgcgaagcgcgggaaaaagctttacgaaagagggggtgaaagc
aatggcaaaagaagcggcaaaagaagccctggaaaaatgtgtgcaagaaggtggaaagttcctgt
taaaaaaattccgtaataaagttctcttcaatatgttcaaaaaaatcctgtatgccttactgagg
gattgttcatttaaaggcttacaggctatcagatgtgcaaccgagggcgccagtcagatgaatac
tggcatggttaacacagaaaaagcgaagatcgaaaagaaaatagagcaattaataactcagcaac
ggtttctggatttcataatgcaacaaacagaaaaccagaaaaagatagaacaaaaacgcttagag
gagctttataaggggagcggtgccgcgcttagagatgtattagataccattgatcactatagtag
cgttcaggcgagaatagctggctatcgcgcttaa
S2 amino acid sequence
SEQ ID NO: 36
MSSGNILWGSQNPIVFKNSFGVSNADTGSQDDLSQQNPFAEGYGVLLILLMVI
QAIANNKFIEVQKNAERARNTQEKSNEMDEVIAKAAKGDAKTKEEVPEDVIKYMRD
NGILIDGMTIDDYMAKYGDHGKLDKGGLQAIKAALDNDANRNTDLMSQGQITIQK
MSQELNAVLTQLTGLISKWGEISSMIAQKTYSELMNRIHSNSDSAAGVTALTHHHLS
NVSCVSSGSLGKRQHRVNSTFGDGNAACLLSGKISLQEASNALKQLLDAVPGNHKR
PSLPDFLQTNPAVLSMMMTSLILNVFGNNAQSLCQQLERATEVQNALRNKQVKEYQ
EQIQKAIEQEDKARKAGIFGAIFDWITGIFETVIGALKVVEGFLSGNPAEMASGVAYM
AAGCAGMVKAGAETAMMCGADHDTCQAIIDVTSKIQFGCEAVALALDVFQIGRAF
MATRGLSGAAAKVLDSGFGEEVVERMVGAGEAEIEELAEKFGEEVSESFSKQFEPLE
REMAMANEMAEEAAEFSRNVENNMTRSAGKSFTKEGVKAMAKEAAKEALEKCVQ
EGGKFLLKKFRNKVLFNMFKKILYALLRDCSFKGLQAIRCATEGASQMNTGMVNTE
KAKIEKKIEQLITQQRFLDFIMQQTENQKKIEQKRLEELYKGSGAALRDVLDTIDHYS
SVQARIAGYRA
LTA1-GSAAS-S2 nucleic acid sequence
SEQ ID NO: 37
CATatggacaatggcgatcgtttataccgtgccgactcgcgtcccccagatgagattaaacgtag
cggtgggttaatgccacgtgggcacaatgagtattttgaccgtggaacacagatgaacattaacc
tttacgatcatgcccgtgggacccagaccgggtttgtccgttatgatgacgggtatgttagtacg
agtttgtccttacgctccgcacaccttgcgggacaaagtattttatcaggctacagcacatatta
catttatgtgatcgccactgccccaaacatgttcaatgtgaacgatgtgttgggggtttacagcc
cccatccatatgaacaagaagtctcggcccttggggggatcccatatagccagatttatggttgg
taccgcgtaaattttggtgtgattgatgaacgtttgcatcgtaaccgtgaataccgcgatcgcta
ctaccgtaacttgaacattgcacctgccgaggacggctatcgtttagcgggattcccacccgatc
atcaggcgtggcgtgaggaaccgtggatccatcacgcccctcaggggtgcgggaacagtagtcgc
gggtccgcggcatccatgtcttcaggaaacatcttatggggaagtcaaaaccctattgtgtttaa
aaatagcttcggcgtcagcaacgctgataccgggagccaggatgacttatcccagcaaaatccgt
ttgccgaagggtatggtgttttgcttattctccttatggttattcaggctatcgcaaataataaa
tttattgaagtccagaagaacgctgaacgtgccagaaatacccaggaaaagtcaaatgagatgga
tgaggtgattgctaaagcagccaaaggggatgctaaaaccaaagaggaggtgcctgaggatgtaa
ttaaatacatgcgtgataatggtattctcatcgatggtatgaccattgatgattatatggctaaa
tatggcgatcatgggaagctggataaaggtggcctacaggcgatcaaagcggctttggataatga
cgccaaccggaataccgatcttatgagtcaggggcagataacaattcaaaaaatgtctcaggagc
ttaacgctgtccttacccaactgacagggcttatcagtaagtggggggaaatttccagtatgata
gcgcagaaaacgtactcaGAGCTCatgaatcgaattcacagtaatagcgacagcgccgcaggagt
aaccgccttaacacatcatcacttaagcaatgtcagttgcgtttcctcgggttcgctgggaaagc
gccagcatcgtgtgaattctacttttggcgatggcaacgccgcgtgtctgctatccgggaaaatt
agtcttcaggaggcaagcaatgcgttgaagcaactgcttgatgccgtacccggaaatcataagcg
tccatcattgcctgactttttgcagaccaatcccgcggttttatcaatgatgatgacgtcattaa
tactcaacgtctttggtaataacgctcaatcgttatgccaacagcttgagcgggcaactgaggtg
caaaatgcattacgtaataagcaggtaaaggagtatcaggagcagatccagaaagcgatagagca
ggaggataaagcgcgtaaagcgggtatttttggcgctatttttgactggattaccggcatatttg
aaaccgtgattggcgccttaaaagttgtggaaggttttctgtccggaaatcccgcagaaatggct
agcggcgtagcttatatggccgcaggttgtgcaggaatggttaaagccggagccgaaacggcaat
gatgtgcggtgctgaccacgatacctgtcaggcaattattgacgtgacaagtaagattcaatttg
gttgtgaagccgtcgcgctggcactggatgttttccagattggccgtgcttttatggcgacgaga
ggtttatctggcgcagctgcaaaagtgcttgactccggttttggcgaggaagtggttgagcgtat
ggtaggtgcaggggaagcagaaatagaggagttggctgaaaagtttggcgaagaagtgagcgaaa
gtttttccaaacaatttgagccgcttgaacgtgaaatggctatggcgaatgagatggcagaggag
gctgccgagttttctcgtaacgtagaaaataatatgacgcgaagcgcgggaaaaagctttacgaa
agagggggtgaaagcaatggcaaaagaagcggcaaaagaagccctggaaaaatgtgtgcaagaag
gtggaaagttcctgttaaaaaaattccgtaataaagttctcttcaatatgttcaaaaaaatcctg
tatgccttactgagggattgttcatttaaaggcttacaggctatcagatgtgcaaccgagggcgc
cagtcagatgaatactggcatggttaacacagaaaaagcgaagatcgaaaagaaaatagagcaat
taataactcagcaacggtttctggatttcataatgcaacaaacagaaaaccagaaaaagatagaa
caaaaacgcttagaggagctttataaggggagcggtgccgcgcttagagatgtattagataccat
tgatcactatagtagcgttcaggcgagaatagctggctatcgcgcttaaCTCGAG
LTA1-GSAAS-S2 Amino acid sequence
SEQ ID NO: 38
MDNGDRLYRADSRPPDEIKRSGGLMPRGHNEYFDRGTQMNINLYDHARGTQ
TGFVRYDDGYVSTSLSLRSAHLAGQSILSGYSTYYIYVIATAPNMFNVNDVLGVYSP
HPYEQEVSALGGIPYSQIYGWYRVNFGVIDERLHRNREYRDRYYRNLNIAPAEDGYR
LAGFPPDHQAWREEPWIHHAPQGCGNSSRGSAASMSSGNILWGSQNPIVFKNSFGVS
NADTGSQDDLSQQNPFAEGYGVLLILLMVIQAIANNKFIEVQKNAERARNTQEKSNE
MDEVIAKAAKGDAKTKEEVPEDVIKYMRDNGILIDGMTIDDYMAKYGDHGKLDKG
GLQAIKAALDNDANRNTDLMSQGQITIQKMSQELNAVLTQLTGLISKWGEISSMIAQ
KTYSELMNRIHSNSDSAAGVTALTHHHLSNVSCVSSGSLGKRQHRVNSTFGDGNAA
CLLSGKISLQEASNALKQLLDAVPGNHKRPSLPDFLQTNPAVLSMMMTSLILNVFGN
NAQSLCQQLERATEVQNALRNKQVKEYQEQIQKAIEQEDKARKAGIFGAIFDWITGI
FETVIGALKVVEGFLSGNPAEMASGVAYMAAGCAGMVKAGAETAMMCGADHDTC
QAIIDVTSKIQFGCEAVALALDVFQIGRAFMATRGLSGAAAKVLDSGFGEEVVERMV
GAGEAEIEELAEKFGEEVSESFSKQFEPLEREMAMANEMAEEAAEFSRNVENNMTRS
AGKSFTKEGVKAMAKEAAKEALEKCVQEGGKFLLKKFRNKVLFNMFKKILYALLR
DCSFKGLQAIRCATEGASQMNTGMVNTEKAKIEKKIEQLITQQRFLDFIMQQTENQK
KIEQKRLEELYKGSGAALRDVLDTIDHYSSVQARIAGYRA*
LTA1-SseB Nucleic acid sequence
SEQ ID NO: 39
ATGGGCAGCAGCCATCATCATCATCATCACAGCAGCGGCCTGGTGCCGCG
CGGCAGCCATatggacaatggcgatcgtttataccgtgccgactcgcgtcccccagatgagatta
aacgtagcggtgggttaatgccacgtgggcacaatgagtattttgaccgtggaacacagatgaac
attaacctttacgatcatgcccgtgggacccagaccgggtttgtccgttatgatgacgggtatgt
tagtacgagtttgtccttacgctccgcacaccttgcgggacaaagtattttatcaggctacagca
catattacatttatgtgatcgccactgccccaaacatgttcaatgtgaacgatgtgttgggggtt
tacagcccccatccatatgaacaagaagtctcggcccttggggggatcccatatagccagattta
tggttggtaccgcgtaaattttggtgtgattgatgaacgtttgcatcgtaaccgtgaataccgcg
atcgctactaccgtaacttgaacattgcacctgccgaggacggctatcgtttagcgggattccca
cccgatcatcaggcgtggcgtgaggaaccgtggatccatcacgcccctcaggggtgcgggaacag
tagtcgcgggtccgcggcatccatgtcttcaggaaacatcttatggggaagtcaaaaccctattg
tgtttaaaaatagcttcggcgtcagcaacgctgataccgggagccaggatgacttatcccagcaa
aatccgtttgccgaagggtatggtgttttgcttattctccttatggttattcaggctatcgcaaa
taataaatttattgaagtccagaagaacgctgaacgtgccagaaatacccaggaaaagtcaaatg
agatggatgaggtgattgctaaagcagccaaaggggatgctaaaaccaaagaggaggtgcctgag
gatgtaattaaatacatgcgtgataatggtattctcatcgatggtatgaccattgatgattatat
ggctaaatatggcgatcatgggaagctggataaaggtggcctacaggcgatcaaagcggctttgg
ataatgacgccaaccggaataccgatcttatgagtcaggggcagataacaattcaaaaaatgtct
caggagcttaacgctgtccttacccaactgacagggcttatcagtaagtggggggaaatttccag
tatgatagcgcagaaaacgtactcataaGGATCC
LTA1-SseB Amino acid sequence
SEQ ID NO: 40
MGSSHHHHHHSSGLVPRGSHMDNGDRLYRADSRPPDEIKRSGGLMPRGHNE
YFDRGTQMNINLYDHARGTQTGFVRYDDGYVSTSLSLRSAHLAGQSILSGYSTYYIY
VIATAPNMFNVNDVLGVYSPHPYEQEVSALGGIPYSQIYGWYRVNFGVIDERLHRNR
EYRDRYYRNLNIAPAEDGYRLAGFPPDHQAWREEPWIHHAPQGCGNSSRGSAASMS
SGNILWGSQNPIVFKNSFGVSNADTGSQDDLSQQNPFAEGYGVLLILLMVIQAIANN
KFIEVQKNAERARNTQEKSNEMDEVIAKAAKGDAKTKEEVPEDVIKYMRDNGILID
GMTIDDYMAKYGDHGKLDKGGLQAIKAALDNDANRNTDLMSQGQITIQKMSQELN
AVLTQLTGLISKWGEISSMIAQKTYS*
DBF Amino acid sequence
SEQ ID NO: 41
GSAASMNITTLTNSISTSSFSPNNTNGSSTETVNSDIKTTTSSHPSSLTMLNDTL
HNIRTTNQALKKELSQKTLRNEYPINKDARELLHSAPKEAELDGDQMISHRELWAKI
ANSINDINEQYLKVYEHAVSSYTQMYQDFSAVLSSLAGWISPGGNDGNSVKLQVNS
LKKALEELKEKYKDKPLYPANNTVSQEQANKWLTELGGTIGKVSQKNGGYVVSIN
MTPIDNMLKSLDNLGGNGEVVLDNAKYQAWNGFSAEDETMKNNLQTLVQKYSNA
NSIFDNLVKVLSSTISSCTDTDKLFLHFLEMHNVSTTTTGFPLAKILTSTELGDNTIQA
ANDAANKLESLTIADLTANQNINTTNAHSTSNILIPELKAPKSLNASSQLTLLIGNLIQI
LGEKSLTALTNKITAWKSQQQARQQKNLEFSDKINTLLSETEGLTRDYEKQINKLKN
ADSKIKDLENKINQIQTRLSNLDPESPEKKKLSREEIQLTIKKDAAVKDRTLIEQKTLSI
HSKLTDKSMQLEKEIDSFSAFSNTASAEQLSTQQKSLTGLASVTQLMATFIQLVGKN
NEESLKNDLALFQSLQESRKTEMERKSDEYAAEVRKAEELNRVMGCVGKILGALLTI
VSVVAAAFSGGASLALAAVGLALMVTDAIVQAATGNSFMEQALNPIMKAVIEPLIKL
LSDAFTKMLEGLGVDSKKAKMIGSILGAIAGALVLVAAVVLVATVGKQAAAKLAE
NIGKIIGKTLTDLIPKFLKNFSSQLDDLITNAVARLNKFLGAAGDEVISKQIISTHLNQA
VLLGESVNSATQAGGSVASAVFQNSASTNLADLTLSKYQVEQLSKYISEAIEKFGQL
QEVIADLLASMSNSQANRTDVAKAILQQTTA

1. A fusion polypeptide comprising a fusion of a needle tip protein or an antigenic fragment thereof and a translocator protein or an antigenic fragment thereof from a Type III secretion system (T3SS) of Salmonella enterica. or Shigella spp.

2. The polypeptide of claim 1, wherein the fusion polypeptide is arranged so that the needle tip protein is 5′ of the translocator protein.

3. The polypeptide of claim 1, wherein the needle tip protein comprises IpaD, SipD, or SseB.

4. The polypeptide of claim 1, wherein the translocator protein comprises IpaB, SipB, or SseC.

5. The polypeptide of claim 1, wherein the fusion comprises the Shigella spp. needle-tip protein (IpaD) and translocator protein (IpaB), or antigenic fragments thereof.

6. The polypeptide of claim 5, wherein the fusion comprises the sequence as set forth in SEQ ID NO: 41.

7. The polypeptide of claim 1, wherein the fusion comprises the Salmonella spp. needle-tip protein (SipD) and translocator protein (SipB), or antigenic fragments thereof.

8. The polypeptide of claim 5, wherein the fusion comprises the sequence as set forth in SEQ ID NO: 26.

9. The polypeptide of claim 1, wherein the fusion comprises the Salmonella spp. needle-tip protein (SseB) and translocator protein (SseC), or antigenic fragments thereof.

10. The polypeptide of claim 5, wherein the fusion comprises the sequence as set forth in SEQ ID NO: 36.

11. The polypeptide of claim 1, wherein the fusion further comprises double mutant labile toxin (dmLT) or an antigenic fragment thereof from Enterotoxigenic Escherichia coli or cholera toxin or an antigenic fragment thereof.

12. The polypeptide of claim 1, wherein the dmLT comprises the active moiety LTA1.

13. The polypeptide of claim 1, wherein the dmLT retains its ADP ribosylation activity.

14. The polypeptide of claim 1, wherein the dmLT is 5′ of the needle tip protein and translocator protein fusion.

15. The polypeptide of claim 1, wherein polypeptide comprises the amino acid sequence as set forth in SEQ ID NO: 16, SEQ ID NO: 28, or SEQ ID NO: 38.

16. A vaccine comprising the fusion claim 1.

17. (canceled)

18. The vaccine of claim 16, further comprising pertussis toxoid (PTd) or Bacterial Enzymatic Combinatorial Chemistry candidate 438 (BECC438).

19. (canceled)

20. (canceled)

21. (canceled)

22. A method of treating, inhibiting, or preventing an infection of a Gram negative bacteria in a subject comprising administering to the subject the fusion polypeptide of claim 1.

23. The method of claim 22, wherein the method further inhibits or prevents colony formation of the bacteria and/or transmission of the bacteria to another subject.

24. A method of eliciting an immune response in a subject to a Gram negative bacteria comprising administering to the subject the fusion polypeptide of claim 1.

25. (canceled)

26. (canceled)

27. (canceled)
